(19) United States
(12) Patent Application Publication
(10) Pub. No.: US 2025/0205256 A1
(43) Pub. Date: Jun. 26, 2025

(54) 5-MEO-DMT FOR USE IN THE TREATMENT OF NEGATIVE THINKING

(71) Applicant: GH RESEARCH IRELAND LIMITED, Dublin 2 (IE)

(72) Inventors: Theis TERWEY, Berlin (DE); Conor BURKE, Laytown (IE); Naoise GAFFNEY, Dublin (IE)

(73) Assignee: GH RESEARCH IRELAND LIMITED, Dublin 2 (IE)

(21) Appl. No.: 18/850,383

(22) PCT Filed: Mar. 27, 2023
(86) PCT No.: PCT/EP2023/057867

§ 371 Date: Sep. 24, 2024

(30) Foreign Application Priority Data
Mar. 27, 2022 (EP) 22000083.0
Mar. 27, 2022 (EP) 22000086.3
Jan. 30, 2023 (EP) 23153939.6
Jan. 30, 2023 (EP) 23153995.8

(51) Int. Cl. A61K 31/675; A61P 25/24

(52) U.S. Cl. CPC A61K 31/675; A61P 25/24

(57) ABSTRACT

5-Methoxy-N,N-dimethyltryptamine (5-MeO-DMT) or a pharmaceutically acceptable salt thereof is used in treating a patient who is suffering from negative thinking.

### TECHNICAL FIELD

The present invention is directed to improved methods for the treatment of negative thinking, in particular negative thinking in a patient suffering from a mental or nervous system disorder, such as disorders characterized by depressive episodes, for example, Major Depressive Disorder (MDD), Bipolar Disorder (BD), such as Bipolar I Disorder and Bipolar II Disorder, Postpartum Depression (PPD), Seasonal Affective Disorder and Persistent Depressive Disorder; Anxiety Disorder, for example Generalised Anxiety Disorder (GAD) and Social Anxiety Disorder (SAD); Obsessive Compulsive and Related Disorders, for example, Obsessive Compulsive Disorder (OCD) and Body Dysmorphic Disorder (BDD); Post-Traumatic Stress Disorder (PTSD); Pain Disorders, for example, Chronic Pain; Mental and Behavioural Disorders due to Psychoactive Substance Use, for example Substance Use Disorder (SUD); Psychotic Disorder, for example Schizophrenia; Dementia, for example Alzheimer's Dementia (AD); Eating Disorders; Attention Deficit Hyperactivity Disorder (ADHD); Personality Disorders, for example Schizotypal Personality Disorder and Borderline Personality Disorder (BPD).

The negative thinking can also occur in a patient suffering from sleep disturbance, for instance, insomnia.

The negative thinking can also occur in a patient suffering from a medical health condition leading to an associated mental or nervous system condition including Traumatic Brain Injury (TBI).

The treatment comprises administering to a patient in need thereof a therapeutically effective amount of 5-methoxy-N,N-dimethyltryptamine (5-MeO-DMT) or of a pharmaceutically acceptable salt thereof.

### BACKGROUND OF THE INVENTION

Symptoms such as pessimism, feelings of worthlessness, feelings of helplessness and hopelessness, and feelings of pathological, excessive or inappropriate guilt are clustered together here as negative thinking.

Feelings of helplessness and hopelessness (also simply referred to as helplessness and hopelessness) characterize the subjective sense of pessimism or gloom regarding the future, inability to cope, or sense of loss of control.

Feelings of worthlessness (also simply referred to as worthlessness) characterize the subjective sense or thoughts of decreased self-value or self-worth.

Feelings of guilt (also simply referred to as guilt) characterise the subjective sense of self blame, failure, or remorse for real or imagined past errors.

Negative thinking can not only have a severe impact on the quality of life but can also lead to various secondary health problems.

Thus, there is a need for improved methods for negative thinking, in particular negative thinking associated with a mental disorder or a nervous system disorder.

### SUMMARY OF THE INVENTION

An aim of the invention is in particular the provision of improved therapies which are more effective (i.e., a) a larger percentage of patients experiencing a clinical response, b) a larger average clinical response, c) an earlier onset of the clinical response, and/or d) a more durable clinical response) than previously described therapies.

A further aim of the current invention is to provide a compound for improved psychoactive therapies and dosing regimens for said therapies which have a better safety profile and/or are better tolerated than previously described therapies. Another aim of the current invention is to provide a compound for improved psychoactive therapies and dosing regimens for said therapies which are more convenient than previously described therapies. Another aim of the current invention is to provide a compound for improved psychoactive therapies and dosing regimens for said therapies which are associated with higher rates of patient compliance (including higher rates of treatment initiation) than previously described therapies. A still further aim of the current invention is to identify specific disease aspects and specific subgroups of disease aspects which benefit from such improved psychoactive therapies.

The present invention provides 5-Methoxy-N,N-dimethyltryptamine (5-MeO-DMT) or a pharmaceutically acceptable salt thereof for use in treating a patient suffering from negative thinking, in particular symptoms such as feelings of worthlessness, helplessness and hopelessness, and/or guilt.

The present invention provides improved methods for the treatment of negative thinking, in particular negative thinking in a patient suffering from a mental or nervous system disorder, such as disorders characterized by depressive episodes, for example, Major Depressive Disorder (MDD), Bipolar Disorder (BD), such as Bipolar I Disorder and Bipolar II Disorder, Postpartum Depression (PPD), Seasonal Affective Disorder and Persistent Depressive Disorder; Anxiety Disorder, for example Generalised Anxiety Disorder (GAD) and Social Anxiety Disorder (SAD); Obsessive Compulsive and Related Disorders, for example, Obsessive Compulsive Disorder (OCD) and Body Dysmorphic Disorder (BDD); Post-Traumatic Stress Disorder (PTSD); Pain Disorders, for example, Chronic Pain; Mental and Behavioural Disorders due to Psychoactive Substance Use, for example Substance Use Disorder (SUD); Psychotic Disorder, for example Schizophrenia; Dementia, for example Alzheimer's Dementia (AD); Eating Disorders; Attention Deficit Hyperactivity Disorder (ADHD); Personality Disorders, for example Schizotypal Personality Disorder and Borderline Personality Disorder (BPD).

The invention also provides improved methods for the treatment of negative thinking in a patient suffering from sleep disturbance, for instance, insomnia.

The invention also provides improved methods for the treatment of negative thinking in a patient suffering from a medical health condition leading to an associated mental or nervous system condition including Traumatic Brain Injury (TBI).

The present invention also provides dose ranges and dosing regimen useful for the treatment of negative thinking, in particular feelings of worthlessness, helplessness and hopelessness, and/or guilt.

### DETAILED DESCRIPTION OF THE INVENTION

### Definitions

As used in the context of the present invention, unless otherwise noted, the term “5-MeO-DMT” refers to the free base 5-MeO-DMT. It is contemplated that pharmaceutically acceptable salts of 5-MeO-DMT may also be used. Such salts are in particular acid addition salts, wherein the acid may be selected from, for instance, acetic acid, benzoic acid, citric acid, fumaric acid, hydrobromic acid, hydrochloric acid, hydrofluoric acid, hydroiodic acid, oxalic acid, succinic acid and triflic acid. A preferred example is the hydrobromide salt. The appropriate weight amount of a salt to be administered can be calculated from the weight amount of the free base, assuming that equimolar amounts are used.

As used in the context of the present invention, a “patient” to be treated is a human subject who is suffering from negative thinking by a licensed professional in accordance with accepted medical practice or who is diagnosed by a licensed professional in accordance with accepted medical practice with a mental disorder or a nervous system disorder associated with negative thinking. In the latter case, assessing negative thinking may or may not be part of the diagnosis.

Diagnosis of a mental disorder or a nervous system disorder can, for instance, be in accordance with the Diagnostic and Statistical Manual of Mental Disorders—Fifth Edition (DSM-5) published by the American Psychiatric Association. In some instances, as is apparent from the discussion of specific conditions below, the criteria may be modified or supplemented to better define patients or patient groups particularly benefiting from a treatment according to the invention. The diagnosis will in any event be by a physician or a psychologist. It is not sufficient that the human subject himself/herself considers that he/she is suffering from the disorder.

As used in the context of the present invention, unless otherwise noted, the terms “treating” and “treatment” shall include the management and care of a patient for the purpose of combating a disease, condition, or disorder and includes the administration of compounds and methods according to the present invention to alleviate the signs and/or symptoms of the disease or eliminate the disease, condition, or disorder.

“Treatment of negative thinking” shall include the management and care of a patient for the purpose of combating negative thinking and includes the administration of compounds and methods according to the present invention to alleviate the signs and/or symptoms of negative thinking or eliminate negative thinking.

Negative thinking may be associated with sleep disturbance, for instance, insomnia, a mental disorder or a nervous system disorder or another medical condition.

The patient may suffer from treatment resistant disease. Treatment resistance means that the patient had no adequate improvement after at least two adequate courses of therapy. The patient in particular had no adequate improvement after at least two adequate courses of therapy, wherein at least one of the two courses was a pharmacotherapy; for instance, the patient had no adequate improvement after at least two adequate courses of pharmacotherapy. The at least two prior courses of treatment were in particular administered in the current episode of the disease, for instance, if the patient suffers from a disorder characterized by depressive episodes, in the current episode of depression.

As used in the context of the present invention, unless otherwise noted, the term “therapeutically effective amount” shall mean the amount of active compound or pharmaceutical ingredient that elicits the biological or clinical response in a human that is being sought by a researcher, medical doctor or other clinician, which includes alleviation of the signs and/or symptoms of the disease, condition or disorder being treated. “Clinical response” includes, but is not limited to, improvements on rating scales. These scales assess (i) negative thinking or aspects of negative thinking and/or (ii) a mental disorder or nervous system disorder or aspects of such a disorder and/or (iii) a medical health condition leading to an associated mental or nervous system condition and/or (iv) sleep.

The severity of a condition as well as changes of the severity can be assessed by the Clinical Global Impression (CGI) rating scales which are measures of symptom severity, treatment response and the efficacy of treatments.

The CGI rating scales were developed to provide a brief, stand-alone assessment of the clinician's view of the patient's global functioning prior to and after a treatment (Busner, J. and Tagrum, S. D., 2007. The Clinical Global Impressions Scale: Applying a Research Tool in Clinical Practice. Psychiatry 2007, 29-37).

The CGI-Severity (CGI-S) is based on one question the clinician has to answer: “Considering your total clinical experience with this particular population, how mentally ill is the patient at this time?” This is rated on the following seven-point scale: 1=normal, not at all ill; 2=borderline mentally ill; 3=mildly ill; 4=moderately ill; 5=markedly ill; 6=severely ill; 7=among the most extremely ill patients.

The CGI-S can be used to assess treatment success by comparing scores before and after treatment.

A clinical response may be reflected by a reduction in the Clinical Global Impression—Severity (CGI-S) score. According to the invention, a reduction in the CGI-S score means that the CGI-S is reduced by at least 1. Preferably, the CGI-S is reduced by at least 2 and/or to a score of 0. It is especially preferred if the CGI-S is reduced by at least 3 and/or to a score of 0.

Alternatively, treatment success can be assessed using the CGI-Improvement (CGI-I), which is similarly simple in its format. After the treatment, the clinician compares the patient's overall clinical condition to the one prior to the treatment (the so-called baseline value). Again, only one query is rated on a seven-point scale: “Compared to the patient's condition at admission to the project [prior to medication initiation], this patient's condition is: 1=very much improved since the initiation of treatment; 2=much improved; 3=minimally improved; 4=no change from baseline (the initiation of treatment); 5=minimally worse; 6=much worse; 7=very much worse since the initiation of treatment.” The Patient Global Impression scale (PGI), also known as Subject Global Impression (SGI), is the counterpart to the Clinical Global Impressions scale (CGI). It consists of one item based on the CGI and adapted to the patient. It can measure disease severity (PGI-S) or disease improvement (PGI-I).

Individual items of scales as described herein as well as sub-combinations of individual items may be used to assess specific disease aspects.

As used in the context of the present invention, unless otherwise noted, the term “administration” (or “application”) shall mean the introduction of an amount, which may be a predetermined amount, of active compound or pharmaceutical ingredient into a patient via any route. Preferably, the active compound is administered by inhalation, nasally, by buccal administration or by sublingual administration.

As used in the context of the present invention, unless otherwise noted, the terms “dose” and “dosage” and “dosage amount” shall mean the amount of active compound or pharmaceutical ingredient which is administered to a patient in an individual administration.

The term “dosage regimen” (or “dosing regimen”) shall mean a defined sequence of one or more individual administrations.

As used herein, “aerosol” means a stable system consisting of a gaseous medium (a pharmaceutically acceptable gas, such as air) and miniscule suspended solid and/or liquid particles. The term “degradation product” refers to a compound resulting from a chemical modification of 5-MeO-DMT as a result of a chemical reaction during aerosol formation. Such reaction includes, without limitation, oxidation. When a percentage of a “degradation product” is described in the context of the present invention, then this refers to the quantity of 5-MeO-DMT degradation products present in a sample divided by the quantity of 5-MeO-DMT plus 5-MeO-DMT degradation products present in the sample multiplied by 100%, i.e., (Sum of quantities of all 5-MeO-DMT degradation products present in the sample)/((Quantity of 5-MeO-DMT present in the sample)+(Sum of quantities of all 5-MeO-DMT degradation products present in the sample))×100%. As used herein, the term “impurity” refers to unwanted compounds contaminating a sample of 5-MeO-DMT (or of a pharmaceutically acceptable salt thereof). Impurities may be contained in the starting material before aerosol formation or may be degradation products.

The term “purity” refers to 100% minus the percent of all 5-MeO-DMT degradation products and all other impurities present, i.e., 100%−(Sum of quantities of all 5-MeO-DMT degradation products present+Sum of quantities of all other impurities present)/(Quantity of 5-MeO-DMT present+Sum of quantities of all 5-MeO-DMT degradation products present+Sum of quantities of all other impurities present)×100%.

The term “mass median aerodynamic diameter” (MMAD), is the diameter at which 50% of the particles present in an aerosol are larger than this calculated diameter, and 50% are smaller. The term “aerosol particle mass density” refers to the mass of aerosol particles per unit volume of aerosol. The term “aerosol particle formation rate” refers to the aerosolized mass of 5-MeO-DMT per unit of aerosolization time.

### Negative Thinking

Symptoms such as pessimism, feelings of worthlessness, feelings of helplessness and hopelessness, and feelings of pathological, excessive or inappropriate guilt are clustered together here as negative thinking.

Feelings of helplessness and hopelessness (also simply referred to as helplessness and hopelessness) characterize the subjective sense of pessimism or gloom regarding the future, inability to cope, or sense of loss of control. There is no helplessness and hopelessness if the patient does not have such feelings. Helplessness and hopelessness are mild in the case of occasional and mild feelings of not being able to cope as usual or pessimism; moderate in the case the patient often feels unable to cope or has significant feelings of helplessness or hopelessness which lift at times; or severe if there are marked and persistent feelings of pessimism, helplessness, or hopelessness.

Feelings of worthlessness (also simply referred to as worthlessness) characterize the subjective sense or thoughts of decreased self-value or self-worth. There is no worthlessness if the patient does not have such feelings. They may be mild, i.e., slight decrease in sense of self-worth; moderate; i.e., some thoughts of worthlessness and decreased self-worth; or severe, i.e., marked, pervasive, or persistent feelings of worthlessness, e.g., feels others better off without them, unable to appreciate positive attributes.

Feelings of guilt (also simply referred to as guilt) characterise the subjective sense of self blame, failure, or remorse for real or imagined past errors. There is no guilt if the patient does not have such feelings. They are mild in the case of slight decrease in self-esteem or increased self-criticism; moderate in the case of significant thoughts of failure, self-criticism, inability to cope, or ruminations regarding past failures and the effect on others; able to recognise as excessive; or severe in the case of marked, pervasive, or persistent guilt, e.g., feelings of deserving punishment; or does not clearly recognise as excessive.

Negative thinking can be associated with a mental disorder or a nervous system disorder or some other medical conditions.

Mental or nervous system disorders which lead to, or are associated with, negative thinking include disorders characterized by depressive episodes, for example, Major Depressive Disorder (MDD), Bipolar Disorder (BD), such as Bipolar I Disorder and Bipolar II Disorder, Postpartum Depression (PPD), Seasonal Affective Disorder and Persistent Depressive Disorder; Anxiety Disorder, for example Generalised Anxiety Disorder (GAD) and Social Anxiety Disorder (SAD); Obsessive Compulsive and Related Disorders, for example, Obsessive Compulsive Disorder (OCD) and Body Dysmorphic Disorder (BDD); Post-Traumatic Stress Disorder (PTSD); Pain Disorders, for example, Chronic Pain; Mental and Behavioural Disorders due to Psychoactive Substance Use, for example Substance Use Disorder (SUD); Psychotic Disorder, for example Schizophrenia; Dementia, for example Alzheimer's Dementia (AD); Eating Disorders; Attention Deficit Hyperactivity Disorder (ADHD); Personality Disorders, for example Schizotypal Personality Disorder and Borderline Personality Disorder (BPD).

The negative thinking can also occur in a patient suffering from sleep disturbance, for instance, insomnia.

The negative thinking can also occur in a patient suffering from medical health conditions leading to an associated mental or nervous system condition including Traumatic Brain Injury (TBI).

Measuring Negative thinking

Negative thinking or individual aspects thereof, such as worthlessness, helplessness and hopelessness, and guilt, can be evaluated by different instruments, such as questionnaires or scales.

Questionnaires assess the mental status of a patient based on observations made by the patient himself/herself, caregivers or the clinician administering the questionnaire.

Questionnaires used to assess whether a patient suffers from a particular mental or nervous system disorder may comprise items related to negative thinking.

Instruments evaluating relevant aspects of negative thinking include, for example, the State Shame and Guilt Scale (SSGS), the Positive and Negative Affect Schedule-Expanded Form (PANAS-X) or the State Hope Scale (SHS).

The State Shame and Guilt Scale (SSGS) is a self-rating scale of in-the-moment (state) feelings of shame, and guilt experiences. It comprises two subscales, a shame and a guilt subscale. The shame subscale comprises items 1, 3, 5, 7, 9. The guilt subscale comprises items 2, 4, 6, 8, 10. All items are scored in a positive direction and are rated on a 5-point Likert scale. It contains some statements which may or may not describe how the patient is feeling right now. A higher score indicates a more intense feeling of shame or guilt.

The Positive and Negative Affect Schedule-Expanded Form (PANAS-X) is a 60-item, expanded version of the PANAS. The PANAS-X measures 11 specific affects: Fear, Sadness, Guilt, Hostility, Shyness, Fatigue, Surprise, Joviality, Self-Assurance, Attentiveness, and Serenity. The PANAS-X thus provides for mood measurement at two different levels. The basic negative emotion scales are fear, hostility, guilt and sadness, while the scale guilt encompasses six items: guilty, ashamed, blameworthy, angry at self, disgusted with self, dissatisfied with self. Each answer should be scored as 1=very slightly or not at all; 2=a little; 3=moderately; 4=quite a bit; or 5=extremely. However, investigators facing more severe time constraints can select and assess only those scales that are most relevant to their research.

More intense feelings of guilt are reflected by a higher score on the guilt scale.

The PANAS-X is simple and easy to administer. Most subjects complete the entire 60-item schedule in 10 minutes or less. This scale consists of a number of words and phrases that describe different feelings and emotions. While it should be indicated to what extent the patient has felt this way during the past few weeks, it has been found that the trait scores on the PANAS-X scales are stable over time, including “at the present moment”, “today” and during “the past few days”, indicating that an appropriate shorter recall period can be applied.

The State Hope Scale (SHS) has three agency and three pathways items to which respondents describe themselves in terms of how they are “right now.” The agency subscale score is derived by summing items 2, 4 and 6, relating to the perceived capacity to use one's pathways to reach desired goals; the pathways subscale score is derived by adding the items 1, 3 and 5, relating to thinking that is used to identify possible ways to achieve a goal. The total State Hope Scale score is derived by summing the three agency and the three pathways items. Scores can range from a low of 6 to a high of 48, wherein higher hope is reflected by a higher score on this scale.

Negative thinking or aspects thereof are also reflected in other scales such as HAM-D, the MADRS, the BPRS or the BDRS, wherein relevant items thereof may be commonly applicable for assessing negative thinking or aspects thereof.

### Scales to Assess Mental and Nervous System Disorders

Numerous scales have been suggested to assess severity of a mental disorder or a nervous system disorder. Such scales are based on tests which can be self-administered or administered by a clinician.

Scales which may be used according to the invention include those known in the art for diagnosis and/or monitoring the mental or nervous system disorders discussed in more detail below.

Treatment outcome is assessed by using one or more indices or scales at one or more time points after completion of a treatment course.

The assessment can be carried out after the acute psychedelic experience has subsided.

An appropriate point in time for an early assessment is generally about 2 to 3 hours after the last administration. An early assessment can generally be carried out, for instance, about 2 hours or about 3 hours after the last administration.

An assessment of an effect on sleep disturbance can, however, be carried out at the earliest on the day after the treatment (i.e., on day 1) so that the treated patient had the opportunity to sleep for at least one night.

Thus, an assessment at day 1 or on day 1 means an assessment on the day following the administration. The assessment will be carried out not earlier than 12 hours after the last administration and in any event not earlier than one night after the last administration and not later than 36 hours after the last administration. The assessment can be carried out after about 24 hours.

An assessment at day 7 or on day 7 means an assessment on the seventh day following the administration (the day of administration is day 0). Analogous definitions apply for other assessment timings measured in days.

When assessing a clinical response, for instance, using one of the scales to assess severity of a mental disorder or a nervous system disorder, at an early timepoint after drug administration (e.g. at 2 hours) based on endpoints which have been developed for a longer recall period (e.g. normally 7 days for the MADRS), a rational modification of such endpoint (e.g. changing the MADRS recall period to 2 hours and carrying forward the sleep item recorded at baseline before drug administration) may be applied. The same applies with respect to any other scale applied herein, unless a recall period is specifically indicated.

The considerations outlined apply for early timepoints because, on the one hand, in order to assess a clinical response, the influence of the patient's status before the treatment on any score recorded after treatment should be kept as low as possible, whereas on the other hand the sleep item cannot be assessed 2 hours after drug administration.

At later timepoints, for instance, on day 1 or later, typically all items of the relevant scales to assess a clinical response can be assessed, using, if necessary, an adapted recall period, so that it is not necessary to carry forward any pre-treatment score.

### Resting State Networks and Negative Thinking

Brain processes can be studied by functional magnetic resonance imaging (fMRI). Brain activity is associated with blood flow, and temporal correlations of spontaneous blood oxygen level dependent (BOLD) signal fluctuations between different brain areas can be measured.

Functional images of the brain are acquired over the course of several minutes. Patterns of low-frequency BOLD signal oscillation are observed across the brain. The decomposition of this spontaneous signal reveals distributed areas with correlated and anti-correlated fluctuations.

In this way, resting-state fMRI can be used to characterize large-scale functional networks, so-called resting-state networks (RSN), which are a set of spatially distinct brain regions that show coordinated activity in the absence of any explicit cognitive task (i.e., at rest). The observed patterns, characterizing a network of brain regions with coherent patterns of signal variation, are called resting-state networks (RSN).

Different resting state networks have been identified and named mostly on the basis of spatial similarity between the resting state networks and activation patterns seen in task fMRI experiments.

Resting-state fMRI can therefore be used to assess the intrinsic functional organization of the brain. Resting-state networks have been characterized for aspects of attention, memory, cognitive control, default mode, motor, and sensory system.

RSNs have been shown to be responsible for various aspects of complex brain function, and it has been found that these connectivity networks are compromised in various disease states. Such disease states, which include certain forms of negative thinking, are associated with altered functional connectivity within a specific resting state network and/or between one or more regions in one or more additional resting state networks.

Major Depressive Disorder (MDD) is a disorder characterized broadly by a high level of negative emotions, and a lower level of positive emotions. More specifically, lower levels of positive emotions such as hope, meaning higher level of hopelessness, and higher levels of negative emotions such as guilt. MDD has been a focus of study in the field of rs-fMRI indicating that MDD is a disorder characterized by widespread network dysfunction. This dysfunction has been found primarily in networks and areas relating to emotional regulation. These include the Default Mode Network (DMN), salience network, affective network, and the prefrontal cortex. Thus, different aspects of negative thinking can be associated with abnormal resting state networks.

Dysfunctional connectivity in resting state networks has been also reported for patients with repetitive negative thinking (RNT) including an altered connectivity of the left Executive Control Network and the Anterior Salience Network with the ventral Default Mode Network.

Thus, patients suffering from negative thinking show altered functional connectivity within and/or between resting state networks when compared to healthy, age-matched controls. Alterations are observed within and/or between at least the default mode network, the executive control network, the salience network, the affective network, and the prefrontal cortex.

In many instances, RSNs involved in negative thinking are affected by mental or nervous system disorders, such as disorders characterized by depressive episodes, for example, Major Depressive Disorder (MDD), Bipolar Disorder (BD), such as Bipolar I Disorder and Bipolar II Disorder, Postpartum Depression (PPD), Seasonal Affective Disorder and Persistent Depressive Disorder; Anxiety Disorder, for example Generalised Anxiety Disorder (GAD) and Social Anxiety Disorder (SAD); Obsessive Compulsive and Related Disorders, for example, Obsessive Compulsive Disorder (OCD) and Body Dysmorphic Disorder (BDD); Post-Traumatic Stress Disorder (PTSD); Pain Disorders, for example, Chronic Pain; Mental and Behavioural Disorders due to Psychoactive Substance Use, for example Substance Use Disorder (SUD); Psychotic Disorder, for example Schizophrenia; Dementia, for example Alzheimer's Dementia (AD); Eating Disorders; Attention Deficit Hyperactivity Disorder (ADHD); Personality Disorders, for example Schizotypal Personality Disorder and Borderline Personality Disorder (BPD).

Resting state networks involved in negative thinking are also affected by mental or nervous system conditions which are a consequence of certain medical health conditions, such as Traumatic Brain Injury (TBI).

Resting state networks involved in negative thinking are also affected by sleep disturbance, for instance, insomnia. In fact, negative thinking and impairment of sleep are correlated.

### Treatment of Negative Thinking and Mental or Nervous System Disorders

According to the invention, negative thinking occurring in a patient suffering from a mental disorder or a nervous system disorder can be treated. Moreover, negative thinking occurring in a patient suffering from sleep disturbance, for instance, insomnia, can be treated.

In patients suffering from negative thinking in association with another condition as detailed above, a treatment of negative thinking according to the invention leads to an improvement of the condition with which the negative thinking is associated.

Treatment according to the invention is by administration of 5-MeO-DMT or a pharmaceutically acceptable salt thereof.

5-MeO-DMT administered to a patient disrupts established functional connectivity patterns within and/or between resting state networks. This disruption leads to a reset of the pathological ill-connected connections as the networks reconnect. New, healthy functional connections are established with persistent effects.

Thus, according to the invention, influencing those networks by a therapy as described herein will lead to an improvement of the negative thinking and, if the patient treated suffers from a mental disorder or a nervous system disorder, also of that disorder; if the patient treated suffers from sleep disturbance, for instance, insomnia, also of the sleep disturbance, for instance, insomnia.

To further support the clinical application of 5-MeO-DMT in patients suffering from negative thinking, the inventors assessed clinical data relating to the use of 5-MeO-DMT in patients treated because of mental disease and noted particular improvements in negative thinking which is typically also observed in patients with other disorders.

The data stem from a recently completed clinical trial investigating the use of 5-MeO-DMT in the treatment of patients diagnosed with Treatment Resistant Depression (TRD; see also the examples section below). While TRD is a specific condition, the inventors determined, as discussed in detail below, that certain clinical observations are made in the trial are relevant for devising a treatment for other conditions which are associated with negative thinking.

In the clinical trial, 5-MeO-DMT was administered via inhalation (as described in more detail in the example section below). Patients were assigned to different groups. In the context of the present invention, the group who received a single, 12 mg dose and the group who underwent an intra-day individualized dosing regimen (IDR) that allowed for multiple, escalating doses (6 mg, 12 mg and 18 mg) within a single day, driven by the intensity of the patient-reported psychedelic experience are of interest.

The data gathered include the assessment of the treated patients against several scales including the Montgomery-Åsberg Depression Rating Scale (MADRS) and the Brief Psychiatric Rating Scale (BPRS). While the focus of the trial was on demonstrating treatment efficacy through improvements in overall MADRS score, the inventors focused on the items comprising the various rating scales and noticed that particular subscore items, like items related to negative thinking, are relevant for other conditions in which negative thinking is based on similarly altered functional connectivity within and/or between the default mode network, the executive control network, the salience network, the affective network, and the prefrontal cortex.

Multiple patients within the recruited cohort displayed significant improvements, a result that confirms the inventors' finding that 5-MeO-DMT is a compound suitable for treating patients presenting with these symptoms.

More in particular, an aspect which can be treated by administration of 5-MeO-DMT, is negative thinking, in particular feelings of worthlessness, helplessness and hopelessness, and/or guilt. 5-MeO-DMT can be administered to patients to reduce or eliminate negative thinking, in particular feelings of worthlessness, helplessness and hopelessness, and/or guilt, in said patients.

The MADRS scale item that is of particular relevance to negative thinking is “pessimistic thoughts”, which represents thoughts of guilt, inferiority, self-reproach, sinfulness, remorse and ruin.

A score of 0 is assigned if there are no pessimistic thoughts. The score is 2 in the case of fluctuating ideas of failure, self-reproach or self-depreciation. A score means persistent self-accusations, or definite but still rational ideas of guilt or sin as well as the patient being increasingly pessimistic about the future. A score of 6 is assigned in the case of delusions of ruin, remorse or unredeemable sin and self-accusations which are absurd and unshakable.

In the study group receiving the individualized dosing regimen, the aggregated score for the MADRS item “pessimistic thoughts” across all 8 patients was 28 at base line.

After 2 hours, it was reduced to 7 which corresponds to an improvement of 21 points or 75%. At day 1 after treatment, it was reduced to 4 which corresponds to an improvement of 24 points or 86%. At day 7 after treatment, it was reduced to 3 which corresponds to an improvement of 25 points or 89%.

In the 12 mg group, the aggregated score for the MADRS item “pessimistic thoughts” across all 4 patients was 16 at base line. After 2 hours, it was reduced to 8 which corresponds to an improvement of 8 points or 50%. At day 1 after treatment, it was reduced to 7 which corresponds to an improvement of 9 points or 56%.

At day 7 after treatment, it was reduced to 8 which corresponds to an improvement of 8 points or 50%.

The BPRS item that is of particular relevance to negative thinking is “guilt feelings”. This item relates to over concern or remorse for past behaviour. Possible scores are:
1—No guilt feelings.
2—Very Mild. Concerned about having failed someone or at something but not preoccupied. Can shift thoughts to other matters easily.
3—Mild. Concerned about having failed someone or at something with some preoccupation. Tends to voice guilt to others.
4—Moderate. Disproportionate preoccupation with guilt, having done wrong, injured others by doing or failing to do something, but can readily turn attention to other things.
5—Moderately Severe. Preoccupation with guilt, having failed someone or at something, can turn attention to other things, but only with great effort. Not delusional.
6—Severe; Delusional guilt or unreasonable self-reproach very out of proportion to circumstances. Moderate preoccupation present.
7—Extremely Severe. Delusional guilt or unreasonable self-reproach grossly out of proportion to circumstances. Subject is very preoccupied with guilt and is likely to disclose to others or act on delusions.

In the study group receiving the individualized dosing regimen, the aggregated score for the BPRS item “guilt feelings” across all 8 patients was 34 at base line.

After 3 hours, it was reduced to 14 which corresponds to an improvement of 20 points or 59%. At day 1 after treatment, it was reduced to 11 which corresponds to an improvement of 23 points or 68%. At day 7 after treatment, it was reduced to 10 which corresponds to an improvement of 24 points or 71%.

In the 12 mg group, the aggregated score for the BPRS item “guilt feelings” across all 4 patients was 18 at base line.

After 3 hours, it was reduced to 9 which corresponds to an improvement of 9 points or 50%. At day 1 after treatment, it was reduced to 5 which corresponds to an improvement of 13 points or 72%. At day 7 after treatment, it was reduced to 5 which corresponds to an improvement of 13 points or 72%.

Thus, the score of the MADRS scale item that is of particular relevance to negative thinking, “pessimistic thoughts”, is markedly improved, as is the score of the BPRS item “guilt feelings”. The inventors conclude that 5-MeO-DMT can be used to treat negative thinking in patients, in particular patients also suffering from a mental disorder or a nervous system disorder or a sleep disturbance, for instance insomnia.

Consequently, according to the invention, the treatment of a patient suffering from negative thinking with 5-MeO-DMT or a pharmaceutically acceptable salt thereof reduces or eliminates negative thinking.

### The Active Agent

The above discussion shows that negative thinking as such present a significant disease burden and deserve appropriate treatment.

The inventors considered that a carefully chosen hallucinogen may lead to an improved treatment of important aspects of negative thinking and may lead to overall improvements of the condition.

One group of hallucinogens entails compounds which bind to the 5-hydroxytryptamine (5-HT) receptors, which are also referred to as serotonin receptors (described are 7 families 5-HT1 to 5-HT7 with several subtypes). Examples are lysergic acid diethylamide (LSD), psilocybin, and N,N-dimethyltryptamine (DMT). These serotonergic agents are often referred to as “psychedelics”, which emphasizes their predominant ability to induce qualitatively altered states of consciousness such as euphoria, trance, transcendence of time and space, spiritual experiences, dissolution of self-boundaries, or even near-death experiences, while other effects such as sedation, narcosis, or excessive stimulation are only minimal.

Chemically, serotonergic psychedelics are either phenylalkylamines or indoleamines, with the indoleamine class being divided into two subsets, ergolines and tryptamines, the latter being derived from tryptamine.

The various serotonergic psychedelics have different binding affinity and activation potency for various serotonin receptors, particularly 5-HT1A, 5-HT2A, and 5-HT2C, and their activity may also be modulated by interaction with other targets such as monoamine transporters and trace amine-associated receptors.

Recently published clinical studies which have used serotonergic psychedelic drugs such as LSD, psilocybin and DMT (using the shamanic brew Ayahuasca, which contains DMT) in certain mental disorders suggest that those compounds could provide an alternative to the currently available treatments for certain mental disorders. However, there are reports that these compounds can induce mania in patients suffering from depressive symptoms, and this may preclude their clinical use.

For instance, Lake et al. (Lake, C. R., Stirba, A. L., Kinneman, R. E. Jr, Carlson, B., Holloway, H. C., 1981. Mania associated with LSD ingestion. American Journal of Psychiatry. 138(11):1508-9) report about a patient who suffered a manic attack after ingesting LSD or an LSD analogue. The patient experienced acute symptoms of LSD intoxication, which resolved but were followed in about 3 weeks by a typical manic episode of psychotic magnitude. Hendin and Penn (Hendin, H. M., Penn, A. D., 2021. An episode of mania following self-reported ingestion of psilocybin mushrooms in a woman previously not diagnosed with bipolar disorder: A case report. Bipolar Disorders 23(4):1-3) report about an episode of mania following self-reported ingestion of psilocybin mushrooms. Szmulewicz et al. (Szmulewicz, A. G., Valerio, M. P., and Jose M Smith, J. M., 2015. Switch to mania after ayahuasca consumption in a man with bipolar disorder: a case report. International Journal of Bipolar Disorders (2015) 3:4) report on a switch to mania after consumption of ayahuasca, a DMT containing brew, in a man with bipolar disorder.

A further case report is found in Brown, T., Shao, W., Ayub, S., Chong, D., & Cornelius, C. (2017). A Physician's attempt to self-medicate bipolar depression with N, N-dimethyltryptamine (DMT). Journal of Psychoactive Drugs, 49(4), 294-296.

The inventors considered that in order to avoid the induction of mania or hypomania or at least reduce the risk of induction of mania or hypomania, the compound administered must be appropriately chosen and preferably is administered in a particular dosing regimen.

The inventors identified 5-methoxy-N,N-dimethyltryptamine (5-MeO-DMT) as a psychedelic of particular interest for use in therapy. 5-MeO-DMT has a distinct pharmacological profile which differs from that of other psychedelic compounds.

5-MeO-DMT is a potent, fast-acting, naturally occurring serotonin (5-HT) agonist, acting at both the 5-HT1A and the 5-HT2A receptor, with higher affinity for the 5-HT1A receptor subtype compared to other classical psychedelics.

Inhibition constants (Kᵢ values) as further detailed on the example section below for psilocin (the dephosphorylated from of psilocybin which is formed after uptake of psilocybin), DMT and 5-MeO-DMT are 48, 38 and 1.80 nM, respectively, at 5-HT1A receptors located in the hippocampus of post-mortem human brain. Thus, 5-MeO-DMT exhibits high affinity and psilocin and DMT exhibit moderate affinity for 5-HT1A receptors. Inhibition constants (Kᵢ values) for psilocin, DMT and 5-MeO-DMT are 37, 117 and 122 nM, respectively, at 5-HT2A receptors located in the frontal cortex of post-mortem human brain. Therefore, psilocin exhibits moderate/strong affinity and DMT and 5-MeO-DMT exhibit comparatively weak affinity for 5-HT2A receptors.

Relative to the other psychoactive compounds mentioned previously, 5-MeO-DMT displays an enhanced affinity for the 5-HT1A receptor, where it acts as a potent agonist. In the case of psilocin and DMT, there is an increased contribution of 5-HT2A binding, relative to 5-MeO-DMT, with the latter displaying the largest differential affinity for 5-HT1A over 5-HT2A of the three compounds. Therefore, 5-HT1A binding plays a much bigger role in the overall effect of 5-MeO-DMT relative to 5-HT2A binding compared to the other two compounds.

It has been reported that 5-HT1A agonism reduces impulsivity and aggression, whereas 5-HT2A agonism can result in short-term increases in these same traits. Furthermore, the dopamine system has been implicated in contributing to mania, with increased dopamine drive being linked to mania. LSD, psilocybin and DMT all display increased affinity for a variety of dopamine receptors relative to 5-MeO-DMT

Compared to other psychedelics, like LSD, psylocibin or DMT, 5-MeO-DMT can be administered to patients, preferably using dosing schemes as described herein, without a significant risk of inducing mania or hypomania in a patient suffering from a mental or nervous system disorder, including a disorder characterized by depressive episodes, for example, Major Depressive Disorder (MDD), Postpartum Depression (PPD), Persistent Depressive Disorder, Seasonal Affective Disorder and Bipolar Disorder (BD), such as Bipolar I Disorder and Bipolar II Disorder; a Psychotic Disorder, such as Schizophrenia; or a personality disorder, such as Schizotypal Personality Disorder. The patient suffering from such a mental or nervous system disorder, treated according to the invention, does not experience treatment-emergent mania or hypomania.

It is also noted that reports of treatment-emergent mania or hypomania related to psychoactive substance use seem to indicate large quantities of the respective compounds (e.g., DMT/ayahuasca, psilocybin, LSD) were used.

The inventors' approach of sequential up-titration of 5-MeO-DMT significantly reduces the risk of excessive dose administration with its potential for attendant adverse events.

Still further, the induction by antidepressants of isolated events of hypomania has been reported in patients suffering from treatment resistant depression (TRD) (Bader, Cynthia D., and David L. Dunner. “Antidepressant-induced hypomania in treatment-resistant depression.”⋅Journal of Psychiatric Practice⋅13.4 (2007): 233-237). However, the recently concluded clinical trial of 5-MeO-DMT in TRD patients showed no evidence of hypomania induction.

5-MeO-DMT can induce peak experiences, i.e., experiences characterized by an emotional perspective shift, which is described as “loss of ego” which often culminates in an overwhelming sense of “oneness with the universe”, more rapidly than other psychedelics and has a short duration of acute psychedelic effects (5 to 30 minutes after inhalation compared with several hours for e.g. oral psilocybin and oral LSD). These characteristics of 5-MeO-DMT are associated with an improved therapeutic profile which can be explained by specific alterations of Resting State Network (RSN) activity under 5-MeO-DMT treatment.

Furthermore, 5-MeO-DMT is a 5-HT7 receptor agonist showing high affinity towards the receptor. The inventors determined, using recombinant human 5-HT7 receptor, [³H]LSD as a radio ligand and serotonin to estimate non-specific binding, a Kᵢ of 2.3 nM.

Thus, besides the 5-HT1A and 5-HT2A receptors discussed above, 5-MeO-DMT also interacts with the 5-HT7 receptor. 5-MeO-DMT act as an agonist on this receptor and shows a high (nanomolar) binding affinity.

The 5-HT7 receptor has a role in neurogenesis, synaptogenesis and dendritic spine formation. It is, among other things, associated with central processes such as learning and memory, with sleep regulation and circadian rhythm and with nociception.

The 5-HT7 receptor is in particular expressed in the spinal cord, raphe nuclei, thalamus, hypothalamus including the suprachiasmatic nucleus, hippocampus, prefrontal cortex, striatal complex, amygdala and in the Purkinje neurons of the cerebellum.

The suprachiasmatic nucleus is the central pacemaker of the circadian timing system. It coordinates circadian rhythms in various brain regions. Disruption of this coordination will result in disease states, in particular disease states involving sleep disturbance. In patients suffering from sleep disturbance resting state functional connectivity analysis reveals alterations in functional connectivity between the suprachiasmatic nucleus and regions within the default mode network.

The expression of the 5-HT7 receptor in the suprachiasmatic nucleus corresponds to the function of the receptor in regulation of sleep/wake cycles. The inventors consider that this allows treatment of patients suffering from sleep disturbance by 5-MeO-DMT which acts on the receptor.

The inventors consider that binding of 5-MeO-DMT to the 5-HT7 receptor as one mediator of the pharmacological effects of 5-MeO-DMT, which involve functional connectivity “resets” of networks and neuroplasticity effects, contributes to the beneficial effects of 5-MeO-DMT in the treatment of patients suffering from sleep disturbance.

The inventors further consider that binding of 5-MeO-DMT to the 5-HT7 receptor as well as to the 5-HT1A receptor as two mediators of effects exerted by 5-MeO-DMT, which include functional connectivity “resets” of networks and neuroplasticity effects, allows achieving beneficial effects also in patients suffering from other symptoms or conditions, such as cognitive dysfunction, anxiety, psychomotor retardation, sleep disturbance or social/emotional withdrawal. This is supported by the clinical results demonstrated in studies referred to herein.

Another feature of 5-MeO-DMT is its short half-life. 5-MeO-DMT is mainly inactivated through a deamination pathway mediated by monoamine oxidase A, and it is O-demethylated by cytochrome P450 2D6 (CYP2D6) enzyme.

The inventors investigated pharmacokinetic properties of 5-MeO-DMT and observed rapid absorption and distribution of inhaled 5-MeO-DMT, with maximum concentrations and pharmacological effects observed during and immediately after dosing.

An analysis of the pharmacokinetic properties of 5-MeO-DMT after inhalation shows a very rapid decline of the plasma concentration. Already 10 minutes after administration, the concentration drops to 10% of Cmax or below; after 2 hours, it is 1% of Cmax or below; after 3 hours, 5-MeO-DMT is no longer detectable in the plasma. This applies over the whole dose range tested (6 mg, 12 mg, 18 mg). No accumulation is observed upon repeated administration within a time frame of 1 to 4 hours. Uptitration as disclosed herein will not lead to accumulation and thus not to higher plasma concentrations, for instance, 10 minutes, 2 hours, or 3 hours after administration.

The properties of 5-MeO-DMT make the compound especially suitable for the treatment of negative thinking, in particular for patients suffering from a mental disorder or a nervous system disorder.

The properties of 5-MeO-DMT also allow specific dosage regimens, as discussed in more detail below.

According to the invention, isotopic variants of 5-MeO-DMT and pharmaceutically acceptable salts thereof can also be used. When reference is made to the use of 5-MeO-DMT or a pharmaceutically acceptable salt thereof, the use of isotopic variants is also contemplated.

These variants are in particular deuterated forms of 5-MeO-DMT and pharmaceutically acceptable salts of such forms.

Deuterated forms of 5-MeO-DMT are forms having a higher deuterium content than expected based on the natural abundance of this isotope.

Deuterated forms of 5-MeO-DMT are in particular forms wherein deuterium has been introduced at one or more defined hydrogen positions.

Examples of deuterated forms of 5-MeO-DMT include, without limitation, 1-deuterio-2-(5-methoxy-1H-indol-3-yl)-N,N-dimethylethanamine, 1,1-dideuterio-2-(5-methoxy-1H-indol-3-yl)-N,N-dimethylethanamine, 1,1,2,2-tetradeuterio-2-(5-methoxy-1H-indol-3-yl)N,N-dimethylethanamine, and N,N-dimethyl-2-[5-(trideuteriomethoxy)-1H-indol-3-yl]ethanamine.

Further examples include forms of 5-MeO-DMT wherein deuterium has been introduced at one or more hydrogen positions of the N-bound methyl groups. Still further examples include forms of 5-MeO-DMT wherein one or more deuterium atoms replace hydrogen atoms of the indole ring system. It is moreover noted that combinations of the above substitution patterns are also contemplated.

Preparation methods for these compounds are known in the art.

According to the invention, mixtures of deuterated forms of 5-MeO-DMT, mixtures of one or more deuterated form with non-deuterated 5-MeO-DMT, pharmaceutically acceptable salts of deuterated forms of 5-MeO-DMT, mixture of such salts as well as mixtures of salts of deuterated and non-deuterated 5-MeO-DMT can also be used.

Further according to the invention, deuterated 5-MeO-DMT and salts of deuterated 5-MeO-DMT are used in amounts that are equimolar to the amounts of the corresponding non-deuterated forms.

According to the invention, prodrugs of 5-MeO-DMT and pharmaceutically acceptable salts of such prodrugs can also be used. Such prodrugs of 5-MeO-DMT can be metabolically converted to 5-MeO-DMT. Thus, when reference is made to the use of 5-MeO-DMT or a pharmaceutically acceptable salt thereof, this can be replaced by a 5-MeO-DMT prodrug or a salt thereof.

In suitable prodrugs, the hydrogen in position 1 of the indole moiety is substituted by an organic moiety which can be split off after administration.

Examples of suitable organic moieties are —C(O)OR¹, —C(O)R², —CH(R³)OR⁴, —C(O)OCH(R³)OC(O)R⁴, —C(O)OCH(R³)OC(O)OR⁴, —CH(R³)C(O)R⁴, —CH(R³)OC(O)R⁴, —CH(R³)OC(O)OR⁴, wherein each of R¹, R², R³, and R⁴ is independently hydrogen, alkyl, heteroalkyl, cycloalkyl, heterocyclylalkyl, aryl, or heteroaryl, wherein each alkyl, heteroalkyl, cycloalkyl, heterocyclylalkyl, aryl, and heteroaryl is independently substituted or unsubstituted.

Preferred examples of organic moieties are —CH(R³)OC(O)R⁴ and —C(O)OR¹, wherein R¹, R³, and R⁴ are defined as above.

Prodrugs, especially those of the above structure, can also be used on the form of pharmaceutically acceptable salts.

Specific examples of prodrugs are 5-MeO-DMT carboxy-isopropyl valinate, preferably in salt form, in particular as ditrifluoroacetate (1-(((S)-2-amino-3-methylbutanoyl)oxy)-2-methylpropyl 3-(2-(dimethylamino)ethyl)-5-methoxy-1H-indole-1-carboxylate di-trifluoroacetate) and 5-MeO-DMT methyl pivalate (3-(2-(dimethylamino)ethyl)-5-rnethoxy-1H-indol-1-yl)methyl pivalate).

Preparation methods for prodrugs as discussed herein are known in the art.

According to the invention, the Tₘₐₓ value of the metabolite 5-MeO-DMT as measured in male Sprague-Dawley (SD) rats following oral dosing of the prodrug at 10 mg/kg is preferably 1 hour or less, more preferably 0.7 hours or less and in particular 0.5 hours or less.

Further according to the invention, prodrugs of 5-MeO-DMT and salts of prodrugs of 5-MeO-DMT are used in amounts that are equimolar to the amounts of the corresponding non-prodrug forms.

### Modes of Administration

The therapeutically effective amount of 5-MeO-DMT is administered by inhalation, by nasal administration, by buccal administration or by sublingual administration. Administration via these routes can assure a rapid onset of action. A most preferred route of administration is administration by inhalation. Preferably, the inhalation of the therapeutically effective amount of 5-MeO-DMT occurs within a single breath.

For nasal administration, 5-MeO-DMT can be employed as a neat substance or in the form of a formulation for nasal administration, examples of which are known in the art.

For nasal administration, 5-MeO-DMT can be employed as a pharmaceutically acceptable salt, preferably the hydrobromide salt, or in the form of a formulation of a pharmaceutically acceptable salt, preferable the hydrobromide salt. Examples of appropriate devices are known in the art.

Buccal administration or sublingual administration can also rely on a pharmaceutically acceptable salt of 5-MeO-DMT, preferable the hydrobromide salt, as such or in the form of formulations, for instance, tablets, films, sprays, creams, as generally known in the art.

Administration is in particular by inhalation of an aerosol. Such an aerosol comprises (a) a pharmaceutically acceptable gas; (b) aerosol particles of 5-methoxy-N,N-dimethyltryptamine (5-MeO-DMT) or a pharmaceutically acceptable salt thereof, wherein the aerosol has an aerosol particle mass density of about 0.5 mg/l to about 18 mg/I, such as about 0.5 mg/l to about 12.5 mg/I, preferably of about 1.3 mg/l to about 10 mg/I, in particular of about 2 mg/l to about 9 mg/I. The pharmaceutically acceptable gas is preferably air.

The aerosol particles preferably contain less than 1 wt % impurities, in particular less than 0.5 wt % impurities. They furthermore preferably contain less than 0.5 wt % 5-MeO-DMT degradation products, in particular less than 0.2 wt % 5-MeO-DMT degradation products resulting from a chemical modification of 5-MeO-DMT as a result of a chemical reaction during aerosol formation.

In a further preferred aspect, the aerosol essentially consists of (a) air; (b) aerosol particles of 5-MeO-DMT or a pharmaceutically acceptable salt thereof.

The aerosol particles preferably contain 5-MeO-DMT in the form of the free base.

The aerosol is preferably characterized by a mass median aerodynamic diameter of less than 3 μm and more than 0.1 μm, in particular by a mass median aerodynamic diameter of less than 2 μm and more than 0.1 μm.

The aerosol may be formed by a) exposing a thin layer of 5-MeO-DMT or a pharmaceutically acceptable salt thereof, configured on a solid support, to thermal energy, and b) passing air over the thin layer of 5-MeO-DMT to produce aerosol particles. The thin layer may have a thickness of less than about 10 μm, in particular less than about 7.5 μm. It may have a thickness in the range of about 0.1 μm to about 10 μm, in particular in the range of about 0.3 μm to about 7.5 μm.

The thin layer of 5-MeO-DMT, configured on a solid support, may be exposed to thermal energy via the air passing over the thin layer. Alternatively, the thin layer of 5-MeO-DMT, configured on a solid support, may be exposed to thermal energy via the solid support.

The air passing over the thin layer may have a temperature in the range of about 180° C. to about 260° C. The air passing over the thin layer may in particular have a temperature of about 210° C. and pass over the thin layer at a rate of about 12 l/min for a duration of about 15 seconds.

The aerosol particles may be contained in a volume of equal or less than about 3 liters, in particular in a volume of about 1 to about 3 liters, such as about 2 to about 3 liters. It is preferably delivered to a patient via a single inhalation.

5-MeO-DMT or a pharmaceutically acceptable salt thereof is provided in a form suitable for inhalation in a medical context. 5-MeO-DMT and pharmaceutically acceptable salts thereof are provided in the form of aerosols. These aerosols have a suitable aerosol particle mass density so that a therapeutically effective dose of the aerosol can be administered to a patient via a single inhalation.

Aerosols useful in the present invention can be formed using thermal energy. When using thermal energy to form an aerosol of a compound, it is very difficult to predict which conditions are suitable for safe, efficient and predictable aerosolization, in particular if the aerosol is to be used for systemic delivery of that compound to a patient via the lungs.

Relevant variables in this context include a) the dose of the compound, b) the morphological state in which that compound is made available for aerosolization (e.g. in crystal form, or in form as a thin layer), c) the amount of thermal energy to which the compound is exposed (defined by temperature and duration of exposure), and d) the volume of air introduced to create the aerosol (defined by flow rate and duration of air flow).

The compositions and methods described herein are for safe, efficient and predictable systemic delivery of 5-MeO-DMT or a pharmaceutically acceptable salt thereof to a patient through inhalation. “Safe” means that the aerosol particles should contain only a very small amount of impurities and 5-MeO-DMT degradation products, “efficient” means that the dosage is aerosolized to a defined extent and preferably almost completely or completely, that the aerosol has desirable physical properties for delivery of the 5-MeO-DMT or a pharmaceutically acceptable salt thereof systemically via the lungs mainly via absorption in the pulmonary alveoli, and that the aerosol can be inhaled by the patient in a single inhalation (i.e., within one deep breath), and “predictable” means that there should be almost no or no variability in the amount of degradation products, in the extent of aerosolization, and in the physical properties of the aerosol.

A suitable aerosol can be achieved by a) providing the therapeutically effective amounts of 5-MeO-DMT as a thin layer, on a solid support, b) exposing the thin 5-MeO-DMT layer to elevated controlled temperatures for a short duration of time, and c) providing a controlled amount of air so that an aerosol is formed.

A composition for delivery of a therapeutically effective amount of 5-MeO-DMT may comprise an aerosol, wherein the aerosol is formed by a) exposing a thin layer of 5-MeO-DMT, configured on a solid support, to thermal energy, and b) passing air over the thin layer of 5-MeO-DMT; wherein said aerosol has one or more of the following features: 1) it contains aerosol particles which are characterized by a mass median aerodynamic diameter of less than 3 micron, 2) it contains aerosol particles which are characterized by less than 1% wt impurities and less than 0.5% 5-MeO-DMT degradation products, 3) it can be delivered to a patient via a single inhalation.

The generation of aerosol particles characterized by a mass median aerodynamic diameter of less than 3 microns, with less than 1% wt impurities and less than 0.5% wt 5-MeO-DMT drug degradation products, in an aerosol volume which can be delivered to a patient via a single inhalation, is achieved by defining a) the dosage amount of 5-MeO-DMT contained in the thin layer of 5-MeO-DMT, b) the thickness of the thin layer of the 5-MeO-DMT, c) the thermal energy to which the thin layer of 5-MeO-DMT is exposed (defined by temperature and duration of exposure), and d) the total amount of the air which passes over the thin layer of 5-MeO-DMT (defined by airflow rate and duration of airflow).

Preferably the thin layer of 5-MeO-DMT is exposed to thermal energy via the air passing over the thin layer, in which case that air is heated. The heated air passing over the thin layer may have a temperature in the range of about 180° C. to about 260° C. The air passing over the thin layer may in particular have a temperature of about 210° C.

Alternatively, the thin layer of 5-MeO-DMT is exposed to thermal energy via the solid support, in which case the air passing over the thin layer is not heated, but the solid support is heated. The heated solid support may have a temperature in the range of about 180° C. to about 420° C.

Preferably the 5-MeO-DMT used for formation of the thin layer, on the solid support, is highly pure, with a purity of at least 99%, preferably at least 99.5%.

Preferably the dosage amount of 5-MeO-DMT contained in the thin layer of 5-MeO-DMT, configured on the solid support, is from about 1 mg to about 25 mg, preferably from about 2 mg to about 20 mg, more preferably from about 4 mg to about 20 mg. Useful specific amounts are, e.g., about 4 mg, about 6 mg, about 8 mg, about 10 mg, about 12 mg, about 14 mg, about 16 mg, about 18 mg, and about 20 mg. Preferred specific amounts are e.g. about 6 mg, about 12 mg, and about 18 mg.

Solid supports, on which 5-MeO-DMT or a pharmaceutically acceptable salt thereof is provided, can have a variety of shapes. Examples of such shapes include, without limitation, cylinders of less than 1.0 mm in diameter, boxes of less than 1.0 mm thickness and virtually any shape permeated by small (e.g., less than 1.0 mm-sized) pores. Preferably, solid supports provide a large surface to volume ratio (e.g., greater than 100 per meter) and a large surface to mass ratio (e.g., greater than 1 cm² per gram).

A solid support of one shape can also be transformed into another shape with different properties. For example, a flat sheet of 0.25 mm thickness has a surface to volume ratio of approximately 8,000 per meter. Rolling the sheet into a hollow cylinder of 1 cm diameter produces a support that retains the high surface to mass ratio of the original sheet but has a lower surface to volume ratio (about 400 per meter).

A number of different materials are used to construct the solid supports. Classes of such materials include, without limitation, metals, inorganic materials, carbonaceous materials and polymers. The following are examples of the material classes: aluminum, silver, gold, stainless steel, copper and tungsten; silica, glass, silicon and alumina; graphite, porous carbons, carbon yarns and carbon felts; polytetrafluoroethylene and polyethylene glycol. Combinations of materials and coated variants of materials are used as well.

Where aluminum is used as a solid support, aluminum foil is a suitable material. Examples of silica, alumina and silicon based materials include amphorous silica S-5631 (Sigma, St. Louis, Mo.), BCR171 (an alumina of defined surface area greater than 2 m²/g from Aldrich, St. Louis, Mo.) and a silicon wafer as used in the semiconductor industry. Carbon yams and felts are available from American Kynol, Inc., New York, N.Y.

Preferably the thickness of the thin layer of the 5-MeO-DMT, configured on the solid support, is less than about 10 μm, in particular less than about 7.5 μm. It may have a thickness in the range of about 0.1 μm to about 10 μm, in particular in the range of 0.3 μm to 7.5 μm.

Preferably the total amount of the air passing over the thin layer of 5-MeO-DMT is defined by a flow rate of between about 6 liters per minute and about 40 liters per minute, preferable between about 8 liters per minute and about 16 liters per minute and the duration of airflow is chosen so that the total volume of aerosol does not exceed about 3 liters, preferably is between about 1 liter and 3 liters, such as between 2 liters and 3 liters. E.g., at an airflow rate of about 6 liters per minute, the duration of airflow should be less than about 30 seconds. A useful specific airflow rate and duration is about 12 liters per minute and about 15 seconds, leading to an aerosol volume of about 3 liters. Another useful specific airflow rate and duration is 10 liters per minute and about 15 seconds, leading to leading to an aerosol volume of about 2.5 liters. Another useful specific airflow rate and duration is 8 liters per minute and about 15 seconds, leading to leading to an aerosol volume of about 2 liters. Another useful specific airflow rate and duration is 10 liters per minute and about 12 seconds, leading to leading to an aerosol volume of about 2 liters.

The aerosol formation rate is greater than 0.1 mg/sec.

The aerosol has an aerosol particle mass density of about 0.5 mg/l to about 18 mg/I, such as of about 0.5 mg/l to about 12.5 mg/I, preferably of about 1.3 mg/l to about 10 mg/I, in particular of about 2 mg/l to about 9 mg/I.

The 5-MeO-DMT aerosol particles are characterized by a mass median aerodynamic diameter of less than 3 micron and more than 0.1 micron, preferably of less than 2.5 micron and more than 0.1 micron, most preferably of less than 2 micron and more than 0.1 micron. The 5-MeO-DMT aerosol particles are characterized by less than 1% wt impurities, preferably by less than 0.5% wt impurities.

The 5-MeO-DMT aerosol particles are characterized by less than 0.5% wt 5-MeO-DMT degradation products, preferably by less than 0.2% wt 5-MeO-DMT degradation products.

A composition for delivery of a therapeutically effective amount of 5-MeO-DMT may comprise an aerosol, wherein the aerosol is formed by a) exposing a dosage amount of 12 mg 5-MeO-DMT, configured as a thin layer of less than 5 micron thickness on a solid support, to a temperature of 210° C. via passing heated air over the thin layer for a duration of 15 seconds; wherein said aerosol has one or more of the following features: 1) it contains aerosol particles which are characterized by a mass median aerodynamic diameter of less than 3 micron, 2) it contains aerosol particles which are characterized by less than 1% impurities and less than 0.5% wt 5-MeO-DMT degradation products, 3) it can be delivered to a patient via a single inhalation.

A skilled person, knowing the aerosol characteristics and the aerosolization conditions defined in the present invention, can identify suitable vaporization devices or systems, which lead to the required aerosol characteristics. Examples of such suitable vaporization devices or systems include e.g. the Volcano Medic Vaporization System with the associated dosing capsules with drip pad (Storz & Bickel, Germany; as disclosed in e.g. EP 0 933 093 B1, and EP 1 884 254 B1 and Registered Community Design 003387299-0001) and the Staccato device (Alexza Pharmaceuticals, Mountain View, USA; as disclosed e.g. in U.S. Pat. No. 7,458,374 B2, U.S. Pat. No. 9,370,629 B2 and U.S. Pat. No. 9,687,487 B2). The aerosol generated may be collected in a balloon and inhaled by the patient from the balloon.

### Dosing Regimen

The present invention also provides dose ranges, particular doses as well as dosing regimens (administration schemes).

The invention is in part based on the inventors' conclusion that the occurrence of a peak psychedelic experience during the acute phase after administration of 5-MeO-DMT or a pharmaceutically acceptable salt thereof is driving its therapeutic benefit in patients suffering from negative thinking, in particular one or more of the aspects defined above, either in a causal relationship or at least as a surrogate behavioural marker for the underlying unknown therapeutic mechanism.

Consequently, achieving peak experiences more rapidly, in a larger proportion of patients and with better reproducibility in an individual patient, compared with previously tested psychedelic agents and dosing regimens, will lead to a better therapeutic profile.

Further, the present invention also relies on the short duration of action of 5-MeO-DMT and the absence of relevant tolerance (i.e., the absence of diminished or no psychedelic effects after re-administration), as a basis for enabling a dosing regimen with frequent re-administrations (such as more than once daily, or daily), which are designed to increase the rate of occurrence of peak experiences, thereby increasing the therapeutic benefit. Such repeat administrations within short time also allow an intraindividual dose-optimization which reduces the risk of overdosing, which may otherwise lead to somatic side effects, such as the serotonin syndrome, negative psychic reactions, such as flashbacks of the experience at later timepoints, induction of mania or hypomania or to less meaningful psychedelic experiences with few or no memories of the altered state (so-called “white-outs”). Further, starting with a low dose allows familiarization of the patient with the psychedelic experience in general, and allows preparation for the more intense symptoms to occur at the higher doses, which will positively influence the experience at those higher doses. Also, the prospect of being able to initiate treatment with a low dose will increase patient acceptance of the therapeutic approach and improve overall compliance rates on the patient population level.

Frequent re-administrations of a serotonergic psychedelic with the aim to increase the rate and tailor the reproducibility of peak experiences and to improve the therapeutic effect, reduce the side effects and improve the compliance rates may not be possible with other psychedelics, due to the late onset and long duration of psychedelic effects and due to the rapid development of tolerance (i.e. diminished or no psychedelic effects after re-administration) which can last for several days.

A patient as defined herein who suffers from negative thinking is treated by administration of 5-MeO-DMT or a pharmaceutically acceptable salt thereof.

In a preferred embodiment, the 5-MeO-DMT is administered as a monotherapy, i.e., the patient does not receive any other treatment for negative thinking.

The dosage amount of 5-MeO-DMT administered to a patient, as defined herein, suffering from negative thinking, is in the range of about 1 mg to about 25 mg, or any amount of range therein, preferably from about 2 mg to about 20 mg, more preferably from about 4 mg to about 20 mg. Useful specific amounts are e.g. about 4 mg, about 6 mg, about 8 mg, about 10 mg, about 12 mg, about 14 mg, about 16 mg, about 18 mg, and about 20 mg. Patients may also be treated with an equimolar dose of a pharmaceutically acceptable salt of 5-MeO-DMT, such as the hydrobromide salt. Note that in this specification, when ranges are set forth, such as “about 1 mg to about 25 mg,” the inventor contemplates all discrete values within that range, some of which are specifically mentioned, but all of which are not—simply for the purpose of brevity.

In preferred embodiments the improved methods for the treatment of a patient, as defined herein, suffering from negative thinking, with a therapeutically effective amount of 5-MeO-DMT, comprise the occurrence of a clinical response not later than about 2 hours after administration of 5-MeO-DMT.

In preferred embodiments the improved methods for the treatment of a patient, as defined herein, suffering from negative thinking, with a therapeutically effective amount of 5-MeO-DMT, comprise the persistence of a clinical response, including a clinical response which occurred not later than about 2 hours after administration of 5-MeO-DMT, until at least about 6 days after the last administration of 5-MeO-DMT, preferably until at least about 14 days after the last administration of 5-MeO-DMT, more preferably until at least about 28 days after the last administration of 5-MeO-DMT.

In preferred embodiments the improved methods for the treatment of a patient, as defined herein, suffering from negative thinking, with a therapeutically effective amount of 5-MeO-DMT comprise the administration of more than a single dose of 5-MeO-DMT.

In a preferred embodiment this more than a single dose of 5-MeO-DMT is administered to a patient in one or more treatment blocks, each block consisting of 2 to 7 administrations, with not less than about 1 hour and not more than about 24 hours between each administration within each treatment block, and not less than about 6 days between the end of one treatment block and the start of the next treatment block.

In an even more preferred embodiment this more than a single dose of 5-MeO-DMT is administered to a patient in one or more treatment blocks, each block consisting of 1 to 3 administrations, with about 24 hours between each administration within each treatment block, and not less than about 6 days between the end of one treatment block and the start of the next treatment block.

In a most preferred embodiment this more than a single dose of 5-MeO-DMT is administered to a patient in one or more treatment blocks, each block consisting of 1 to 3 administrations, with about 1 to 4 hours, preferably 1 to 2 hours, between each administration within each treatment block, and not less than about 6 days between the end of one treatment block and the start of the next treatment block.

In an embodiment the dosage amount of the 5-MeO-DMT administered to an individual patient in each of the administrations and in each of the treatment blocks is constant for that individual patient and is selected from about 1 mg to about 25 mg, preferably from about 2 mg to about 20 mg, more preferably from about 4 mg to about 20 mg. Useful specific amounts are e.g. about 4 mg, about 6 mg, about 8 mg, about 10 mg, about 12 mg, about 14 mg, about 16 mg, about 18 mg, and about 20 mg.

In a preferred embodiment the dosage amount of the 5-MeO-DMT administered to an individual patient is selected from about 2 mg to about 8 mg for the first administration within each treatment block, and then increases with each subsequent administration within each treatment block until the earlier of 20 mg being reached or all administrations within that treatment block being administered.

In an even more preferred embodiment the dosage amount of the 5-MeO-DMT administered to an individual patient is selected from about 2 mg to about 8 mg for the first administration within each treatment block, and then increases with each subsequent administration within each treatment block until the earlier of 20 mg being reached or all administrations within that treatment block being administered or the patient having experienced a peak psychedelic experience or the supervising physician having decided that further dose increases are inappropriate based on observed side effects.

For embodiments where the dosage amount increases for subsequent administrations, the dosage amount for the next administration is determined by adding about 2 mg to about 10 mg, preferably about 4 mg to about 8 mg, most preferably about 6 mg, to the dosage amount of the prior administration. For example, if the dosage amount of the first administration was 6 mg and the dosage amount increase is 6 mg, unless one of the previously mentioned stopping criteria has been reached, then the dosage amount of the second administration will be 12 mg. Preferably, the dosage amount for the third administration will be 18 mg.

In a preferred embodiment the dosage amount of the 5-MeO-DMT administered to an individual patient in each treatment block is selected from about 2 mg to about 8 mg for the first administration, and then increased, unless the patient has already experienced a peak psychedelic experience within that treatment block or the supervising physician has decided that further dose increases are inappropriate based on observed side effects, to a dosage selected from about 8 mg to about 14 mg for the second administration, and from about 14 mg to about 20 mg for the third administration. Useful specific amounts for the first, second and third administration are e.g. about 6 mg, about 12 mg, and about 18 mg.

In a further preferred embodiment the dosage amount of the 5-MeO-DMT administered to an individual patient is selected from about 2 mg to about 8 mg for the first administration of the first treatment block, and then increases with each subsequent administration within that first treatment block until the earlier of 20 mg being reached or all administrations within that treatment block being administered or the patient having experienced a peak psychedelic experience or the supervising physician having decided that further dose increases are inappropriate based on observed side effects, with that highest dosage in that first treatment block being used as the dosage for all subsequent treatment blocks and administrations within those subsequent treatment blocks. For example, if the highest dosage in the first treatment block was 18 mg because the patient experienced a peak psychedelic experience at that dose, then the dosage for all subsequent treatment blocks and administrations within those subsequent treatment blocks will be 18 mg.

In a most preferred embodiment the dosage amount of the 5-MeO-DMT administered to an individual patient is selected from about 2 mg to about 8 mg for the first administration of the first treatment block, and then increased, unless the patient has already experienced a peak psychedelic experience within that treatment block or the supervising physician has decided that further dose increases are inappropriate based on observed side effects, to a dosage selected from about 8 mg to about 14 mg for the second administration of the first treatment block, and from about 14 mg to about 20 mg for the third administration of the first treatment block, with the highest dosage in that first treatment block being used as the dosage for all subsequent treatment blocks and administrations within those subsequent treatment blocks. Useful specific amounts for the first, second and third administration in the first treatment block are e.g. about 6 mg, about 12 mg, and about 18 mg.

It is understood that a pharmaceutically acceptable salt of 5-MeO-DMT can also be used in all of the above dosing regimen, and that the appropriate weight amounts of a salt to be administered can be calculated from the stated weight amounts of the free base, assuming that equimolar amounts are used.

According to the invention, 5-MeO-DMT is preferably not administered together with a MAO inhibitor.

The occurrence of a “peak psychedelic experience” in a patient can be identified through achievement of at least 60% of the maximum possible score in each of the four subscales (mystical, positive mood, transcendence of time and space, and ineffability) of the 30-item revised Mystical Experience Questionnaire (MEQ-30) (as described in Barrett F S, J Psychopharmacol. 2015; 29(11):1182-90).

The occurrence of a “peak psychedelic experience” in a patient can also be identified through achievement of at least 60% of the maximum possible score of the Oceanic Boundlessness (OBN) dimension of the Altered States of Consciousness (ASC) questionnaire (as described in Roseman L et al., Front Pharmacol. 2018; 8:974).

In accordance with the invention, the occurrence of a “peak psychedelic experience” in a patient is preferably identified through achievement of a score of at least 75 in the Peak Experience Scale (PES) Total Score, also referred to as the Peak Psychedelic Experience Questionnaire (PPEQ), which averages answers scored by the patient from 0 to 100 for the following three questions: 1. How intense was the experience; 2. To what extent did you lose control; 3. How profound (i.e. deep and significant) was the experience?

### Treatment of Negative Thinking and Sleep Disturbance

Sleep disturbance refers to conditions, whether idiopathic or occurring in the context of a medical condition such as for example a mental disorder or a nervous system disorder, that affect sleep quality, timing, or duration. It impacts a person's ability to properly function while the person is awake and is linked to a pattern of high negative emotions and low positive emotions.

There are two fundamental types of sleep: rapid eye movement (REM) sleep and non-REM sleep. Non-REM sleep can be divided into four stages (I-IV). These non-REM stages correspond to an increasing depth of sleep. Non-REM and REM sleep alternate during each of the four to five cycles of normal human sleep each night. During the earlier proportion of the night, non-REM sleep is deeper and occupies a disproportionately large amount of time, particularly within the first cycle of sleep. As the night progresses, non-REM sleep becomes shallow and more of each cycle is allocated to REM sleep.

Normal healthy sleep consists of different phases as outlined above that proceed in successive, tightly regulated order through the night.

Disruption of this tight regulation results in sleep disturbances.

Common forms of sleep disturbances encompass disorders of initiating and maintaining sleep (insomnia), disorders of excessive somnolence (hypersomnia), disorders of sleep-wake schedule (circadian rhythm disorders), dysfunctions associated with sleep, sleep stages, or partial arousals (parasomnia), disorders characterized by respiratory disturbance during sleep (sleep-related breathing disorders) and disorders characterized by abnormal movements during sleep (sleep-related movement disorders). In a broad sense, fatigue can also be considered a sleep disturbance.

Insomnia is a sleep disturbance where people have difficulty falling or staying asleep. People with insomnia have difficulty falling asleep; wake up often during the night and have trouble going back to sleep; wake up too early in the morning; have unrefreshing sleep; and/or have at least one daytime problem such as fatigue, sleepiness, problems with mood, concentration, accidents at work or while driving, etc. due to poor sleep.

Hypersomnia is characterized by excessive daytime sleepiness, and/or prolonged nighttime sleep. Sleep drunkenness is also a symptom found in hypersomnia patients. It is a difficulty transitioning from sleep to wake. Individuals experiencing sleep drunkenness report waking with confusion, disorientation, slowness and repeated returns to sleep.

Circadian rhythm disorders are characterized by chronic or recurring sleep disturbances due to alterations of the individual's internal circadian rhythm or due to misalignments between their circadian rhythm and their desired or required work or social schedule. This dyssynchrony may be transient or persistent. The ensuing clinical picture combines elements of both insomnia and hypersomnia. Sleep periods are usually shortened and disrupted, performance during the desired waking state is impaired, and temporary opportunities to revert to a regular sleep schedule are unsuccessful.

Parasomnia designates various forms of sleep disturbance characterized by abnormal behavioural or physiological activity (such as sleepwalking or nightmares) that people experience prior to falling asleep, while asleep, or during the arousal period between sleep and wakefulness. There are considerable variations in terms of characteristics, severity, and frequency. Parasomnia may compromise the quality of sleep.

Sleep-related breathing disorders are characterized by abnormal and difficult respiration during sleep. Respiration is a complex process that relies heavily on the coordinated action of the muscles of respiration and the brain. With a potentially serious impact on sleep and the balance of oxygen and carbon dioxide in the blood, these sleep disturbances appear as chronic snoring, sleep apnoea, sleep related hypoventilation and/or hypoxemia. In some of these disorders, respiration is also abnormal during wakefulness. The reduction of airflow leads to intermittent hypoxia, with microarousals or awakenings, causing sleep fragmentation and excessive daytime sleepiness. Inflammation and endothelial dysfunction may ensue and reduce vascular elasticity and increase coagulation predisposing individuals to atherosclerosis, which, along with reduced oxygenation, may cause heart and brain damage.

In sleep-related movement disorders repetitive, relatively simple, usually stereotyped, movements interfere with sleep or its onset. The most common of these are restless leg syndrome (RLS) and periodic limb movement disorder (PLMD).

Fatigue describes a state of tiredness that does not resolve with rest or sleep. It is a feeling of exhaustion, lethargy, or decreased energy, usually experienced as a weakening or depletion of one's physical or mental resource and characterised by a decreased capacity for work and reduced efficiency in responding to stimuli. Fatigue is normal following a period of exertion, mental or physical, but sometimes may occur in the absence of such exertion as a symptom of health conditions.

Not getting the proper amount or quality of sleep may lead to personality changes and may not only exacerbate existing mental illness, but also be a trigger for the development of mental illness. Sleep disturbance may also lead to an impairment in emotional processing, which is able to perpetuate the opposite direction whereby daytime negative emotions and stress affect sleep quality and duration. Poor sleep quality is linked to a pattern of high negative emotions and low positive emotions. This is supported by the observation that individuals trained in experiential coping strategy for negative emotions were associated with increase sleep time and efficiency compared to controls. Further, guilt in the context of impulsivity was also found to be associated with sleep disturbance. Sleep loss can also adversely affect life by contributing to the development of obesity, diabetes and heart disease.

Treatment of sleep disturbance varies depending on the type and underlying cause. Maintenance of good sleep hygiene, a healthy sleep environment, and a consistent sleep-wake schedule are often considered as first-line treatment. If not successful, treatment also involves pharmacotherapy or psychotherapy.

Available treatments are not successful in all patients, may be associated with side effects and/or require treatment over a long period of time to achieve a relevant treatment effect.

In patients suffering from sleep disturbance in association with a mental disorder or a nervous system disorder known treatments of the mental or nervous system disorder do not necessarily improve the sleep disturbance.

For instance, sleep disturbance is frequently associated with mental disorders, such as depression. However, treatment of depression does not necessarily lead to an improvement of the concomitant sleep disturbances. While most antidepressants have been proven to influence the sleep architecture, some classes of antidepressants improve sleep, but others may cause sleep impairment (Hutka et al. Association of Sleep Architecture and Physiology with Depressive Disorder and Antidepressants Treatment. Int J Mol Sci. 2021 Jan. 29; 22(3):1333., Abstract).

To assess sleep, parameters such as sleep duration, sleep architecture, sleep latency, and the frequency and duration of awakenings throughout the night can be measured. The quantitative metrics may be measured using objective methods, including polysomnography, actigraphy, and the determination of sleep latency, or by way of self-reported measures (questionnaires).

Polysomnography is a technique requiring that a patient is monitored overnight at a specialized clinic. A variety of functions are measured throughout the night, including eye movements, brain and muscle activity, respiratory effort and airflow, blood oxygen levels, body positioning and movements, snoring, and heart rate.

Sleep latency can be measured by the multiple sleep latency test (MSLT). This test provides an objective measure to determine how long it takes a person to fall asleep across a multiplicity of test naps. An average sleep latency of approximately 10 minutes is considered to be normal; less than eight minutes is indicative of sleep disturbance. Accompanying analysis of brain activity can assist in the further diagnose of the sleep disturbance.

Sleep-rating questionnaires capture ratings of components of sleep quality, such as perceptions of sleep depth, rousing difficulties, and restfulness after sleep, in addition to other factors that could affect sleep quality, such as comorbid conditions and medication use.

The evaluation of the qualitative aspects of sleep experience is important, as sleep complaints can often persist despite normal values for quantitative measures of sleep.

Questionnaires not only facilitate a quick and accurate assessment of a complex clinical problem, but they are potentially also helpful for tracking a patient's progress. Thus, self-reported sleep questionnaires completed by patients are an important mainstay for the assessment of sleep disturbance in clinical practice.

Various sleep quality indexes are known. The following indexes include examples of questionnaires to assess sleep in general and questionnaires to assess in particular insomnia, hypersomnia, circadian rhythm disorders and parasomnia, respectively. The invention is, however, not limited to the use of a particular index or questionnaire.

Sleep quality in general can be assessed, for instance, with Sleep Quality Scale (SQS) and the Sleep-50 questionnaire.

The Sleep Quality Scale (SQS) is an all-inclusive assessment tool to achieve a general, efficient measure suitable for evaluating sleep quality in a variety of patient and research populations. Individual questions on daytime symptoms, such as attention, concentration difficulties or memory problems (Item 15, “Poor sleep makes hard for me to think”, item 19 “Poor sleep causes me to make mistakes at work”, item 21 “Poor sleep makes me forget things more easily”, item 22 “Poor sleep makes it hard to concentrate at work”), restoration after sleep, problems initiating and maintaining sleep, difficulty waking, and sleep satisfaction can be scored from 0 (“rarely”)-3 (“almost always”), with higher scores being indicative for more acute sleep problems.

The SLEEP-50 questionnaire consists of 50 items designed to screen for various kinds of sleep disturbance in the general population. The scale consists of nine subscales, reflecting some of the most common disorders and complaints related to sleep and the factors required for diagnosis such as sleep apnoea, insomnia, narcolepsy, restless legs/periodic leg movement disorder, circadian rhythm sleep disorder, sleepwalking, nightmares, factors influencing sleep, and the impact of sleep complaints on daily functioning. For each item, respondents are provided with a scale ranging from 1 (“not at all”) to 4 (“very much”) and are asked to indicate the extent to which the statement has matched their experience over the previous month or another appropriate recall window.

The questionnaire requires that for diagnosing a sleep disturbance not only the specific subscale (e.g., insomnia) exceeds a certain cut-off point, but also the impact subscale (Spoormaker et al. Initial validation of the SLEEP-50 questionnaire. Behav Sleep Med. 2005; 3(4):227-46., table 4 for optimal cut-off values and scoring procedures).

Treatment success is indicated (i) by a decrease of the score, preferably (ii) by a decrease to below the cut-off value.

A common questionnaire assessing sleep disturbance is the Pittsburgh Sleep Quality Index. Other instruments are the insomnia severity index and the Espie sleep disturbance questionnaire.

The Pittsburgh Sleep Quality Index (PSQI) assesses overall sleep quality and disturbances. The PSQI is a self-rated questionnaire comprising 19 questions. Respondents are asked to indicate how frequently they have experienced certain sleep difficulties over the past month or another appropriate recall window.

The 19 self-rated questions assess a wide variety of factors relating to sleep quality, including estimates of sleep duration and latency and of the frequency and severity of specific sleep-related problems. These 19 items are grouped into seven component scores: (1) subjective sleep quality; (2) sleep latency; (3) sleep duration; (4) habitual sleep efficiency; (5) sleep disturbances; (6) use of sleeping medication; (7) daytime dysfunction.

Each component is assigned a score of 0 to 3. Higher scores indicate more acute sleep disturbances. Detailed Scoring Instructions for the Pittsburgh Sleep Quality Index can be found in the Appendix of Buysse et al. The Pittsburgh Sleep Quality Index: a new instrument for psychiatric practice and research. Psychiatry Res. 1989 May; 28(2):193-213.

The seven component scores are then summed to yield one global score, with a range of 0-21 points, “0” indicating no difficulty and “21” indicating severe difficulties in all areas. A global score cut-off of 5 distinguishes poor from good sleepers. A global score >5 indicates that a patient is having severe difficulties in at least two areas, or moderate difficulties in more than three areas.

If treatment outcome is assessed using the PSQI, treatment success is indicated (i) by a decrease of the score, preferably (ii) by a decrease to 5 or below.

The Insomnia Severity Index (ISI) is a 5-item questionnaire relating to subjective sleep quality, severity of symptoms, subjective satisfaction with sleep, the degree to which insomnia interferes with daily functioning (Item 3, “To what extent do you consider your sleep problem to interfere with your daily functioning (e.g. daytime fatigue, ability to function at work/daily chores, concentration, memory, mood, etc).”), how noticeable the respondent feels his or her insomnia is compared to others, and the overall level of distress created by the sleep problem. Individual responses can be scored from 0 (=none) to 4 (=very); a higher total score corresponds to more severe insomnia. A total score of 0-7 indicates “no clinically significant insomnia,” 8-14 means “subthreshold insomnia,” 15-21 is “clinical insomnia (moderate severity),” and 22-28 means “clinical insomnia (severe)” (A. Shahid et al. (eds.), STOP, THAT and One Hundred Other Sleep Scales, Springer Science+Business Media, LLC 2012; Bastien et al. Validation of the Insomnia Severity Index as an outcome measure for insomnia research. Sleep Med. 2001 July; 2(4):297-307).

The usual recall window for the ISI is two weeks, but other appropriate recall windows can also be used herein.

Treatment success is indicated (i) by a decrease of the score, for instance, by >7 points, in particular >8 point; preferably (ii) by a decrease to below the cut-off value for clinically significant insomnia.

The Sleep Preoccupation Scale (SPS) is a 22-item, self-report scale, designed to assess daytime cognitions in patients with insomnia. Though researchers have frequently focused on nighttime thoughts and preoccupations when attempting to treat disordered sleep, a growing body of research suggests that daytime beliefs about sleep may be just as significant in the experience of insomnia. SPS items evaluate two distinct domains: the cognitive and behavioural consequences of poor sleep (e.g., negative thoughts and perceptions), and the affective consequences (e.g., worry and distress). The tool may be particularly useful for clinicians attempting to identify and treat the origins of sleep issues in their patients.

The Espie sleep disturbance questionnaire (SDQ) evaluates subjective experiences of insomnia. With ratings on restlessness/agitation, mental overactivity, consequences of insomnia, and lack of sleep readiness, the SDQ is concerned specifically with beliefs about the sources of sleep issues. Respondents use a five-point scale to indicate how often certain statements about insomnia are representative of their experience. 1 means “never true,” while 5 means “very often true.” Higher scores are indicative of more dysfunctional beliefs about the causes and correlates of insomnia (A. Shahid et al., loc. cit.; Espie et al. Insomniacs' attributions. psychometric properties of the Dysfunctional Beliefs and Attitudes about Sleep Scale and the Sleep Disturbance Questionnaire. J Psychosom Res. 2000 February; 48(2):141-8).

Treatment success is indicated by a decrease of the score.

Hypersomnia or hypersomnolence can be assessed by the Epworth sleepiness scale, the Stanford sleepiness scale, or the idiopathic hypersomnia severity scale.

The Epworth sleepiness scale (ESS) evaluates overall daytime sleepiness. The questionnaire asks respondents to rate how likely they are to fall asleep in eight different situations representing a moment of relative inactivity, such as a nap in the afternoon or sitting in a car stopped in traffic. Using a scale of 0-3 (with 0 meaning “would never doze” and 3 meaning “high chance of dozing”), respondents rate their likelihood of falling asleep. Scoring ranges from 0-24; the higher the score, the higher the severity of daytime sleepiness. A cut-off score of 10 identifies daytime sleepiness at a potentially clinical level (A. Shahid et al., loc. cit. Johns et al., A new method for measuring daytime sleepiness: the Epworth sleepiness scale. Sleep, 1991 December; 14(6):540-5).

Treatment success is indicated (i) by a decrease of the score, preferably (ii) by a decrease to 10 or below.

The Stanford sleepiness scale is a subjective measure of sleepiness, evaluating sleepiness at specific moments in time. Consisting of only one item, the scale requires respondents to select one of seven statements best representing their current level of perceived sleepiness. A scale from 1 (=Feeling active and vital; alert; wide awake) to 7 (=Almost in reverie; sleep onset soon; lost struggle to remain awake) is used to assess the level of sleepiness (A. Shahid et al., loc. cit.; Hoddes et al. The development and use of the Stanford sleepiness scale (SSS). Psychophysiology, 1972, 9, 150).

Treatment success is indicated by a decrease of the score.

Parasomnias can be evaluated by the Paris Arousal Disorders Severity Scale (PADSS).

The Paris Arousal Disorders Severity Scale (PADSS) is a self-rating scale listing parasomniac behaviours, assessing their frequency and includes an evaluation of consequences (Arnulf et al. A scale for assessing the severity of arousal disorders. Sleep. 2014 Jan. 1; 37(1):127-36).

Treatment success is indicated (i) by a decrease of the score, preferably (ii) by a decrease to below the cut-off value.

A common questionnaire that assesses sleep-related breathing disorders is the Berlin Questionnaire (A. Shahid et al., loc. cit.; Netzer et al., Using the Berlin Questionnaire to identify patients at risk for the sleep apnea syndrome. Ann Intern Med. 1999 Oct. 5; 131(7):485-91). An appropriate recall period can also be chosen.

Treatment success is indicated by a decrease of the score.

A common questionnaire that assesses sleep-related movement disorders is the International Restless Legs Syndrome Study Group Rating Scale (A. Shahid et al., loc. cit.; Walters et al.; International Restless Legs Syndrome Study Group 2003. Validation of the International Restless Legs Syndrome Study Group rating scale for restless legs syndrome. Sleep Medicine 4(2), pp. 121-132).

Treatment response can be assessed by a decrease of the score.

Fatigue is commonly assessed by for example the FACES (Fatigue, Anergy, Consciousness, Energy, Sleepiness).

The FACES (Fatigue, Anergy, Consciousness, Energy, Sleepiness) rating scale is a 50 items checklist to distinguish between tiredness, sleepiness, and fatigue. Respondents use a scale ranging from 0 (“not at all”) to 3 (“strongly”) to indicate the degree to which they have experienced each feeling or energy state over the course of the previous week or another appropriate recall period. Higher scores indicate more acute states of tiredness or fatigue, except for those items belonging to the energy subscale (A. Shahid et al., loc. cit.; Shapiro et al., Development of an adjective checklist to measure five FACES of fatigue and sleepiness. Data from a national survey of insomniacs. J Psychopomp Res. 2002 June; 52(6):467-73).

Effective treatment reduces scoring in tiredness and/or fatigue and/or increases scoring on the energy subscale.

Treatment response can be evaluated by above-mentioned quantitative measurements such as polysomnography or actigraphy and/or the use of the above-mentioned questionnaires. Depending on the respective sleep scale, a significant reduction or increment, respectively, in total score, or significant reduction of prevalence, frequency and impact on daily-functioning, respectively is indicative for treatment-induced improvement of the sleep disturbance.

Negative thinking in insomnia is assessed as part of sleep rating questionnaires as discussed above, for instance the Sleep Preoccupation Scale (SPS) or the Insomnia Severity Index (ISI).

Common tests that assess negative thinking or aspects thereof that can be used include the State Shame and Guilt Scale (SSGS) and the Positive and Negative Affect Schedule-Expanded Form (PANAS-X).

To enhance the understanding of the pathophysiology and potential compensatory mechanisms at play, resting-state fMRI has been widely applied in patients with sleep disturbances.

Altered resting state networks can be found in insomnia, hypersomnia, circadian rhythm disorders, parasomnias, sleep-related breathing disorders and sleep-related movement disorders.

In insomnia patients, dysfunctional connectivity is observed within the default mode network (DMN) and within the salience network, which is implicated in the detection and integration of emotional and sensory stimuli. Studies have suggested that these networks contain critical regions integrating emotional and bodily states, and the dysfunctional connectivity with other brain areas may underlie the vigilance, subjective distress, and poor sleep continuity of patients.

For instance, sleep deprivation in healthy subjects leads to functional connectivity alterations within and/or between the default mode network, dorsal attention network and salience network and these brain functional connectivity changes somewhat resemble the vulnerability patterns of patients with Alzheimer's disease.

In hypersomnia patients, the default mode network is affected. For instance, in idiopathic hypersomnia, distinct DMN hubs—the precuneus and medial prefrontal cortex—demonstrate significant changes, and functional connectivity in the DMN correlates with self-reported sleepiness severity.

A study investigating differences between night shift nurses and day work nurses revealed that dysrhythmia of circadian rhythms contributes to resting-state functional changes in the cerebellum, involved in sleep regulation, and cognitive functions such as responsiveness and alertness. Moreover, the functional connectivity of the DMN is fundamentally different in early and late circadian phenotypes. Like other forms of sleep disturbance, circadian rhythm disorders can lead to changes in brain functional connectivity. Changes in resting state brain functional connectivity have been reported in various diseases with circadian rhythm disorders.

While functional brain imaging is technically difficult to perform during a parasomnia event, differences in the precuneus have been observed in disorders of arousal representing a non-REM parasomnia.

The precuneus is involved in the analysis and integration of visual, audio, and somesthetic information and the monitoring of movements. The precuneus is a subregion of the DMN. Thus, in patients with parasomnias the default mode network is affected.

Resting-state fMRI studies in patients suffering from sleep-related breathing disorders, such as obstructive sleep apnoea (OSA), have demonstrated that long-term exposure to oxidative stress, intermittent hypoxia, hypo- and hypercapnia and sleep fragmentation, some of the major causes of OSA brain injury, may lead to significant global and regional connectivity deficits, especially in the default mode network (DMN) and regions involved in the arousal and sensorimotor systems.

Sleep-related movement disorders, such as for example periodic limb movements during sleep are reflected by alterations in the prefrontal motor control pathway, a subregion of the default mode network. Also, activity in the cerebellum and thalamus, with additional activation in the red nuclei and brainstem can be observed.

In many instances, resting state networks involved in sleep regulation are also involved in negative thinking. Thus, according to the invention, influencing those networks by a therapy according to the invention will lead to an improvement of the sleep disturbance and, if the patient treated suffers from negative thinking, also of the negative thinking.

Clinical data from studies of patients suffering from TRD or Postpartum Depression (PPD) confirm that sleep disturbance can successfully be treated.

In the studies, which involved the administration of 5-MeO-DMT, among others the MADRS item “reduced sleep”, which reflects insomnia, was assessed.

The MADRS item “reduced sleep” represents the experience of reduced duration or depth of sleep compared to the subject's own normal pattern when well. A score of 0 is assigned when the subject sleeps as usual. A score of 2 reflects slight difficulty dropping off to sleep or slightly reduced, light or fitful sleep. A score of 4 means that sleep is reduced or broken by at least two hours. A score of 6 means less than two or three hours sleep.

In the study group receiving the individualized dosing regimen, the aggregated score for the MADRS item “reduced sleep” across all 8 patients was 25 at base line. At day 1 after treatment, the earliest timepoint for assessing an impact of the treatment on sleep, it was reduced to 12 which corresponds to an improvement of 13 points or 52%. At day 7 after treatment, it was reduced to 9 which corresponds to an improvement of 16 points or 64%.

In the 12 mg group, the aggregated score for the MADRS item “reduced sleep” across all 4 patients was 12 at base line. At day 1 after treatment, it was reduced to 10 which corresponds to an improvement of 2 points or 17%. At day 7 after treatment, it was reduced to 6 which corresponds to an improvement of 6 points or 50%.

Thus, the score of the scale item that is of particular relevance to sleep disturbance, “reduced sleep”, is markedly improved. The inventors conclude that 5-MeO-DMT can be used to treat sleep disturbance, in particular patients suffering from a mental disorder or a nervous system disorder.

Treating a patient suffering from sleep disturbance, in particular insomnia, and associated negative thinking with 5-MeO-DMT or a pharmaceutically acceptable salt thereof reduces or eliminates the negative thinking and leads to an improvement of the sleep disturbance, in particular the insomnia.

The reduction or elimination of negative thinking in a patient suffering from sleep disturbance, in particular insomnia, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from sleep disturbance, in particular insomnia, occurs not later than about 2 hours after the last administration of 5-MeO-DMT or a pharmaceutically acceptable salt thereof. The reduction or elimination of negative thinking in a patient suffering from sleep disturbance, in particular insomnia, preferably persists until at least 6 days; in particular until at least 14 days; more preferably until at least 28 days after the last administration of 5-MeO-DMT or a pharmaceutically acceptable salt thereof.

The reduction or elimination of negative thinking, in particular of guilt, in a patient suffering from sleep disturbance, in particular insomnia, is reflected by at least an improvement in the score of the guilt subscale of the State Shame and Guilt Scale (SSGS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking, in particular of guilt, in a patient suffering from sleep disturbance, in particular insomnia, as reflected by at least an improvement in the score of the guilt subscale of the State Shame and Guilt Scale (SSGS), occurs not later than about 2 hours after the last administration of 5-MeO-DMT or a pharmaceutically acceptable salt thereof. The reduction or elimination of negative thinking, in particular of guilt, in a patient suffering from sleep disturbance, in particular insomnia, as reflected by at least an improvement in the score of the guilt subscale of the State Shame and Guilt Scale (SSGS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from sleep disturbance, in particular insomnia, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from sleep disturbance, in particular insomnia, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from sleep disturbance, in particular insomnia, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

As indicated above, negative thinking is an important aspect in patients suffering from a sleep disturbance. An improvement in negative thinking will therefore also lead to an improvement of the sleep disturbance. Since negative thinking furthermore also affects other aspects of the sleep disturbance, the inventors conclude that the improvement in negative thinking will additionally contribute to an overall improvement of the sleep disturbance, in particular insomnia.

In cases of idiopathic sleep disturbance in a patient also suffering from associated negative thinking, a clinical response may be reflected by a reduction in the Clinical Global Impression-Severity (CGI-S) score. According to the invention, a reduction in the CGI-S score means that the CGI-S is reduced by at least 1. Preferably, the CGI-S is reduced by at least 2 and/or to a score of 0. It is especially preferred if the CGI-S is reduced by at least 3 and/or to a score of 0.

An improvement of idiopathic sleep disturbance in a patient also suffering from associated negative thinking, as reflected by a reduction in the CGI-S score, is observed on day 1, for instance after about 24 hours; on day 7; on day 14; and/or on day 28 after the last administration of 5-MeO-DMT or a pharmaceutically acceptable salt thereof.

An improvement of idiopathic sleep disturbance in a patient also suffering from associated negative thinking, as reflected by a reduction in the CGI-S score in a patient also suffering from associated negative thinking, occurs not later than about 24 hours after the last administration of 5-MeO-DMT or a pharmaceutically acceptable salt thereof. An improvement in idiopathic sleep disturbance in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

In an embodiment, an improvement in idiopathic sleep disturbance in a patient also suffering from associated negative thinking, as reflected by a reduction in the CGI-S score, is observed on day 7 after the last administration of 5-MeO-DMT or a pharmaceutically acceptable salt thereof and preferably persists until at least 14 days, more preferably until at least 28 days.

In cases of idiopathic sleep disturbance in a patient also suffering from associated negative thinking, an improvement in sleep disturbance, as reflected by at least a score of “much improved” in the Clinical Global Impression-Improvement (CGI-I) score or the Patient Global Impression-Improvement (PGI-I) score, preferably occurs not later than about 24 hours after the last administration of 5-MeO-DMT or a pharmaceutically acceptable salt thereof.

In cases of idiopathic sleep disturbance in a patient also suffering from associated negative thinking, an improvement in sleep disturbance, as reflected by a reduction by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

Improvements in cases of idiopathic sleep disturbance in a patient also suffering from associated negative thinking may also be assessed by any other scale reflecting changes in sleep quality or quantity, as indicated above, for instance the Pittsburgh Sleep Quality Index (PSOI).

If treatment outcome is assessed using the PSQI, treatment success is indicated (i) by a decrease of the score, preferably (ii) by a decrease to 5 or below.

An improvement in idiopathic sleep disturbance in a patient also suffering from associated negative thinking, as reflected by a decrease of the PSQI global score, in particular by a decrease to 5 or below, preferably occurs not later than about 24 hours after the last administration of 5-MeO-DMT or a pharmaceutically acceptable salt thereof, wherein the recall period spans from the time point when acute psychedelic experiences have subsided after the last administration to the assessment time point.

Such an improvement in idiopathic sleep disturbance in a patient also suffering from associated negative thinking, as reflected by a decrease of the PSQI global score, preferably persists until at least 6 days; in particular until at least 14 days; more preferably until at least 28 days after the last administration of 5-MeO-DMT or a pharmaceutically acceptable salt thereof, wherein the recall period spans from the time point when acute psychedelic experiences have subsided after the last administration to the assessment time point.

An improvement of idiopathic sleep disturbance in a patient also suffering from associated negative thinking, as reflected by a reduction in the PSQI global score, is observed on day 1, for instance after about 24 hours; on day 7; on day 14; and/or on day 28 after the last administration of 5-MeO-DMT or a pharmaceutically acceptable salt thereof, wherein the recall period spans from the time point when acute psychedelic experiences have subsided after the last administration to the assessment time point.

Negative thinking and sleep disturbance, in particular insomnia, are closely linked to mental and nervous system disorders. Both, negative thinking and sleep disturbance, in particular insomnia, occur in mental or nervous system disorders, such as a disorder characterized by depressive episodes, for example, Major Depressive Disorder (MDD), Bipolar Disorder (BD), such as Bipolar I Disorder and Bipolar II Disorder, Postpartum Depression (PPD), Seasonal Affective Disorder and Persistent Depressive Disorder; Anxiety Disorder, for example Generalised Anxiety Disorder (GAD) and Social Anxiety Disorder (SAD); Obsessive Compulsive and Related Disorders, for example, Obsessive Compulsive Disorder (OCD) and Body Dysmorphic Disorder (BDD); Post-Traumatic Stress Disorder (PTSD); Pain Disorders, for example, Chronic Pain; Mental and Behavioural Disorders due to Psychoactive Substance Use, for example Substance Use Disorder (SUD); Psychotic Disorder, for example Schizophrenia; Dementia, for example Alzheimer's Dementia (AD); Eating Disorders; Attention Deficit Hyperactivity Disorder (ADHD); Personality Disorders, for example Schizotypal Personality Disorder and Borderline Personality Disorder (BPD).

Negative thinking or and sleep disturbance, in particular insomnia, also both occur in medical health conditions leading to an associated mental or nervous system condition, such as Traumatic Brain Injury (TBI).

A treatment according to the invention will lead to improvements in both negative thinking and sleep disturbance, in particular insomnia, and furthermore to an improvement in the associated mental or nervous system disorder, examples of which are listed above.

### Treatment of Negative Thinking and Mental and Nervous System Disorders

In patients suffering from negative thinking in association with a mental disorder or a nervous system disorder a treatment of negative thinking according to the invention leads to an improvement of the condition with which the negative thinking is associated.

While negative thinking may be considered a condition deserving treatment independent of any other conditions, disorders or symptoms an individual may suffer from, several mental disorders and disorders of the nervous system are associated with negative thinking. Notably, the relationship between negative thinking and mental disorder is bidirectional. Not only can mental disorders have a negative impact on the emotional state of the patient, but negative thinking can also be a contributing factor to the onset, progression and prognosis of mental or nervous system disorders.

In many instances, resting state networks involved in negative thinking are also involved in the conditions listed above.

5-MeO-DMT has the ability to disrupt established functional connectivity patterns of resting state networks. This disruption leads to a reset of the pathological ill-connected brain connections as the networks reconnect. New, healthy functional connections are established with persistent effects.

### Disorders Characterized by Depressive Episodes

There are several disorders which are characterized by depressive episodes.

A depressive episode is a period of depressed mood and/or loss of pleasure in most activities.

For instance, according to DSM-V, a Major Depressive Episode is characterized by five or more symptoms that have been present during the same 2-week period and represent a change from previous functioning; at least one of the symptoms being either (1) depressed mood or (2) loss of interest or pleasure, including criteria such as feelings of hopelessness, worthlessness and excessive or inappropriate guilt.

The patient suffering from a disorder characterized by depressive episodes may suffer from a treatment resistant form of the disorder.

Negative thinking occurs in patients suffering from disorders characterized by depressive episodes. It involves impairments in the regulation of emotions.

Negative thinking is included as a diagnostic criterion for disorders characterized by depressive episodes. It may be assessed as part of the determination of the HAM-D, the MADRS or the BPRS.

The Montgomery-Åsberg Depression Rating Scale (MADRS) is a ten-item diagnostic questionnaire used to measure the severity of depressive episodes in patients with mood disorders. Higher MADRS scores indicate more severe depression.

MADRS items are apparent sadness; reported sadness; inner tension; reduced sleep; reduced appetite; concentration difficulties; lassitude; inability to feel; pessimistic thoughts; and suicidal thoughts and each item yields a score of 0 to 6. The overall score ranges from 0 to 60.

Pessimistic thoughts, represents thoughts of guilt, inferiority, self-reproach, sinfulness, remorse and ruin are rated from “No pessimistic thoughts” (=0), “Fluctuating ideas of failure, self-reproach or self-depreciation.” (=1, 2 or 3), “Persistent self-accusations, or definite but still rational ideas of guilt or sin. Increasingly pessimistic about the future.” (=3, 4 or 5) to “Delusions of ruin, remorse or unredeemable sin. Self-accusations which are absurd and unshakable” (=6).

The “guilt feelings” item of the Brief Psychiatric Rating Scale (BPRS) represents over-concern or remorse for past behaviour. It is rated on the basis of the patient's subjective experiences of guilt as evidenced by verbal report with appropriate affect; guilt feelings from depression, anxiety or neurotic defences may not be inferred.

The Hamilton Rating Scale for Depression (HAM-D) allows clinicians to assess the nature and severity of mood disorders in patient populations. The scale is comprised of 21 items for inquiry, though only the first 17 (depressed mood; feelings of guilt; suicide; initial insomnia; insomnia during the night; delayed insomnia; work and interests; retardation; agitation; psychiatric anxiety; somatic anxiety; gastrointestinal somatic symptoms; general somatic symptoms; genital symptoms; hypochondriasis; weight loss; insight) are used in scoring.

For the majority of questions, scores range from 0 to 4, with 4 representing more acute signs of depression. Several questions have ranges that extend only as high as 2. A total score is tallied and can then be compared with previous scores or can be contrasted with a pre-defined cut-off score.

In the context of the item “depressed mood”, the questionnaire assesses sadness, hopeless, helpless, worthless. A further item assesses “feelings of guilt”.

Negative thinking in a patient suffering from a disorder characterized by depressive episodes can, for instance, be assessed using the State Shame and Guilt Scale (SSGS), the Positive and Negative Affect Schedule-Expanded Form (PANAS-X) or the State Hope Scale (SHS).

In patients suffering from a disorder characterized by depressive episodes altered functional connectivity is observed within and/or between several brain regions implicated in processing, regulation, emotional memory; cognitive processes related to rumination; impaired concentration and physiological arousal.

Treating a patient suffering from a disorder characterized by depressive episodes, including a treatment resistant form of the disorder, and associated negative thinking with 5-MeO-DMT or a pharmaceutically acceptable salt thereof reduces or eliminates the negative thinking and leads to an improvement of the disorder characterized by depressive episodes.

The reduction or elimination of negative thinking in a patient suffering from a disorder characterized by depressive episodes is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from a disorder characterized by depressive episodes occurs not later than about 2 hours after the last administration of 5-MeO-DMT or a pharmaceutically acceptable salt thereof. The reduction or elimination of negative thinking in a patient suffering from a disorder characterized by depressive episodes preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from a disorder characterized by depressive episodes, in particular of pessimistic thoughts, is reflected by an improvement at least in the score of the MADRS item “pessimistic thoughts” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from a disorder characterized by depressive episodes, in particular of pessimistic thoughts, as reflected by an improvement in the score of the MADRS item “pessimistic thoughts”, occurs not later than about 2 hours after the last administration of 5-MeO-DMT or a pharmaceutically acceptable salt thereof. The reduction or elimination of negative thinking in a patient suffering from a disorder characterized by depressive episodes, as reflected by an improvement in the score of the MADRS item “pessimistic thoughts”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from a disorder characterized by depressive episodes, in particular of guilt feelings, is reflected by an improvement at least in the score of the BPRS item “guilt feelings” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from a disorder characterized by depressive episodes, in particular of guilt feelings, as reflected by an improvement in the score of the BPRS item “guilt feelings”, occurs not later than about 2 hours after the last administration of 5-MeO-DMT or a pharmaceutically acceptable salt thereof. The reduction or elimination of negative thinking in a patient suffering from a disorder characterized by depressive episodes, as reflected by an improvement in the score of the BPRS item “guilt feelings”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from a disorder characterized by depressive episodes, in particular of depressed mood, is reflected by at least an improvement in the score of the HAM-D item “depressed mood” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from a disorder characterized by depressive episodes, in particular of depressed mood, as reflected by an improvement in the score of the HAM-D item “depressed mood”, occurs not later than about 2 hours after the last administration of 5-MeO-DMT or a pharmaceutically acceptable salt thereof. The reduction or elimination of negative thinking in a patient suffering from a disorder characterized by depressive episodes, in particular of depressed mood, as reflected by an improvement in the score of the HAM-D item “depressed mood”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from a disorder characterized by depressive episodes, in particular of feelings of guilt, is reflected by at least an improvement in the score of the HAM-D item “feelings of guilt” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from a disorder characterized by depressive episodes, in particular of feelings of guilt, as reflected by an improvement in the score of the HAM-D item “feelings of guilt”, occurs not later than about 2 hours after the last administration of 5-MeO-DMT or a pharmaceutically acceptable salt thereof. The reduction or elimination of negative thinking in a patient suffering from a disorder characterized by depressive episodes, in particular of feelings of guilt, as reflected by an improvement in the score of the HAM-D item “feelings of guilt”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from a disorder characterized by depressive episodes, is reflected by at least an improvement in the score of the guilt subscale of the State Shame and Guilt Scale (SSGS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from a disorder characterized by depressive episodes, as reflected by at least an improvement in the score of the guilt subscale of the State Shame and Guilt Scale (SSGS), occurs not later than about 2 hours after the last administration of 5-MeO-DMT or a pharmaceutically acceptable salt thereof. The reduction or elimination of negative thinking in a patient suffering from a disorder characterized by depressive episodes, as reflected by at least an improvement in the score of the guilt subscale of the State Shame and Guilt Scale (SSGS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from a disorder characterized by depressive episodes, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from a disorder characterized by depressive episodes, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking preferably in a patient suffering from a disorder characterized by depressive episodes, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

Alternatively or additionally, the reduction or elimination of negative thinking in a patient suffering from a disorder characterized by depressive episodes, in particular of hopelessness, is reflected by at least an improvement in the score of the State Hope Scale (SHS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from a disorder characterized by depressive episodes, in particular of hopelessness, as reflected by at least an improvement in the score of the State Hope Scale (SHS), occurs not later than about 2 hours after the last administration of 5-MeO-DMT or a pharmaceutically acceptable salt thereof. The reduction or elimination of negative thinking in a patient suffering from a disorder characterized by depressive episodes, in particular of hopelessness, as reflected by at least an improvement in the score of the State Hope Scale (SHS), preferably persists until at least 6 days; in particular until at least 14 days; more preferably until at least 28 days after the last administration of 5-MeO-DMT or a pharmaceutically acceptable salt thereof.

As indicated above, negative thinking is closely linked to disorder characterized by depressive episodes. An improvement in negative thinking will therefore also lead to an improvement of the disorder characterized by depressive episodes. Since negative thinking furthermore also affects other aspects of a disorder characterized by depressive episodes, the inventors conclude that the improvement in negative thinking will additionally contribute to an overall improvement of the disorder characterized by depressive episodes.

An improvement of the disorder characterized by depressive episodes in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the disorder characterized by depressive episodes in a patient also suffering from associated negative thinking, as reflected by a reduction in the CGI-S score, occurs not later than about 2 hours after the last administration of 5-MeO-DMT or a pharmaceutically acceptable salt thereof. An improvement of the disorder characterized by depressive episodes in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Major Depressive Disorder (MDD) is a mood disorder that causes a persistent feeling of sadness and loss of interest. It affects how a person feels, thinks and behaves and can lead to a variety of emotional and physical problems.

The patient suffering from MDD may suffer from a treatment resistant form of the disorder.

The patient may suffer from treatment resistant disease. Treatment resistance means that the patient had no adequate improvement after at least two adequate courses of therapy. The patient in particular had no adequate improvement after at least two adequate courses of therapy, wherein at least one of the two courses was a pharmacotherapy; for instance, the patient had no adequate improvement after at least two adequate courses of pharmacotherapy. The at least two prior courses of treatment were in particular administered in the current episode of depression.

Negative feelings such as guilt, hopelessness and worthlessness are core features of MDD. They are included in the list of 9 features of which 5 are needed for at least 2 weeks to qualify for a DSM diagnosis of Major Depressive Disorder.

Guilt has been found to be a common feature in a number of different cultures, with some variation in prevalence between different countries.

Negative thinking in a patient suffering from MDD may be assessed as part of the determination of the HAM-D, the MADRS or the BPRS. Negative thinking or at least aspects thereof may furthermore be assessed, for instance, by using the State Shame and Guilt Scale (SSGS), the Positive and Negative Affect Schedule-Expanded Form (PANAS-X) or the State Hope Scale (SHS).

In patients suffering from MDD dysfunctional connectivity and regulation within and/or between multiple resting-state networks including the DMN, salience network, executive control network and limbic network is observed. Functional connectivity differs significantly from that observed in healthy controls.

Treating a patient suffering from MDD, including a treatment resistant form of the disorder, and associated negative thinking with 5-MeO-DMT or a pharmaceutically acceptable salt thereof reduces or eliminates the negative thinking and leads to an improvement of MDD.

The patient may suffer from moderate or severe MDD as indicated by a Montgomery-Åsberg Depression Rating Scale (MADRS) score of 20 or more or by a Hamilton Depression Rating Scale (HAM-D) score of 17 or more. It is further considered that the patient may suffers from severe major depressive disorder as indicated by a Montgomery-Åsberg Depression Rating Scale (MADRS) score of 35 or more or by a Hamilton Depression Rating Scale (HAM-D) score of 25 or more.

The reduction or elimination of negative thinking in a patient suffering from MDD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from MDD occurs not later than about 2 hours after the last administration of 5-MeO-DMT or a pharmaceutically acceptable salt thereof. The reduction or elimination of negative thinking in a patient suffering from MDD preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from MDD, in particular of pessimistic thoughts, is reflected by an improvement at least in the score of the MADRS item “pessimistic thoughts” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from MDD, in particular of pessimistic thoughts, as reflected by an improvement in the score of the MADRS item “pessimistic thoughts” occurs not later than about 2 hours after the last administration of 5-MeO-DMT or a pharmaceutically acceptable salt thereof. The reduction or elimination of negative thinking in a patient suffering from MDD, in particular of pessimistic thoughts, as reflected by an improvement in the score of the MADRS item “pessimistic thoughts”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from MDD, in particular of guilt feelings, is reflected by at least an improvement in the score of the BPRS item “guilt feelings” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from MDD, in particular of guilt feelings, as reflected by an improvement in the score of the BPRS item “guilt feelings”, occurs not later than about 2 hours after the last administration of 5-MeO-DMT or a pharmaceutically acceptable salt thereof. The reduction or elimination of negative thinking in a patient suffering from MDD, in particular of guilt feelings, as reflected by an improvement in the score of the BPRS item “guilt feelings”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from MDD, in particular of depressed mood, is reflected by at least an improvement in the score of the HAM-D item “depressed mood” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from MDD, in particular of depressed mood, as reflected by an improvement in the score of the HAM-D item “depressed mood”, occurs not later than about 2 hours after the last administration of 5-MeO-DMT or a pharmaceutically acceptable salt thereof. The reduction or elimination of negative thinking in a patient suffering from MDD, in particular of depressed mood, as reflected by an improvement in the score of the HAM-D item “depressed mood”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from MDD, in particular of feelings of guilt, is reflected by at least an improvement in the score of the HAM-D item “feelings of guilt” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from MDD, in particular of feelings of guilt, as reflected by an improvement in the score of the HAM-D item “feelings of guilt”, occurs not later than about 2 hours after the last administration of 5-MeO-DMT or a pharmaceutically acceptable salt thereof. The reduction or elimination of negative thinking in a patient suffering from MDD, in particular of feelings of guilt, as reflected by an improvement in the score of the HAM-D item “feelings of guilt”, preferably persists until at least 6 days; in particular until at least 14 days; more preferably until at least 28 days after the last administration of 5-MeO-DMT or a pharmaceutically acceptable salt thereof.

Alternatively or additionally, a reduction or elimination of negative thinking in a patient suffering from MDD, is reflected by at least an improvement in the score of the guilt subscale of the State Shame and Guilt Scale (SSGS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of negative thinking in a patient suffering MDD, as reflected by at least an improvement in the score of the guilt subscale of the State Shame and Guilt Scale (SSGS), occurs not later than about 2 hours after the last administration of 5-MeO-DMT or a pharmaceutically acceptable salt thereof. The reduction or elimination of negative thinking n a patient suffering MDD, as reflected by at least an improvement in the score of the guilt subscale of the State Shame and Guilt Scale (SSGS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from MDD, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from MDD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from MDD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

Alternatively or additionally, the reduction or elimination of negative thinking in a patient suffering from MDD, in particular of hopelessness, is reflected by at least an improvement in the score of the State Hope Scale (SHS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from MDD, in particular of hopelessness, as reflected by at least an improvement in the score of the State Hope Scale (SHS), occurs not later than about 2 hours after the last administration of 5-MeO-DMT or a pharmaceutically acceptable salt thereof. The reduction or elimination of negative thinking in a patient suffering from MDD, in particular of hopelessness, as reflected by at least an improvement in the score of the State Hope Scale (SHS), preferably persists until at least 6 days; in particular until at least 14 days; more preferably until at least 28 days after the last administration of 5-MeO-DMT or a pharmaceutically acceptable salt thereof.

As indicated above, negative thinking is closely linked to MDD. An improvement in negative thinking will therefore also lead to an improvement of MDD. Since negative thinking furthermore also affects other aspects of MDD, the inventors conclude that the improvement in negative thinking will additionally contribute to an overall improvement of MDD.

An improvement of MDD in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of MDD in a patient also suffering from associated negative thinking, as reflected by a reduction in the CGI-S score, occurs not later than about 2 hours after the last administration of 5-MeO-DMT or a pharmaceutically acceptable salt thereof. An improvement of MDD in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Compared to other psychedelics, like LSD, psylocibin or DMT, 5-MeO-DMT or a pharmaceutical acceptable salt thereof can be administered to patients suffering from MDD associated with negative thinking, preferably using dosing schemes as described herein, without a significant risk of inducing mania or hypomania.

Preferably, the patient suffering from MDD associated with negative thinking does not experience treatment-emergent mania or hypomania.

Bipolar Disorder (BD) is a mental health condition characterized by extreme mood swings that include emotional lows (major depressive episodes) and highs (manic or hypomanic episodes). BD is a recurrent chronic disorder that affects more than 1% of the world's population irrespective of ethnic origin or socioeconomic status.

BD is classified as Bipolar I Disorder if there has been at least one manic episode, with or without depressive episodes. It is classified as Bipolar II Disorder if there has been at least one hypomanic episode (but no full manic episodes) and one major depressive episode. If these symptoms are due to drugs or medical problems, they are not diagnosed as Bipolar Disorder.

The patient suffering from BD including Bipolar I Disorder and Bipolar II Disorder may suffer from a treatment resistant form of the disorder.

The patient may suffer from treatment resistant disease. Treatment resistance means that the patient had no adequate improvement after at least two adequate courses of therapy. The patient in particular had no adequate improvement after at least two adequate courses of therapy, wherein at least one of the two courses was a pharmacotherapy; for instance, the patient had no adequate improvement after at least two adequate courses of pharmacotherapy. The at least two prior courses of treatment were in particular administered in the current episode of depression.

A study comparing 67 patients with bipolar I disorder to well-matched controls found that bipolar was linked to a number of emotional disturbances, but in particular was strongly linked to elevations in negative emotions.

Distinct brain regions of patients with bipolar disorder have been implicated in mood symptoms and feelings of guilt.

When comparing features of bipolar depression to features of unipolar depression, pathological guilt was found to be a significant feature over-represented in bipolar depression.

Negative feelings such as guilt, hopelessness and worthlessness are also core features of BD, which can be characterised by a major depressive episode. They are included in the list of 9 features of which 5 are needed for at least 2 weeks to qualify for a DSM diagnosis of BD.

Negative thinking in a patient suffering from BD may be assessed as part of the determination of the HAM-D, the MADRS, the BPRS or the BDRS. Negative thinking or at least aspects thereof may furthermore be assessed, for instance, by using the State Shame and Guilt Scale (SSGS), the Positive and Negative Affect Schedule-Expanded Form (PANAS-X) or the State Hope Scale (SHS).

The Bipolar Depression Rating Scale (BDRS) is designed to measure the severity of depressive symptoms in bipolar depression. The BDRS is validated for clinical use by trained raters. Based on a clinical interview, the BDRS items rate the severity of depressive and/or mixed symptoms expressed by patients currently and during the past few days. If there is a discordance between symptoms currently and the last few days, the rating should reflect current symptoms. The scale contains 20 questions and the maximum score possible is 60. Higher scores indicate greater severity.

The questions address depressed mood; sleep disturbance; appetite disturbance; reduced social engagement; reduced energy and activity; reduced motivation; impaired concentration and memory; anxiety; anhedonia; affective flattening; feelings of worthlessness; feelings of helplessness and hopelessness; suicidal ideation; feelings of guilt; psychotic symptoms; irritability; lability; increased motor drive; increased speech; agitation.

Each of these aspects is assessed and assigned a score of 0, 1, 2 or 3.

The BDRS item worthlessness is scored as 0 if there is no subjective sense, or thoughts, of decreased self-value or self-worth; 1 (mild) in the case of slight decrease in sense of self-worth; 2 (moderate) in the case of some thoughts of worthlessness and decreased self-worth; 3 (severe) in the case of marked, pervasive, or persistent feelings of worthlessness, e.g., feels others better off without them, unable to appreciate positive attributes.

The BDRS item helplessness and hopelessness is scored with 0 if there is no subjective sense of pessimism or gloom regarding the future, inability to cope, or sense of loss of control; 1 (mild) in the case of occasional and mild feelings of not being able to cope as usual; or pessimism; 2 (moderate) in the case of often feels unable to cope, or significant feelings of helplessness or hopelessness which lift at times; 3 (severe) in the case of marked and persistent feelings of pessimism, helplessness, or hopelessness.

The BDRS item guilt is scored with 0 if there is no subjective sense of self blame, failure, or remorse for real or imagined past errors; 1 (mild) in the case of slight decrease in self-esteem or increased self-criticism; 2 (moderate) in the case of significant thoughts of failure, self-criticism, inability to cope, or ruminations regarding past failures and the effect on others; able to recognise as excessive; 3 (severe) in the case of marked, pervasive, or persistent guilt, e.g., feelings of deserving punishment; or does not clearly recognise as excessive.

Patients suffering from Bipolar Disorder show characteristic aberrant intrinsic organization and interconnectivity of resting state networks. In comparison with healthy controls, resting state functional magnetic resonance imaging studies demonstrated functional connectivity alterations of specific regions within and/or between the default mode network, the salience network, and the central executive network. In particular functional connectivity alterations within the default mode network are also observed in patients suffering from sleep disturbance.

Treating a patient suffering from BD, including a treatment resistant form of the disorder, and associated negative thinking with 5-MeO-DMT or a pharmaceutically acceptable salt thereof reduces or eliminates the negative thinking and leads to an improvement of BD.

Typically, the patient suffering from BD, whether diagnosed with bipolar II disorder or with bipolar I disorder, suffers from a current major depressive episode.

The severity of a current major depressive episode may be assessed using the Montgomery-Åsberg Depression Rating Scale (MADRS). The patient may have a total score of equal to or greater than 19, such as greater or equal than 24, in particular greater or equal than 37.

Alternatively or in addition, the patient may have a Bipolar Depression Rating Scale (BDRS) total score of 19, such as greater or equal than 24, in particular greater or equal than 37.

The reduction or elimination of negative thinking in a patient suffering from BD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from BD occurs not later than about 2 hours after the last administration of 5-MeO-DMT or a pharmaceutically acceptable salt thereof. The reduction or elimination of negative thinking in a patient suffering from BD preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from BD, in particular of pessimistic thoughts, is reflected by an improvement at least in the score of the MADRS item “pessimistic thoughts” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from BD, in particular of pessimistic thoughts, as reflected by an improvement in the score of the MADRS item “pessimistic thoughts” occurs not later than about 2 hours after the last administration of 5-MeO-DMT or a pharmaceutically acceptable salt thereof. The reduction or elimination of negative thinking in a patient suffering from BD, in particular of pessimistic thoughts, as reflected by an improvement in the score of the MADRS item “pessimistic thoughts” preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from BD, in particular of guilt feelings, is reflected by an improvement at least in the score of the BPRS item “guilt feelings” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from BD, in particular of guilt feelings, as reflected by an improvement in the score of the BPRS item “guilt feelings” occurs not later than about 2 hours after the last administration of 5-MeO-DMT or a pharmaceutically acceptable salt thereof. The reduction or elimination of negative thinking in a patient suffering from BD, in particular of guilt feelings, as reflected by an improvement in the score of the BPRS item “guilt feelings” preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from BD, in particular of depressed mood, is reflected by at least an improvement in the score of the HAM-D item “depressed mood” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from BD, in particular of depressed mood, as reflected by an improvement in the score of the HAM-D item “depressed mood”, occurs not later than about 2 hours after the last administration of 5-MeO-DMT or a pharmaceutically acceptable salt thereof. The reduction or elimination of negative thinking in a patient suffering from BD, in particular of depressed mood, as reflected by an improvement in the score of the HAM-D item “depressed mood”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from BD, in particular of feelings of guilt, is reflected by at least an improvement in the score of the HAM-D item “feelings of guilt” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from BD, in particular of feelings of guilt, as reflected by an improvement in the score of the HAM-D item “feelings of guilt”, occurs not later than about 2 hours after the last administration of 5-MeO-DMT or a pharmaceutically acceptable salt thereof. The reduction or elimination of negative thinking in a patient suffering from BD, in particular of feelings of guilt, as reflected by an improvement in the score of the HAM-D item “feelings of guilt”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from BD, in particular of worthlessness, is reflected by at least an improvement in the score of the BDRS item worthlessness about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from BD, in particular of worthlessness, as reflected by an improvement in the score of the BDRS item worthlessness, occurs not later than about 2 hours after the last administration of 5-MeO-DMT or a pharmaceutically acceptable salt thereof. The reduction or elimination of negative thinking in a patient suffering from BD, in particular of worthlessness, as reflected by an improvement in the score of the BDRS item worthlessness, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from BD, in particular of helplessness and hopelessness, is reflected by at least an improvement in the score of the BDRS item helplessness and hopelessness about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from BD, in particular of helplessness and hopelessness, as reflected by an improvement in the score of the BDRS item helplessness and hopelessness, occurs not later than about 2 hours after the last administration of 5-MeO-DMT or a pharmaceutically acceptable salt thereof. The reduction or elimination of negative thinking in a patient suffering from BD, in particular of helplessness and hopelessness, as reflected by an improvement in the score of the BDRS item helplessness and hopelessness, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from BD, in particular of guilt, is reflected by at least an improvement in the score of the BDRS item guilt about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from BD, in particular of guilt, as reflected by an improvement in the score of the BDRS item guilt, occurs not later than about 2 hours after the last administration of 5-MeO-DMT or a pharmaceutically acceptable salt thereof. The reduction or elimination of negative thinking in a patient suffering from BD, in particular of guilt, as reflected by an improvement in the score of the BDRS item guilt, preferably persists until at least 6 days; in particular until at least 14 days; more preferably until at least 28 days after the last administration of 5-MeO-DMT or a pharmaceutically acceptable salt thereof.

Alternatively or additionally, a reduction or elimination of negative thinking in a patient suffering from BD, is reflected by at least an improvement in the score of the guilt subscale of the State Shame and Guilt Scale (SSGS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of negative thinking in a patient suffering BD, as reflected by at least an improvement in the score of the guilt subscale of the State Shame and Guilt Scale (SSGS), occurs not later than about 2 hours after the last administration of 5-MeO-DMT or a pharmaceutically acceptable salt thereof. The reduction or elimination of negative thinking in a patient suffering BD, as reflected by at least an improvement in the score of the guilt subscale of the State Shame and Guilt Scale (SSGS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from BD, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from BD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from BD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

Alternatively or additionally, the reduction or elimination of negative thinking in a patient suffering from BD, in particular of hopelessness, is reflected by at least an improvement in the score of the State Hope Scale (SHS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from BD, in particular of hopelessness, as reflected by at least an improvement in the score of the State Hope Scale (SHS), occurs not later than about 2 hours after the last administration of 5-MeO-DMT or a pharmaceutically acceptable salt thereof. The reduction or elimination of negative thinking in a patient suffering from BD, as reflected by at least an improvement in the score of the State Hope Scale (SHS), preferably persists until at least 6 days; in particular until at least 14 days; more preferably until at least 28 days after the last administration of 5-MeO-DMT or a pharmaceutically acceptable salt thereof.

As indicated above, negative thinking is closely linked to BD. An improvement in negative thinking will therefore also lead to an improvement of BD. Since negative thinking furthermore also affects other aspects of BD, the inventors conclude that the improvement in negative thinking will additionally contribute to an overall improvement of BD.

An improvement of BD in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of BD in a patient also suffering from associated negative thinking, as reflected by a reduction in the CGI-S score, occurs not later than about 2 hours after the last administration of 5-MeO-DMT or a pharmaceutically acceptable salt thereof. An improvement of BD in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Compared to other psychedelics, like LSD, psylocibin or DMT, 5-MeO-DMT or a pharmaceutical acceptable salt thereof can be administered to patients suffering from Bipolar Disorder, such as Bipolar I Disorder and Bipolar II Disorder, associated with negative thinking, preferably using dosing schemes as described herein, without a significant risk of inducing mania or hypomania.

Preferably, the patient suffering from Bipolar Disorder, such as Bipolar I Disorder and Bipolar II Disorder, associated with negative thinking does not experience treatment-emergent mania or hypomania.

Postpartum Depression (PPD) is a debilitating mood disorder occurring during pregnancy or within 4 weeks following delivery. While over 50% of women may experience short-lasting low mood or tearfulness after childbirth, a subset of women may develop PPD. Epidemiological studies estimate that the prevalence rate of PPD is about 15%.

The patient suffering from PPD may suffer from a treatment resistant form of the disorder.

Qualitative features such as intense feeling of despair and isolation allows PPD to be distinguished from ‘feeling down’ after recently giving birth.

Feelings of guilt are also particularly implicated in PPD, being significantly predicting of PPD. Potential interventions targeting guilt may be important in the treatment of PPD.

PPD is also known as major depressive disorder with peripartum onset. According to DSM-5 (Diagnostic and Statistical Manual of Mental Disorders, 5th Edition) criteria, PPD is diagnosed when major depressive disorder (MDD) symptoms begin during pregnancy or within four weeks of delivery. Accordingly, PPD patients also suffer from negative feelings such as guilt, hopelessness and worthlessness.

Negative thinking in a patient suffering from PPD may be assessed as part of the determination of the HAM-D, the MADRS or the BPRS. Negative thinking or at least aspects thereof may furthermore be assessed, for instance, by using the State Shame and Guilt Scale (SSGS), the Positive and Negative Affect Schedule-Expanded Form (PANAS-X) or the State Hope Scale (SHS).

In patients with PPD, significant changes in neural activity in brain regions important for self-regulation, empathy, emotion, and cognition are observed. PPD is associated with dysfunctional connectivity of resting state networks, for instance, within and/or between the default mode network and frontoparietal network.

Treating a patient suffering from PPD, including a treatment resistant form of the disorder, and associated negative thinking with 5-MeO-DMT or a pharmaceutically acceptable salt thereof reduces or eliminates the negative thinking and leads to an improvement of PPD.

A patient may suffer from moderate or severe PPD as indicated by a Montgomery-Åsberg Depression Rating Scale (MADRS) score of 20 or more or by a Hamilton Depression Rating Scale (HAM-D) score of 16 or more. It is further considered that the patient may suffer from severe PPD as indicated by a Montgomery-Åsberg Depression Rating Scale (MADRS) score of 35 or more or by a Hamilton Depression Rating Scale (HAM-D) score of 27 or more.

A patient treated according to the invention may have a Montgomery-Åsberg Depression Rating Scale (MADRS) score of 20 or more or a 17-item Hamilton Depression Rating Scale (HAM-D) score of 16 or more.

Further, a patient treated according to the invention may have a MADRS score of 28 or more or a HAM-D score of 22 or more.

Still further, a patient treated according to the invention may have a MADRS score of 35 or more or a HAM-D score of 25 or more.

The reduction or elimination of negative thinking in a patient suffering from PPD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from PPD occurs not later than about 2 hours after the last administration of 5-MeO-DMT or a pharmaceutically acceptable salt thereof. The reduction or elimination of negative thinking in a patient suffering from PPD preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from PPD, in particular of pessimistic thoughts, is reflected by an improvement at least in the score of the MADRS item “pessimistic thoughts” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from PPD, in particular of pessimistic thoughts, as reflected by an improvement in the score of the MADRS item “pessimistic thoughts”, occurs not later than about 2 hours after the last administration of 5-MeO-DMT or a pharmaceutically acceptable salt thereof. The reduction or elimination of negative thinking in a patient suffering from PPD, in particular of pessimistic thoughts, as reflected by an improvement in the score of the MADRS item “pessimistic thoughts”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from PPD, in particular of guilt feelings, is reflected by at least an improvement in the score of the BPRS item “guilt feelings” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from PPD, in particular of guilt feelings, as reflected by an improvement in the score of the BPRS item “guilt feelings”, occurs not later than about 2 hours after the last administration of 5-MeO-DMT or a pharmaceutically acceptable salt thereof. The reduction or elimination of negative thinking in a patient suffering from PPD, in particular of guilt feelings, as reflected by an improvement in the score of the BPRS item “guilt feelings”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from PPD, in particular of depressed mood, is reflected by at least an improvement in the score of the HAM-D item “depressed mood” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from PPD, in particular of depressed mood, as reflected by an improvement in the score of the HAM-D item “depressed mood”, occurs not later than about 2 hours after the last administration of 5-MeO-DMT or a pharmaceutically acceptable salt thereof. The reduction or elimination of negative thinking in a patient suffering from PPD, in particular of depressed mood, as reflected by an improvement in the score of the HAM-D item “depressed mood”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from PPD, in particular of feelings of guilt, is reflected by at least an improvement in the score of the HAM-D item “feelings of guilt” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from PPD, in particular of feelings of guilt, as reflected by an improvement in the score of the HAM-D item “feelings of guilt”, occurs not later than about 2 hours after the last administration of 5-MeO-DMT or a pharmaceutically acceptable salt thereof. The reduction or elimination of negative thinking in a patient suffering from PPD, in particular of feelings of guilt, as reflected by an improvement in the score of the HAM-D item “feelings of guilt”, preferably persists until at least 6 days; in particular until at least 14 days; more preferably until at least 28 days after the last administration of 5-MeO-DMT or a pharmaceutically acceptable salt thereof.

Alternatively or additionally, a reduction or elimination of negative thinking in a patient suffering from PPD, is reflected by at least an improvement in the score of the guilt subscale of the State Shame and Guilt Scale (SSGS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of negative thinking in a patient suffering PPD, as reflected by at least an improvement in the score of the guilt subscale of the State Shame and Guilt Scale (SSGS), occurs not later than about 2 hours after the last administration of 5-MeO-DMT or a pharmaceutically acceptable salt thereof. The reduction or elimination of negative thinking in a patient suffering PPD, as reflected by at least an improvement in the score of the guilt subscale of the State Shame and Guilt Scale (SSGS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from PPD, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from PPD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from PPD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

Alternatively or additionally, the reduction or elimination of negative thinking in a patient suffering from PPD, in particular of hopelessness, is reflected by at least an improvement in the score of the State Hope Scale (SHS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from PPD, in particular of hopelessness, as reflected by at least an improvement in the score of the State Hope Scale (SHS), occurs not later than about 2 hours after the last administration of 5-MeO-DMT or a pharmaceutically acceptable salt thereof. The reduction or elimination of negative thinking in a patient suffering from PPD, in particular of hopelessness, as reflected by at least an improvement in the score of the State Hope Scale (SHS), preferably persists until at least 6 days; in particular until at least 14 days; more preferably until at least 28 days after the last administration of 5-MeO-DMT or a pharmaceutically acceptable salt thereof.

As indicated above, negative thinking is closely linked to PPD. An improvement in negative thinking will therefore also lead to an improvement of PPD. Since negative thinking furthermore also affects other aspects of PPD, the inventors conclude that the improvement in negative thinking, in particular the reduction or elimination of pessimistic thoughts and/or guilt feelings, will additionally contribute to an overall improvement of PPD.

An improvement of PPD in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of PPD in a patient also suffering from associated negative thinking, as reflected by a reduction in the CGI-S score, occurs not later than about 2 hours after the last administration of 5-MeO-DMT or a pharmaceutically acceptable salt thereof. An improvement of PPD in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Negative thinking moreover compromises maternal functioning.

Maternal functioning can, for instance, be assessed using the Barkin Index of Maternal Functioning (BIMF). This index was designed to measure functioning in the year after childbirth. The BIMF is a 20-item self-report measure of functioning. Each item is assigned a score between 0 and 6 so that the maximum total score is 120. The higher the score, the better maternal functioning is rated.

The BIMF identifies the key functional domains of a mother during the postnatal period as: self-care, infant care, mother-child interaction, psychological wellbeing of the mother, social support, management, and adjustment.

A BIMF score of 95 or below is considered herein as representing slightly compromised maternal functioning, a score of 80 or below is considered herein as representing compromised maternal functioning, a score of 65 or below is considered herein as representing severely compromised maternal functioning.

The inventors have determined that increases in the score of the MADRS item “pessimistic thoughts” and/or the BPRS item “guilt feelings” have negative impacts on maternal functioning (maternal competence relating to interactions with the infant(s) as well as maternal self-care).

Increased scores in the MADRS item pessimistic thoughts and/or the BPRS item guilt feelings impair psychological wellbeing, social support and management. Conversely, improvements regarding this MADRS item and/or this BPRS item lead to improvements in maternal functioning, in particular in the BIMF functional domains psychological well-being, social support and/or management.

The inventors conclude that 5-MeO-DMT can be used to treat PPD patients to achieve an improvement of negative thinking, in particular a reduction or elimination of pessimistic thoughts and/or guilt feelings.

The inventors furthermore conclude that a reduction or elimination of pessimistic thoughts and/or guilt feelings by treating a PPD patient does not only lead to a reduction in the MADRS or the BPRS total score, but also to an improvement in maternal functioning, as reflected by an increase in the BIMF score.

The BIMF total score is improved by 10% or more, preferably by 20% or more.

The improvement of maternal functioning in a patient suffering from PPD is reflected by at least an improvement in the BIMF total score on day 7; on day 14; and/or on day 28 after the last administration of 5-MeO-DMT or a pharmaceutically acceptable salt thereof.

The improvement of maternal functioning in a patient suffering from PPD, as reflected by at least an improvement in the BIMF total score, occurs not later than about 6 days after the last administration of 5-MeO-DMT or a pharmaceutically acceptable salt thereof.

The improvement of maternal functioning, as reflected by an improvement in the BIMF total score, preferably persists until at least 14 days; more preferably until at least 28 days after the last administration of 5-MeO-DMT or a pharmaceutically acceptable salt thereof.

Compared to other psychedelics, like LSD, psylocibin or DMT, 5-MeO-DMT or a pharmaceutical acceptable salt thereof can be administered to patients suffering from PPD associated with negative thinking, preferably using dosing schemes as described herein, without a significant risk of inducing mania or hypomania.

Preferably, the patient suffering from PPD associated with negative thinking does not experience treatment-emergent mania or hypomania.

Seasonal Affective Disorder is a mood disorder with seasonal pattern, with symptoms often beginning in autumn and remitting in spring. Many people experience sadness, hopelessness, a loss of interest in activities, fatigue, and social withdrawal.

The patient suffering from Seasonal Affective Disorder may suffer from a treatment resistant form of the disorder.

Seasonal Affective Disorder is associated with negative thinking, including feelings of guilt, hopelessness and worthlessness, because depressive episodes may meet the criteria for major depressive episode according to the DSM-V.

Negative thinking in a patient suffering from Seasonal Affective Disorder may be assessed as part of the determination of the HAM-D, the MADRS or the BPRS. Negative thinking or at least aspects thereof may furthermore be assessed, for instance, by using the State Shame and Guilt Scale (SSGS), the Positive and Negative Affect Schedule-Expanded Form (PANAS-X) or the State Hope Scale (SHS).

Resting state activity involved in sensorimotor, attention, and visual processing is altered in patients with Seasonal Affective Disorder compared to healthy controls.

In patients suffering from Seasonal Affective Disorder altered functional connectivity is observed within and/or between several brain regions implicated in processing, regulation, emotional memory; cognitive processes related to rumination; impaired concentration and physiological arousal. Dysfunctional connectivity is observed within and/or between the DMN, salience network, executive control network and limbic network. Functional connectivity differs significantly from that observed in healthy controls.

Treating a patient suffering from Seasonal Affective Disorder, including a treatment resistant form of the disorder, and associated negative thinking with 5-MeO-DMT or a pharmaceutically acceptable salt thereof reduces or eliminates the negative thinking and leads to an improvement of the Seasonal Affective Disorder.

The reduction or elimination of negative thinking in a patient suffering from Seasonal Affective Disorder is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Seasonal Affective Disorder occurs not later than about 2 hours after the last administration of 5-MeO-DMT or a pharmaceutically acceptable salt thereof. The reduction or elimination of negative thinking in a patient suffering from Seasonal Affective Disorder preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from Seasonal Affective Disorder, in particular of pessimistic thoughts, is reflected by an improvement at least in the score of the MADRS item “pessimistic thoughts” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Seasonal Affective Disorder, in particular of pessimistic thoughts, as reflected by an improvement in the score of the MADRS item “pessimistic thoughts” occurs not later than about 2 hours after the last administration of 5-MeO-DMT or a pharmaceutically acceptable salt thereof. The reduction or elimination of negative thinking in a patient suffering from Seasonal Affective Disorder, in particular of pessimistic thoughts, as reflected by an improvement in the score of the MADRS item “pessimistic thoughts” preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from Seasonal Affective Disorder, in particular of guilt feelings, is reflected by at least an improvement in the score of the BPRS item “guilt feelings” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Seasonal Affective Disorder, in particular of guilt feelings, as reflected by an improvement in the score of the BPRS item “guilt feelings” occurs not later than about 2 hours after the last administration of 5-MeO-DMT or a pharmaceutically acceptable salt thereof. The reduction or elimination of negative thinking in a patient suffering from Seasonal Affective Disorder, in particular of guilt feelings, as reflected by an improvement in the score of the BPRS item “guilt feelings” preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from Seasonal Affective Disorder, in particular of depressed mood, is reflected by at least an improvement in the score of the HAM-D item “depressed mood” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Seasonal Affective Disorder, in particular of depressed mood, as reflected by an improvement in the score of the HAM-D item “depressed mood”, occurs not later than about 2 hours after the last administration of 5-MeO-DMT or a pharmaceutically acceptable salt thereof. The reduction or elimination of negative thinking in a patient suffering from Seasonal Affective Disorder, in particular of depressed mood, as reflected by an improvement in the score of the HAM-D item “depressed mood”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from Seasonal Affective Disorder, in particular of feelings of guilt, is reflected by at least an improvement in the score of the HAM-D item “feelings of guilt” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Seasonal Affective Disorder, in particular of feelings of guilt, as reflected by an improvement in the score of the HAM-D item “feelings of guilt”, occurs not later than about 2 hours after the last administration of 5-MeO-DMT or a pharmaceutically acceptable salt thereof. The reduction or elimination of negative thinking in a patient suffering from Seasonal Affective Disorder, in particular of feelings of guilt, as reflected by an improvement in the score of the HAM-D item “feelings of guilt”, preferably persists until at least 6 days; in particular until at least 14 days; more preferably until at least 28 days after the last administration of 5-MeO-DMT or a pharmaceutically acceptable salt thereof.

Alternatively or additionally, a reduction or elimination of negative thinking in a patient suffering from Seasonal Affective Disorder, is reflected by at least an improvement in the score of the guilt subscale of the State Shame and Guilt Scale (SSGS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of negative thinking in a patient suffering Seasonal Affective Disorder, as reflected by at least an improvement in the score of the guilt subscale of the State Shame and Guilt Scale (SSGS), occurs not later than about 2 hours after the last administration of 5-MeO-DMT or a pharmaceutically acceptable salt thereof. The reduction or elimination of negative thinking in a patient suffering Seasonal Affective Disorder, as reflected by at least an improvement in the score of the guilt subscale of the State Shame and Guilt Scale (SSGS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from Seasonal Affective Disorder, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from Seasonal Affective Disorder, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from Seasonal Affective Disorder, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

Alternatively or additionally, the reduction or elimination of negative thinking in a patient suffering from Seasonal Affective Disorder, in particular of hopelessness, is reflected by at least an improvement in the score of the State Hope Scale (SHS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Seasonal Affective Disorder, in particular of hopelessness, as reflected by at least an improvement in the score of the State Hope Scale (SHS), occurs not later than about 2 hours after the last administration of 5-MeO-DMT or a pharmaceutically acceptable salt thereof. The reduction or elimination of negative thinking in a patient suffering from Seasonal Affective Disorder, in particular of hopelessness, as reflected by at least an improvement in the score of the State Hope Scale (SHS), preferably persists until at least 6 days; in particular until at least 14 days; more preferably until at least 28 days after the last administration of 5-MeO-DMT or a pharmaceutically acceptable salt thereof.

As indicated above, negative thinking is closely linked to Seasonal Affective Disorder. An improvement in negative thinking will therefore also lead to an improvement of Seasonal Affective Disorder. Since negative thinking furthermore also affects other aspects of Seasonal Affective Disorder, the inventors conclude that the improvement in negative thinking will additionally contribute to an overall improvement of Seasonal Affective Disorder.

An improvement of Seasonal Affective Disorder in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of Seasonal Affective Disorder in a patient also suffering from associated negative thinking, as reflected by a reduction in the CGI-S score, occurs not later than about 2 hours after the last administration of 5-MeO-DMT or a pharmaceutically acceptable salt thereof. An improvement of Seasonal Affective Disorder in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Compared to other psychedelics, like LSD, psylocibin or DMT, 5-MeO-DMT or a pharmaceutical acceptable salt thereof can be administered to patients suffering from Seasonal Affective Disorder associated with negative thinking, preferably using dosing schemes as described herein, without a significant risk of inducing mania or hypomania.

Preferably, the patient suffering from Seasonal Affective Disorder associated with negative thinking does not experience treatment-emergent mania or hypomania.

### Persistent Depressive Disorder

Persistent Depressive Disorder, also referred to as dysthymia, is a chronic form of depression. It is diagnosed if depression is present for most of the day for the majority of days over at least a two-year period. Any symptom-free period is less than 2 months.

While depressed, two or more of the following must be present: 1. Hopelessness; 2. Energy low or fatigue; 3. Self-esteem is low; 4. Sleep decreased (insomnia) or increased (hypersomnia): 5. Appetite poor, or overeating; 6. Difficulty making decisions or poor concentration.

The patient suffering from Persistent Depressive Disorder may suffer from a treatment resistant form of the disorder.

According to the DSM-V, Persistent Depressive Disorder can is characterized by the presence, while depressed, of two or more criteria, including feelings of hopelessness. Moreover, the DSM-V notes that criteria for a major depressive disorder may be continuously present for 2 years, which include feelings of worthlessness and excessive or inappropriate guilt.

Negative thinking in a patient suffering from Persistent Depressive Disorder may be assessed as part of the determination of the HAM-D, the MADRS or the BPRS. Negative thinking or at least aspects thereof may furthermore be assessed, for instance, by using the State Shame and Guilt Scale (SSGS), the Positive and Negative Affect Schedule-Expanded Form (PANAS-X) or the State Hope Scale (SHS).

In patients suffering from Persistent Depressive Disorder altered functional connectivity is observed within and/or between several brain regions implicated in processing, regulation, emotional memory; cognitive processes related to rumination; impaired concentration and physiological arousal.

In patients suffering from Persistent Depressive Disorder altered functional connectivity is observed within and/or between several brain regions implicated in processing, regulation, emotional memory; cognitive processes related to rumination; impaired concentration and physiological arousal. Dysfunctional connectivity is observed within and/or between the DMN, salience network, executive control network and limbic network. Functional connectivity differs significantly from that observed in healthy controls.

Treating a patient suffering from Persistent Depressive Disorder, including a treatment resistant form of the disorder, and associated negative thinking with 5-MeO-DMT or a pharmaceutically acceptable salt thereof reduces or eliminates the negative thinking and leads to an improvement of Persistent Depressive Disorder.

The reduction or elimination of negative thinking in a patient suffering from Persistent Depressive Disorder is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Persistent Depressive Disorder occurs not later than about 2 hours after the last administration of 5-MeO-DMT or a pharmaceutically acceptable salt thereof. The reduction or elimination of negative thinking in a patient suffering from Persistent Depressive Disorder preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from Persistent Depressive Disorder, in particular of pessimistic thoughts, is reflected by an improvement at least in the score of the MADRS item “pessimistic thoughts” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Persistent Depressive Disorder, in particular of pessimistic thoughts, as reflected by an improvement in the score of the MADRS item “pessimistic thoughts” occurs not later than about 2 hours after the last administration of 5-MeO-DMT or a pharmaceutically acceptable salt thereof. The reduction or elimination of negative thinking in a patient suffering from Persistent Depressive Disorder, in particular of pessimistic thoughts, as reflected by an improvement in the score of the MADRS item “pessimistic thoughts”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from Persistent Depressive Disorder, in particular of guilt feelings, is reflected by at least an improvement in the score of the BPRS item “guilt feelings” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Persistent Depressive Disorder, in particular of guilt feelings, as reflected by an improvement in the score of the BPRS item “guilt feelings”, occurs not later than about 2 hours after the last administration of 5-MeO-DMT or a pharmaceutically acceptable salt thereof. The reduction or elimination of negative thinking in a patient suffering from Persistent Depressive Disorder, in particular of guilt feelings, as reflected by an improvement in the score of the BPRS item “guilt feelings”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from Persistent Depressive Disorder, in particular of depressed mood, is reflected by at least an improvement in the score of the HAM-D item “depressed mood” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Persistent Depressive Disorder, in particular of depressed mood, as reflected by an improvement in the score of the HAM-D item “depressed mood”, occurs not later than about 2 hours after the last administration of 5-MeO-DMT or a pharmaceutically acceptable salt thereof. The reduction or elimination of negative thinking in a patient suffering from Persistent Depressive Disorder, in particular of depressed mood, as reflected by an improvement in the score of the HAM-D item “depressed mood”, preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from Persistent Depressive Disorder, in particular of feelings of guilt, is reflected by at least an improvement in the score of the HAM-D item “feelings of guilt” about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Persistent Depressive Disorder, in particular of feelings of guilt, as reflected by an improvement in the score of the HAM-D item “feelings of guilt”, occurs not later than about 2 hours after the last administration of 5-MeO-DMT or a pharmaceutically acceptable salt thereof. The reduction or elimination of negative thinking in a patient suffering from Persistent Depressive Disorder, in particular of feelings of guilt, as reflected by an improvement in the score of the HAM-D item “feelings of guilt”, preferably persists until at least 6 days; in particular until at least 14 days; more preferably until at least 28 days after the last administration of 5-MeO-DMT or a pharmaceutically acceptable salt thereof.

Alternatively or additionally, a reduction or elimination of negative thinking in a patient suffering from Persistent Depressive Disorder, is reflected by at least an improvement in the score of the guilt subscale of the State Shame and Guilt Scale (SSGS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of negative thinking in a patient suffering Persistent Depressive Disorder, as reflected by at least an improvement in the score of the guilt subscale of the State Shame and Guilt Scale (SSGS), occurs not later than about 2 hours after the last administration of 5-MeO-DMT or a pharmaceutically acceptable salt thereof. The reduction or elimination of negative thinking n a patient suffering Persistent Depressive Disorder, as reflected by at least an improvement in the score of the guilt subscale of the State Shame and Guilt Scale (SSGS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from Persistent Depressive Disorder, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from Persistent Depressive Disorder, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from Persistent Depressive Disorder, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

Alternatively or additionally, the reduction or elimination of negative thinking in a patient suffering from Persistent Depressive Disorder, in particular of hopelessness, is reflected by at least an improvement in the score of the State Hope Scale (SHS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Persistent Depressive Disorder, in particular of hopelessness, as reflected by at least an improvement in the score of the State Hope Scale (SHS), occurs not later than about 2 hours after the last administration of 5-MeO-DMT or a pharmaceutically acceptable salt thereof. The reduction or elimination of negative thinking in a patient suffering from Persistent Depressive Disorder, in particular of hopelessness, as reflected by at least an improvement in the score of the State Hope Scale (SHS), preferably persists until at least 6 days; in particular until at least 14 days; more preferably until at least 28 days after the last administration of 5-MeO-DMT or a pharmaceutically acceptable salt thereof.

As indicated above, negative thinking is closely linked to Persistent Depressive Disorder. An improvement in negative thinking will therefore also lead to an improvement of Persistent Depressive Disorder. Since negative thinking furthermore also affects other aspects of Persistent Depressive Disorder, the inventors conclude that the improvement in negative thinking will additionally contribute to an overall improvement of Persistent Depressive Disorder.

An improvement of Persistent Depressive Disorder in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of Persistent Depressive Disorder in a patient also suffering from associated negative thinking, as reflected by a reduction in the CGI-S score, occurs not later than about 2 hours after the last administration of 5-MeO-DMT or a pharmaceutically acceptable salt thereof. An improvement of Persistent Depressive Disorder in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Compared to other psychedelics, like LSD, psylocibin or DMT, 5-MeO-DMT or a pharmaceutical acceptable salt thereof can be administered to patients suffering from Persistent Depressive Disorder associated with s negative thinking, preferably using dosing schemes as described herein, without a significant risk of inducing mania or hypomania.

Preferably, the patient suffering from Persistent Depressive Disorder associated with negative thinking does not experience treatment-emergent mania or hypomania.

### Anxiety Disorder

An Anxiety Disorder is a type of mental health condition. Symptoms include feelings of nervousness, panic and fear as well as sweating and a rapid heartbeat. Anxiety is linked to fear and manifests as a future-oriented mood state that consists of a complex cognitive, affective, physiological, and behavioural response system associated with preparation for the anticipated events or circumstances perceived as threatening.

The patient suffering from Anxiety Disorder may suffer from a treatment resistant form of the disorder.

Negative thinking or at least aspects thereof may be assessed, for instance, by using the Positive and Negative Affect Schedule-Expanded Form (PANAS-X).

Anxiety Disorders are associated with alterations in the functional connectivity of resting state networks. Anxiety Disorders show abnormalities in the default mode network (DMN) affecting the sense of self, the salience network (SN) controlling emotion/anxiety and the somatomotor network (SMN) responsible for bodily awareness. The resting state balance within and/or between each of these networks, e.g., SMN and SN, relative to the DMN may be abnormal in the different anxiety disorders.

Treating a patient suffering from an Anxiety Disorder, including a treatment resistant form of the disorder, and associated negative thinking with 5-MeO-DMT or a pharmaceutically acceptable salt thereof reduces or eliminates the negative thinking and leads to an improvement of the Anxiety Disorder.

The reduction or elimination of negative thinking in a patient suffering from an Anxiety Disorder is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from an Anxiety Disorder occurs not later than about 2 hours after the last administration of 5-MeO-DMT or a pharmaceutically acceptable salt thereof. The reduction or elimination of negative thinking in a patient suffering from an Anxiety Disorder preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from an Anxiety Disorder, is reflected by at least an improvement in the score of one or more of the basic negative emotion scales of the Positive and Negative Affect Schedule-Expanded Form (PANAS-X) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from an Anxiety Disorder, as reflected by at least an improvement in the score of one or more of the basic negative emotion scales of the Positive and Negative Affect Schedule-Expanded Form (PANAS-X), occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from an Anxiety Disorder, as reflected by at least an improvement in the score of one or more of the basic negative emotion scales of the Positive and Negative Affect Schedule-Expanded Form (PANAS-X), preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

As indicated above, negative thinking occurs in patients with an Anxiety Disorder. An improvement in negative thinking will therefore also lead to an improvement of Anxiety Disorder. Since negative thinking furthermore also affects other aspects of Anxiety Disorder, the inventors conclude that the improvement in negative thinking will additionally contribute to an overall improvement of Anxiety Disorder.

An improvement of the Anxiety Disorder in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the Anxiety Disorder in a patient also suffering from associated negative thinking, as reflected by a reduction in the CGI-S score, occurs not later than about 2 hours after the last administration of 5-MeO-DMT or a pharmaceutically acceptable salt thereof. An improvement of the Anxiety Disorder in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Generalised Anxiety Disorder (GAD) is characterized by persistent and excessive, difficult to control worry about a wide range of situations and issues. Patients suffering from GAD may anticipate disaster and may be overly concerned about money, health, family, work, or other issues.

Generalized anxiety disorder is diagnosed when an individual experiences persistent worry about everyday challenges out of proportion to the perceived threat. Patients with GAD usually experience excessive fear that can last months to years.

GAD interferes with social, occupational, or other important areas of functioning.

The patient suffering from GAD may suffer from a treatment resistant form of the disorder.

GAD is associated with poor control over intense negative emotions, which in turn may lead to greater impulsivity in patients with GAD.

Shame is an important symptom in GAD. Shame-proneness is found to have a significant relationship with both GAD and social anxiety disorder. It is thought that worry allows patients with GAD to avoid shameful emotions towards the self.

The relevance of negative thinking in patients suffering from GAD is also reflected in, for example, the Generalised Anxiety Disorder Screener (GAD-7), containing different items relating to feelings of worry.

Negative thinking or at least aspects thereof may be assessed, for instance, by using the Positive and Negative Affect Schedule-Expanded Form (PANAS-X).

Patients with GAD show also altered functional connectivity, especially within the default mode network.

Treating a patient suffering from GAD, including a treatment resistant form of the disorder, and associated negative thinking with 5-MeO-DMT or a pharmaceutically acceptable salt thereof reduces or eliminates the negative thinking and leads to an improvement of GAD.

The reduction or elimination of negative thinking in a patient suffering from GAD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from GAD occurs not later than about 2 hours after the last administration of 5-MeO-DMT or a pharmaceutically acceptable salt thereof. The reduction or elimination of negative thinking in a patient suffering from GAD preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from GAD, is reflected by at least an improvement in the score of one or more of the basic negative emotion scales of the Positive and Negative Affect Schedule-Expanded Form (PANAS-X) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from GAD, as reflected by at least an improvement in the score of one or more of the basic negative emotion scales of the Positive and Negative Affect Schedule-Expanded Form (PANAS-X), occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from GAD, as reflected by at least an improvement in the score of one or more of the basic negative emotion scales of the Positive and Negative Affect Schedule-Expanded Form (PANAS-X), preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

As indicated above, negative thinking is a relevant aspect in patients with GAD. An improvement in negative thinking will therefore also lead to an improvement of GAD. Since negative thinking furthermore also affects other aspects of GAD, the inventors conclude that the improvement in negative thinking will additionally contribute to an overall improvement of GAD.

An improvement of the GAD in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the GAD in a patient also suffering from associated negative thinking, as reflected by a reduction in the CGI-S score, occurs not later than about 2 hours after the last administration of 5-MeO-DMT or a pharmaceutically acceptable salt thereof. An improvement of the GAD in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Social Anxiety Disorder (SAD), also called social phobia, is one of the most common types of anxiety. SAD is characterized by intense anxiety or fear of being judged, negatively evaluated, or rejected in a social or performance situation. This often leads to avoidance of the social situation and can cause impairments in school, work, or relationships.

SAD is characterized by intense anxiety or fear of being judged, negatively evaluated, or rejected in a social or performance situation.

The patient suffering from SAD may suffer from a treatment resistant form of the disorder.

Patients with SAD experience a high intensity of negative emotions as well as a greater negative emotional response compared to age-matched individuals.

Shame is an important symptom in SAD. Shame-proneness is found to have a significant relationship with both SAD and generalised anxiety disorder. It is thought that negative representation of the self is a key factor in maintaining SAD symptoms.

Negative thinking or at least aspects thereof may be assessed, for instance, by using the Positive and Negative Affect Schedule-Expanded Form (PANAS-X).

In patients suffering from SAD, altered functional connectivity is observed in the default mode network and the salience network.

Treating a patient suffering from SAD, including a treatment resistant form of the disorder, and associated negative thinking with 5-MeO-DMT or a pharmaceutically acceptable salt thereof reduces or eliminates the negative thinking and leads to an improvement of SAD.

The reduction or elimination of negative thinking in a patient suffering from SAD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from SAD occurs not later than about 2 hours after the last administration of 5-MeO-DMT or a pharmaceutically acceptable salt thereof. The reduction or elimination of negative thinking in a patient suffering from SAD preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from SAD, is reflected by at least an improvement in the score of one or more of the basic negative emotion scales of the Positive and Negative Affect Schedule-Expanded Form (PANAS-X) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from SAD, as reflected by at least an improvement in the score of one or more of the basic negative emotion scales of the Positive and Negative Affect Schedule-Expanded Form (PANAS-X), occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from SAD, as reflected by at least an improvement in the score of one or more of the basic negative emotion scales of the Positive and Negative Affect Schedule-Expanded Form (PANAS-X), preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

As indicated above, negative thinking occurs in patients with SAD. An improvement in negative thinking will therefore also lead to an improvement of SAD. Since negative thinking furthermore also affects other aspects of SAD, the inventors conclude that the improvement in negative thinking will additionally contribute to an overall improvement of SAD.

An improvement of SAD in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of SAD in a patient also suffering from associated negative thinking, as reflected by a reduction in the CGI-S score, occurs not later than about 2 hours after the last administration of 5-MeO-DMT or a pharmaceutically acceptable salt thereof. An improvement of the SAD in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

### Obsessive Compulsive and Related Disorders

Obsessive Compulsive Disorder (OCD) is a mental illness that causes repeated unwanted thoughts or sensations (obsessions) or the urge to do something over and over again (compulsions). Patients may suffer from both obsessions and compulsions.

The patient suffering from OCD may suffer from a treatment resistant form of the disorder.

Patients with OCD show greater levels of distress when prompted with the same stimuli as their siblings or other matched controls. It is known that emotional dysregulation is a feature of OCD.

Guilt has been identified as a phenomenological feature of OCD, and is thought to play a perpetuating role in OCD symptoms. Pathological guilt is a very prominent feature in certain subtypes of OCD where common rituals performed may include prayer, confession and excessive washing.

Negative thinking or at least aspects thereof may be assessed, for instance, by using the State Shame and Guilt Scale (SSGS) or the Positive and Negative Affect Schedule-Expanded Form (PANAS-X).

The relevance of negative feelings, including feelings of guilt, is supported by administering the Moral Orientation Guilt Scale (MOGS) on OCD patients. The MOGS is a 17-items containing empirical tool allowing the measurement of different types of guilt according to moral orientation.

Neuroimaging studies using functional magnetic resonance imaging of patients suffering from OCD show functional connectivity alterations within and/or between frontoparietal network, salience network, and default mode network.

Treating a patient suffering from OCD, including a treatment resistant form of the disorder, and associated negative thinking with 5-MeO-DMT or a pharmaceutically acceptable salt thereof reduces or eliminates the negative thinking and leads to an improvement of OCD.

The reduction or elimination of negative thinking in a patient suffering from OCD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from OCD occurs not later than about 2 hours after the last administration of 5-MeO-DMT or a pharmaceutically acceptable salt thereof. The reduction or elimination of negative thinking in a patient suffering from OCD preferably persists until at least 6 days; in particular until at least 14 days; more preferably until at least 28 days after the last administration of 5-MeO-DMT or a pharmaceutically acceptable salt thereof.

A reduction or elimination of negative thinking in a patient suffering from OCD, in particular of guilt feelings, is reflected by at least an improvement in the score of the guilt subscale of the State Shame and Guilt Scale (SSGS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of negative thinking in a patient suffering from OCD, in particular of guilt feelings, as reflected by at least an improvement in the score of the guilt subscale of the State Shame and Guilt Scale (SSGS), occurs not later than about 2 hours after the last administration of 5-MeO-DMT or a pharmaceutically acceptable salt thereof. The reduction or elimination of negative thinking in a patient suffering from OCD, in particular of guilt feelings, as reflected by at least an improvement in the score of the guilt subscale of the State Shame and Guilt Scale (SSGS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from OCD, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from OCD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from OCD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

Alternatively or additionally, the reduction or elimination of negative thinking in a patient suffering from OCD, in particular of guilt feelings, is reflected by at least an improvement in the Moral Orientation Guilt Scale (MOGS) score or in one or more of its component scores about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from OCD, in particular of guilt feelings, as reflected by an improvement in the MOGS score or in one or more of its component scores occurs not later than about 2 hours after the last administration of 5-MeO-DMT or a pharmaceutically acceptable salt thereof. The reduction or elimination of negative thinking in a patient suffering from OCD, in particular of guilt feelings, as reflected by an improvement in the MOGS score or in one or more of its component scores, preferably persists until at least 6 days; in particular until at least 14 days; more preferably until at least 28 days after the last administration of 5-MeO-DMT or a pharmaceutically acceptable salt thereof.

As indicated above, negative thinking is closely linked to OCD. An improvement in negative thinking will therefore also lead to an improvement of OCD. Since negative thinking furthermore also affects other aspects of OCD, the inventors conclude that the improvement in negative thinking will additionally contribute to an overall improvement of OCD.

An improvement of the OCD in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the OCD in a patient also suffering from associated negative thinking, as reflected by a reduction in the CGI-S score, occurs not later than about 2 hours after the last administration of 5-MeO-DMT or a pharmaceutically acceptable salt thereof. An improvement of the OCD in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Body Dysmorphic Disorder (BDD) patients misperceive defects in their appearance, disrupting their ability to function in their daily lives, with disturbing preoccupations, ritualistic behaviours, and emotional distress.

The patient suffering from BDD may suffer from a treatment resistant form of the disorder.

Intense shame concerning one's body is a core component of BDD. Internalised shame is greater in patients with BDD compared to OCD and healthy controls. Greater internalised shame was also correlated with greater BDD severity and poorer insight.

Negative thinking or at least aspects thereof may be assessed, for instance, by using the Positive and Negative Affect Schedule-Expanded Form (PANAS-X).

Functional magnetic resonance imaging of patients suffering from BDD reveals alterations within and/or between certain brain areas located in the default mode network, the dorsal attention network and the salience network.

Treating a patient suffering from BDD, including a treatment resistant form of the disorder, and associated negative thinking with 5-MeO-DMT or a pharmaceutically acceptable salt thereof reduces or eliminates the negative thinking and leads to an improvement of the BDD.

The reduction or elimination of negative thinking in a patient suffering from BDD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from BDD occurs not later than about 2 hours after the last administration of 5-MeO-DMT or a pharmaceutically acceptable salt thereof. The reduction or elimination of negative thinking in a patient suffering from BDD preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from BDD, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from BDD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from BDD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

As indicated above, negative thinking is an important aspect in patients suffering from BDD. An improvement in negative thinking will therefore also lead to an improvement of BDD. Since negative thinking furthermore also affects other aspects of BDD, the inventors conclude that the improvement in negative thinking will additionally contribute to an overall improvement of the BDD.

An improvement of the BDD in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the BDD in a patient also suffering from associated negative thinking, as reflected by a reduction in the CGI-S score, occurs not later than about 2 hours after the last administration of 5-MeO-DMT or a pharmaceutically acceptable salt thereof. An improvement of the BDD in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

### Post-Traumatic Stress Disorder (PTSD)

Post-Traumatic Stress Disorder (PTSD) is a mental health condition that can develop based on a terrifying event—either experienced or witnessed by the patient. Symptoms may include flashbacks, nightmares and severe anxiety, as well as uncontrollable thoughts about the event.

A patient suffering from PTSD may suffer from a treatment resistant form of the disorder.

The DSM-V recognises negative emotions such as guilt as evidence for negative alterations in cognition or mood associated with the traumatic event. ‘Persistent negative emotional state’ of which guilt is listed as an example forms part of criterion D for the diagnosis of PTSD.

PTSD symptoms are associated with difficulty in regulating negative emotions. Functional MRI results show decreased activation of brain areas associated with top-down regulation of emotions and increased activation of areas such as the amygdala in brains of patients with PTSD when presented with negative stimuli, providing neuroimaging evidence of impaired ability to down-regulate negative emotion.

Analysis of resting state functional magnetic resonance imaging in patients suffering from PTSD reveals alterations within and/or between regions located in the default mode network and salience network.

Negative thinking is an important aspect in patients suffering from PTSD. This is supported by the PTSD checklist for DSM-5 (PCL-5) and the Trauma Symptom Checklist-40 both containing items relating to negative feelings of guilt.

Negative thinking or at least aspects thereof may be assessed, for instance, by using the State Shame and Guilt Scale (SSGS) or the Positive and Negative Affect Schedule-Expanded Form (PANAS-X).

Treating a patient suffering from PTSD, including a treatment resistant form of the disorder, and associated negative thinking with 5-MeO-DMT or a pharmaceutically acceptable salt thereof reduces or eliminates the negative thinking and leads to an improvement of PTSD.

The reduction or elimination of negative thinking in a patient suffering from PTSD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from PTSD occurs not later than about 2 hours after the last administration of 5-MeO-DMT or a pharmaceutically acceptable salt thereof. The reduction or elimination of negative thinking in a patient suffering from PTSD preferably persists until at least 6 days; in particular until at least 14 days; more preferably until at least 28 days after the last administration of 5-MeO-DMT or a pharmaceutically acceptable salt thereof.

A reduction or elimination of negative thinking in a patient suffering from PTSD, in particular of guilt feelings, is reflected by at least an improvement in the score of the guilt subscale of the State Shame and Guilt Scale (SSGS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of negative thinking in a patient suffering from PTSD, in particular of guilt feelings, as reflected by at least an improvement in the score of the guilt subscale of the State Shame and Guilt Scale (SSGS) occurs not later than about 2 hours after the last administration of 5-MeO-DMT or a pharmaceutically acceptable salt thereof. The reduction or elimination of negative thinking in a patient suffering from PTSD, in particular of guilt feelings, as reflected by at least an improvement in the score of the guilt subscale of the State Shame and Guilt Scale (SSGS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from PTSD, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from PTSD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from PTSD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

As indicated above, negative thinking is a very important aspect in patients suffering from PTSD. An improvement in negative thinking will therefore also lead to an improvement of PTSD. Since negative thinking furthermore also affects other aspects of PTSD, the inventors conclude that the improvement in negative thinking will additionally contribute to an overall improvement of PTSD.

An improvement of the PTSD in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the PTSD in a patient also suffering from associated negative thinking, as reflected by a reduction in the CGI-S score, occurs not later than about 2 hours after the last administration of 5-MeO-DMT or a pharmaceutically acceptable salt thereof. An improvement of the PTSD in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

### Pain Disorders

Chronic Pain, also referred to as persistent pain, is long standing pain that persists beyond the usual recovery period, for instance, after an injury or operation, despite medication or treatment. Patients may also suffer from Chronic Pain without any apparent cause, such as a history of an injury or operation.

Chronic Pain interferes with quality of life, mental and emotional health. Negative thinking occurs in patients suffering from pain and has been associated with Chronic Pain.

A patient suffering from Chronic Pain may suffer from a treatment resistant form of the disorder.

Emotion cannot be separated from the concept of chronic pain in the biopsychosocial model of pain perception.

Increased negative emotions were strongly associated with increased pain and impaired functioning, suggesting a role for negative emotions in the perception of pain as well as daily functioning.

Health-related guilt has also been shown to be an important psychological factor in patients with chronic pain, and is strongly associated with more pain and poorer function in this patient population.

The emotional component in Chronic Pain has been investigated by using the Minnesota Multiphasic Personality Inventory (MMPI), which contains 19 clinical scales including a depression scale measuring clinical depression, which is characterised for example by poor morale and a lack of hope in the future. This indicates that negative thinking is an accepted aspect of Chronic Pain.

Negative thinking or at least aspects thereof may be assessed, for instance, by using the State Shame and Guilt Scale (SSGS), the State Hope Scale (SHS) or the Positive and Negative Affect Schedule-Expanded Form (PANAS-X).

Chronic Pain patients display brain alterations regarding brain function and structure. These changes are related to the persistence of pain, long after the initial nociceptive input has disappeared. Resting state functional magnetic resonance imaging reveals alterations in distinct regions within and/or between the default mode network, the somatomotor/sensorimotor network, and the salience network.

Treating a patient suffering from Chronic Pain, including a treatment resistant form of the disorder, and associated negative thinking with 5-MeO-DMT or a pharmaceutically acceptable salt thereof reduces or eliminates the negative thinking and leads to an improvement of Chronic Pain.

The reduction or elimination of negative thinking in a patient suffering from Chronic Pain is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Chronic Pain occurs not later than about 2 hours after the last administration of 5-MeO-DMT or a pharmaceutically acceptable salt thereof. The reduction or elimination of negative thinking in a patient suffering from Chronic Pain preferably persists until at least 6 days; in particular until at least 14 days; more preferably until at least 28 days after the last administration of 5-MeO-DMT or a pharmaceutically acceptable salt thereof.

A reduction or elimination of negative thinking in a patient suffering from Chronic Pain, in particular of guilt feelings, is reflected by at least an improvement in the score of the guilt subscale of the State Shame and Guilt Scale (SSGS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of negative thinking in a patient suffering from Chronic Pain, in particular of guilt feelings, as reflected by at least an improvement in the score of the guilt subscale of the State Shame and Guilt Scale (SSGS), occurs not later than about 2 hours after the last administration of 5-MeO-DMT or a pharmaceutically acceptable salt thereof. The reduction or elimination of negative thinking in a patient suffering from Chronic Pain, in particular of guilt feelings, as reflected by at least an improvement in the score of the guilt subscale of the State Shame and Guilt Scale (SSGS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from Chronic Pain, in particular of hopelessness, is reflected by at least an improvement in the score of the State Hope Scale (SHS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Chronic Pain, in particular of hopelessness, as reflected by at least an improvement in the score of the State Hope Scale (SHS), occurs not later than about 2 hours after the last administration of 5-MeO-DMT or a pharmaceutically acceptable salt thereof. The reduction or elimination of negative thinking in a patient suffering from Chronic Pain, in particular of hopelessness, as reflected by at least an improvement in the score of the State Hope Scale (SHS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from Chronic Pain, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from Chronic Pain, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from Chronic Pain, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

As indicated above, negative thinking is an important aspect in patients with Chronic Pain. An improvement in negative thinking will therefore also lead to an improvement of Chronic Pain. Since negative thinking furthermore also affects other aspects of Chronic Pain, the inventors conclude that the improvement in negative thinking will additionally contribute to an overall improvement of Chronic Pain.

An improvement of the Chronic Pain in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the Chronic Pain in a patient also suffering from associated negative thinking, as reflected by a reduction in the CGI-S score, occurs not later than about 2 hours after the last administration of 5-MeO-DMT or a pharmaceutically acceptable salt thereof. An improvement of the Chronic Pain in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

### Mental and Behavioural Disorders due to Psychoactive Substance Use

Substance Use Disorder (SUD) is a mental disorder that affects a person's behaviour, leading to a person's inability to control their use of substances such as legal or illegal drugs, alcohol, or medications. Symptoms can range from moderate to severe, with addiction being the most severe form of SUD.

A patient suffering from SUD may suffer from a treatment resistant form of the disorder.

Negative emotions play a central role in SUD, whereby negative feedback is one mechanism which maintains substance use behaviour as the drug is used to escape or withdraw from negative emotional states.

Shame is an important emotion in SUD and has been found to impair reduction in substance misuse as well be positively correlated to increased substance misuse. This effect may be mediated by the shame addiction cycle where the presence of shame resultant from substance misuse is a negative affective state that can trigger further substance use.

The feeling of guilt is also common in patients with SUD and can interfere with self-supporting attitudes and actions necessary to reduce addiction behaviours.

In patients suffering from SUD, deficits in cognitive control are associated with altered connectivity within and/or between resting state networks, such as the default mode network, the salience network, the central executive network, the limbic network and the reward network.

Negative thinking or at least aspects thereof may be assessed, for instance, by using the State Shame and Guilt Scale (SSGS) or the Positive and Negative Affect Schedule-Expanded Form (PANAS-X).

Treating a patient suffering from SUD, including a treatment resistant form of the disorder, and associated negative thinking with 5-MeO-DMT or a pharmaceutically acceptable salt thereof reduces or eliminates the negative thinking and leads to an improvement of SUD.

The reduction or elimination of negative thinking in a patient suffering from SUD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from SUD occurs not later than about 2 hours after the last administration of 5-MeO-DMT or a pharmaceutically acceptable salt thereof. The reduction or elimination of negative thinking in a patient suffering from SUD preferably persists until at least 6 days; in particular until at least 14 days; more preferably until at least 28 days after the last administration of 5-MeO-DMT or a pharmaceutically acceptable salt thereof.

A reduction or elimination of negative thinking in a patient suffering from SUD, in particular of guilt feelings, is reflected by at least an improvement in the score of the guilt subscale of the State Shame and Guilt Scale (SSGS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of negative thinking in a patient suffering from SUD, in particular of guilt feelings, as reflected by at least an improvement in the score of the guilt subscale of the State Shame and Guilt Scale (SSGS), occurs not later than about 2 hours after the last administration of 5-MeO-DMT or a pharmaceutically acceptable salt thereof. The reduction or elimination of negative thinking in a patient suffering from SUD, in particular of guilt feelings, as reflected by at least an improvement in the score of the guilt subscale of the State Shame and Guilt Scale (SSGS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from SUD, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from SUD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from SUD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

As indicated above, negative thinking is an important aspect in patients suffering from SUD. An improvement in negative thinking will therefore also lead to an improvement of SUD. Since negative thinking furthermore also affects other aspects of SUD, the inventors conclude that the improvement in negative thinking will additionally contribute to an overall improvement of Substance Use Disorder.

An improvement of the SUD in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the SUD in a patient also suffering from associated negative thinking, as reflected by a reduction in the CGI-S score, occurs not later than about 2 hours after the last administration of 5-MeO-DMT or a pharmaceutically acceptable salt thereof. An improvement of the SUD in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

### Psychotic Disorders

Psychotic Disorders are severe mental disorders that cause abnormal thinking and perceptions. Psychotic Disorders are characterised by significant impairments in reality testing and alterations in behaviour manifest in positive symptoms such as persistent delusions, persistent hallucinations, disorganised thinking (typically manifest as disorganised speech), grossly disorganised behaviour, and experiences of passivity and control, negative symptoms such as blunted or flat affect and avolition, and psychomotor disturbances.

A patient suffering from a Psychotic Disorder may suffer from a treatment resistant form of the disorder.

Cognitive models of hallucinations and delusions in psychotic disorders emphasise negative emotions as central in the development and maintenance of psychosis.

Negative affect may precede and directly mediate psychotic episodes in people vulnerable to psychosis. Compared to healthy controls, patients with psychosis exhibit greater difficulties with emotional regulation.

Negative thinking or at least aspects thereof may be assessed, for instance, by using the Positive and Negative Affect Schedule-Expanded Form (PANAS-X).

Brain imaging of patients suffering from psychosis by functional magnetic resonance imaging of brain resting state networks, reveals profound alterations in distinct regions within and/or between the central executive network, the default mode network and the salience network. Even in patient populations at risk for psychosis, alterations in resting state networks can be identified.

Treating a patient suffering from a Psychotic Disorder, including a treatment resistant form of the disorder, and associated negative thinking with 5-MeO-DMT or a pharmaceutically acceptable salt thereof reduces or eliminates the negative thinking and leads to an improvement of the Psychotic Disorder.

The reduction or elimination of negative thinking in a patient suffering from a Psychotic Disorder is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from a Psychotic Disorder occurs not later than about 2 hours after the last administration of 5-MeO-DMT or a pharmaceutically acceptable salt thereof. The reduction or elimination of negative thinking in a patient suffering from a Psychotic Disorder preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from a Psychotic Disorder, is reflected by at least an improvement in the score of one or more of the basic negative emotion scales of the Positive and Negative Affect Schedule-Expanded Form (PANAS-X), about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from a Psychotic Disorder, as reflected by at least an improvement in the score of one or more of the basic negative emotion scales of the Positive and Negative Affect Schedule-Expanded Form (PANAS-X), occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from a Psychotic Disorder, as reflected by at least an improvement in the score of one or more of the basic negative emotion scales of the Positive and Negative Affect Schedule-Expanded Form (PANAS-X), preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

As indicated above, the occurrence of negative thinking is an important disease aspect in patients suffering from a Psychotic Disorder. An improvement in negative thinking will therefore also lead to an improvement of the Psychotic Disorder. Since negative thinking furthermore also affects other aspects of a Psychotic Disorder, the inventors conclude that the improvement in negative thinking will additionally contribute to an overall improvement of the Psychotic Disorder.

An improvement of a Psychotic Disorder in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of a Psychotic Disorder in a patient also suffering from associated negative thinking, as reflected by a reduction in the CGI-S score, occurs not later than about 2 hours after the last administration of 5-MeO-DMT or a pharmaceutically acceptable salt thereof. An improvement of the Psychotic Disorder in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Compared to other psychedelics, like LSD, psylocibin or DMT, 5-MeO-DMT or a pharmaceutical acceptable salt thereof can be administered to patients suffering from a Psychotic Disorder characterised by mania or hypomania and associated with negative thinking, preferably using dosing schemes as described herein, without a significant risk of inducing mania or hypomania.

Preferably, the patient suffering from a Psychotic Disorder characterised by mania or hypomania and associated with negative thinking does not experience treatment-emergent mania or hypomania.

Schizophrenia is a severe mental health condition characterised by disturbances in multiple mental modalities, including thinking, perception, self-experience, cognition, volition, affect and behaviour. Psychomotor disturbances, including catatonia, may be present.

A patient suffering from Schizophrenia may suffer from a treatment resistant form of the disorder.

Cognitive models of hallucinations and delusions in psychotic disorders emphasise negative emotions as central in the development and maintenance of psychosis.

Guilt is often present in patients with Schizophrenia, particularly in those with Asian backgrounds. Guilt has been shown to increase with the chronicity of disease.

Abnormal resting state functional connectivity, particularly within and/or between the default mode network, the frontoparietal network and the salience network, is reported in individuals with Schizophrenia. In individuals at high risk for psychosis and in those diagnosed with schizophrenia, several studies have found associations between sleep disturbances and symptom severity.

Negative thinking or at least aspects thereof may be assessed, for instance, by using the State Shame and Guilt Scale (SSGS) or the Positive and Negative Affect Schedule-Expanded Form (PANAS-X).

Treating a patient suffering from Schizophrenia, including a treatment resistant form of the disorder, and associated negative thinking with 5-MeO-DMT or a pharmaceutically acceptable salt thereof reduces or eliminates the negative thinking and leads to an improvement of Schizophrenia.

The reduction or elimination of negative thinking in a patient suffering from Schizophrenia is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Schizophrenia occurs not later than about 2 hours after the last administration of 5-MeO-DMT or a pharmaceutically acceptable salt thereof. The reduction or elimination of negative thinking in a patient suffering from Schizophrenia preferably persists until at least 6 days; in particular until at least 14 days; more preferably until at least 28 days after the last administration of 5-MeO-DMT or a pharmaceutically acceptable salt thereof.

A reduction or elimination of negative thinking in a patient suffering from Schizophrenia, in particular of guilt feelings, is reflected by at least an improvement in the score of the guilt subscale of the State Shame and Guilt Scale (SSGS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of negative thinking in a patient suffering from Schizophrenia, in particular of guilt feelings, as reflected by at least an improvement in the score of the guilt subscale of the State Shame and Guilt Scale (SSGS), occurs not later than about 2 hours after the last administration of 5-MeO-DMT or a pharmaceutically acceptable salt thereof. The reduction or elimination of negative thinking in a patient suffering from Schizophrenia, in particular of guilt feelings, as reflected by at least an improvement in the score of the guilt subscale of the State Shame and Guilt Scale (SSGS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from Schizophrenia, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from Schizophrenia, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from Schizophrenia, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

As indicated above, negative thinking is closely linked to Schizophrenia. An improvement in negative thinking will therefore also lead to an improvement of the Schizophrenia. Since negative thinking furthermore also affects other aspects of Schizophrenia, the inventors conclude that the improvement in negative thinking will additionally contribute to an overall improvement of Schizophrenia.

An improvement of Schizophrenia in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of Schizophrenia in a patient also suffering from associated negative thinking, as reflected by a reduction in the CGI-S score, occurs not later than about 2 hours after the last administration of 5-MeO-DMT or a pharmaceutically acceptable salt thereof. An improvement of Schizophrenia in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Compared to other psychedelics, like LSD, psylocibin or DMT, 5-MeO-DMT or a pharmaceutical acceptable salt thereof can be administered to patients suffering from Schizophrenia associated with negative thinking, preferably using dosing schemes as described herein, without a significant risk of inducing mania or hypomania.

Preferably, the patient suffering from Schizophrenia associated with negative thinking does not experience treatment-emergent mania or hypomania.

### Dementia

Dementia is generally characterized by a loss of memory, language, problem-solving and other thinking abilities that are severe enough to interfere with daily life.

Dementia is caused by brain changes which trigger a decline in cognitive abilities and also affect behaviour, feelings and relationships.

Negative thinking or at least aspects thereof may be assessed, for instance, by using the Positive and Negative Affect Schedule-Expanded Form (PANAS-X).

Dementia affects various functional and structural connectivity networks in the brain, as can be shown by magnetic resonance imaging studies. Alterations within and/or between the default mode network (DMN), the salience network, and the central executive network (CEN) are observed.

Treating a patient suffering from Dementia and associated negative thinking with 5-MeO-DMT or a pharmaceutically acceptable salt thereof reduces or eliminates the negative thinking and leads to an improvement of Dementia.

The reduction or elimination of negative thinking in a patient suffering from Dementia is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Dementia occurs not later than about 2 hours after the last administration of 5-MeO-DMT or a pharmaceutically acceptable salt thereof. The reduction or elimination of negative thinking in a patient suffering from Dementia preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from Dementia, is reflected by at least an improvement in the score of one or more of the basic negative emotion scales of the Positive and Negative Affect Schedule-Expanded Form (PANAS-X), about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from Dementia, as reflected by at least an improvement in the score of one or more of the basic negative emotion scales of the Positive and Negative Affect Schedule-Expanded Form (PANAS-X), occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from Dementia, as reflected by at least an improvement in the score of one or more of the basic negative emotion scales of the Positive and Negative Affect Schedule-Expanded Form (PANAS-X), preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

As indicated above, negative thinking occurs in patients suffering from Dementia. An improvement in negative thinking will therefore also lead to an improvement of the Dementia. Since negative thinking furthermore also affects other aspects of Dementia, the inventors conclude that the improvement in negative thinking will additionally contribute to an overall improvement of the Dementia.

An improvement of the Dementia in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the Dementia in a patient also suffering from associated negative thinking, as reflected by a reduction in the CGI-S score, occurs not later than about 2 hours after the last administration of 5-MeO-DMT or a pharmaceutically acceptable salt thereof. An improvement of the Dementia in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Alzheimer's Dementia (AD) is a highly prevalent neurodegenerative disorder with an insidious onset and gradual progression of cognitive deficits.

Deficits in emotional processing are a consistent feature of AD.

Repetitive negative thinking (RNT) has been shown to be linked to cognitive decline profiles similar to early AD as well as neuroimaging evidence of AD biomarkers. Thus, RNT is an important marker of dementia risk.

Negative thinking or at least aspects thereof may be assessed, for instance, by using the Positive and Negative Affect Schedule-Expanded Form (PANAS-X).

AD affects various functional and structural connectivity networks in the brain which are associated with the topography, clinical phenotype, and severity of the disease. Magnetic resonance imaging studies have demonstrated successive structural and functional disconnection among brain regions supporting the idea that AD is a disconnection syndrome. Alterations within and/or between the default mode network (DMN), salience network, and central executive network (CEN) are observed both in patients with AD and individuals who were at high risk for developing AD.

Therefore, influencing those networks by a therapy according to the invention will lead to an improvement of the negative thinking, and also to an improvement of the symptoms of AD.

Treating a patient suffering from AD and associated negative thinking with 5-MeO-DMT or a pharmaceutically acceptable salt thereof reduces or eliminates the negative thinking and leads to an improvement of AD.

The reduction or elimination of negative thinking in a patient suffering from AD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from AD occurs not later than about 2 hours after the last administration of 5-MeO-DMT or a pharmaceutically acceptable salt thereof. The reduction or elimination of negative thinking in a patient suffering from AD preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from AD, is reflected by at least an improvement in the score of one or more of the basic negative emotion scales of the Positive and Negative Affect Schedule-Expanded Form (PANAS-X), about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from AD, as reflected by at least an improvement in the score of one or more of the basic negative emotion scales of the Positive and Negative Affect Schedule-Expanded Form (PANAS-X), occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from AD, as reflected by at least an improvement in the score of one or more of the basic negative emotion scales of the Positive and Negative Affect Schedule-Expanded Form (PANAS-X), preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

As indicated above, negative thinking is an important aspect in patients suffering from AD. An improvement in negative thinking will therefore also lead to an improvement of the AD. Since negative thinking furthermore also affects other aspects of AD, the inventors conclude that the improvement in negative thinking will additionally contribute to an overall improvement of the AD.

An improvement of the AD in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the AD in a patient also suffering from associated negative thinking, as reflected by a reduction in the CGI-S score, occurs not later than about 2 hours after the last administration of 5-MeO-DMT or a pharmaceutically acceptable salt thereof. An improvement of the AD in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

### Eating Disorders

An Eating Disorder is a mental disorder characterised by abnormal eating behaviours that negatively affect a person's physical or mental health.

A patient suffering from an Eating Disorder may suffer from a treatment resistant form of the disorder.

Difficulties in identifying and regulating negative emotions are noted as a key feature of anorexia nervosa. In anorexia nervosa sadness, anger, disgust and fear have been found to be key negative emotions that are linked to maladaptive regulation strategies such as avoidance and suppression which drive specific Eating Disorder behaviours such as restrictive eating and purging.

Sadness was perceived as toxic and shaming, prompting inhibition by patients with anorexia nervosa.

In the context of eating disorders, shame-proneness rather than guilt-proneness was associated with eating disorder behaviours such as self-induced vomiting and binge eating.

In patients suffering from Eating Disorders, results from functional network connectivity studies indicate disrupted resting-state connectivity within and/or between executive networks, the default mode network and the salience network.

The relevance of negative thinking in patients suffering from Eating Disorders is reflected in, for example, the Clinical Impairment Assessment Questionnaire (CIA3.0), which is a 16-item, self-report instrument, containing an item relating to feelings of guilt.

Negative thinking or at least aspects thereof may be assessed, for instance, by using the Positive and Negative Affect Schedule-Expanded Form (PANAS-X).

Treating a patient suffering from an Eating Disorder, including a treatment resistant form of the disorder, and associated negative thinking with 5-MeO-DMT or a pharmaceutically acceptable salt thereof reduces or eliminates the negative thinking and leads to an improvement of the Eating Disorder.

The reduction or elimination of negative thinking in a patient suffering from an Eating Disorder is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from an Eating Disorder occurs not later than about 2 hours after the last administration of 5-MeO-DMT or a pharmaceutically acceptable salt thereof. The reduction or elimination of negative thinking in a patient suffering from an Eating Disorder preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from an Eating Disorder, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from an Eating Disorder, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from an Eating Disorder, as reflected by at least an improvement in the score of one or more of the basic negative emotion scales of the Positive and Negative Affect Schedule-Expanded Form (PANAS-X), preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

As indicated above, negative thinking is an important aspect in patients suffering from an Eating Disorder. An improvement in negative thinking will therefore also lead to an improvement of the Eating Disorder. Since negative thinking furthermore also affects other aspects of an Eating Disorder, the inventors conclude that the improvement in negative thinking will additionally contribute to an overall improvement of the Eating Disorder.

An improvement of the Eating Disorder in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the Eating Disorder in a patient also suffering from associated negative thinking, as reflected by a reduction in the CGI-S score, occurs not later than about 2 hours after the last administration of 5-MeO-DMT or a pharmaceutically acceptable salt thereof. An improvement of the Eating Disorder in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

### Attention Deficit Hyperactivity Disorder (ADHD)

Attention Deficit Hyperactivity Disorder (ADHD) is a condition that affects a patient's behaviour. A patient suffering from ADHD can seem restless, may have trouble concentrating and may act on impulse.

Patients with ADHD may also have additional problems, such as sleep and anxiety disorders.

A patient suffering from ADHD may suffer from a treatment resistant form of the disorder.

ADHD is associated with emotional regulation difficulties, particularly around the implementation stage of regulation strategies. Comorbid eating disorders are common with ADHD. Negative emotions in ADHD are found to mediate the relationship between the condition and disordered eating.

A study that compared boys with ADHD to boys without ADHD found that behavioural manifestations of sadness and guilt were exhibited more frequently in the ADHD group compared to the control group.

Negative thinking or at least aspects thereof may be assessed, for instance, by using the State Shame and Guilt Scale (SSGS) or the Positive and Negative Affect Schedule-Expanded Form (PANAS-X).

Resting state network analysis in patients suffering from ADHD reveals dysfunctional connectivity across multiple brain resting state networks, in particular dysfunctional connectivity within and/or between distinct regions of the default mode network, the dorsal attention network and the salience network.

Treating a patient suffering from ADHD, including a treatment resistant form of the disorder, and associated negative thinking with 5-MeO-DMT or a pharmaceutically acceptable salt thereof reduces or eliminates the negative thinking and leads to an improvement of the ADHD.

The reduction or elimination of negative thinking in a patient suffering from ADHD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from ADHD occurs not later than about 2 hours after the last administration of 5-MeO-DMT or a pharmaceutically acceptable salt thereof. The reduction or elimination of negative thinking in a patient suffering from ADHD preferably persists until at least 6 days; in particular until at least 14 days; more preferably until at least 28 days after the last administration of 5-MeO-DMT or a pharmaceutically acceptable salt thereof.

A reduction or elimination of negative thinking in a patient suffering from ADHD, in particular of guilt feelings, is reflected by at least an improvement in the score of the guilt subscale of the State Shame and Guilt Scale (SSGS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

A reduction or elimination of negative thinking in a patient suffering from ADHD, in particular of guilt feelings, as reflected by at least an improvement in the score of the guilt subscale of the State Shame and Guilt Scale (SSGS), occurs not later than about 2 hours after the last administration of 5-MeO-DMT or a pharmaceutically acceptable salt thereof.

The reduction or elimination of negative thinking in a patient suffering from ADHD, in particular of guilt feelings, as reflected by at least an improvement in the score of the guilt subscale of the State Shame and Guilt Scale (SSGS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from ADHD, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from ADHD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from ADHD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

As indicated above, negative thinking is an important aspect in patients suffering from ADHD. An improvement in negative thinking will therefore also lead to an improvement of the ADHD. Since negative thinking furthermore also affects other aspects of ADHD, the inventors conclude that the improvement in negative thinking will additionally contribute to an overall improvement of the ADHD.

An improvement of the ADHD in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the ADHD in a patient also suffering from associated negative thinking, as reflected by a reduction in the CGI-S score, occurs not later than about 2 hours after the last administration of 5-MeO-DMT or a pharmaceutically acceptable salt thereof.

An improvement of the ADHD in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

### Personality Disorders

Schizotypal Personality Disorder is a mental health condition marked by a consistent pattern of intense discomfort with relationships and social interactions. Patients suffering from Schizotypal Personality Disorder have unusual thoughts, speech and behaviours, which hinder their ability to form and maintain relationships.

A patient suffering from Schizotypal Personality Disorder may suffer from a treatment resistant form of the disorder.

Negative thinking or at least aspects thereof may be assessed, for instance, by using the Positive and Negative Affect Schedule-Expanded Form (PANAS-X).

In patients with Schizotypal Personality Disorder, DMN functional connectivity, particularly that involving cognitive or emotional regulation, is altered. Neuroimaging analysis by fMRI further reveals alterations within and/or between frontoparietal network and dorsal attention network.

Treating a patient suffering from Schizotypal Personality Disorder, including a treatment resistant form of the disorder, and associated negative thinking with 5-MeO-DMT or a pharmaceutically acceptable salt thereof reduces or eliminates the negative thinking and leads to an improvement of Schizotypal Personality Disorder.

The reduction or elimination of negative thinking in a patient suffering from Schizotypal Personality Disorder is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from Schizotypal Personality Disorder occurs not later than about 2 hours after the last administration of 5-MeO-DMT or a pharmaceutically acceptable salt thereof. The reduction or elimination of negative thinking in a patient suffering from Schizotypal Personality Disorder preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from Schizotypal Personality Disorder, is reflected by at least an improvement in the score of one or more of the basic negative emotion scales of the Positive and Negative Affect Schedule-Expanded Form (PANAS-X), about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from Schizotypal Personality Disorder, as reflected by at least an improvement in the score of one or more of the basic negative emotion scales of the Positive and Negative Affect Schedule-Expanded Form (PANAS-X), occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking in a patient suffering from Schizotypal Personality Disorder, as reflected by at least an improvement in the score of one or more of the basic negative emotion scales of the Positive and Negative Affect Schedule-Expanded Form (PANAS-X), preferably persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

As indicated above, negative thinking occurs in patients suffering from Schizotypal Personality Disorder. An improvement in negative thinking will therefore also lead to an improvement of the Schizotypal Personality Disorder. Since negative thinking furthermore also affects other aspects of Schizotypal Personality Disorder, the inventors conclude that the improvement in negative thinking will additionally contribute to an overall improvement of the Schizotypal Personality Disorder.

An improvement of the Schizotypal Personality Disorder in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the Schizotypal Personality Disorder in a patient also suffering from associated negative thinking, as reflected by a reduction in the CGI-S score, occurs not later than about 2 hours after the last administration of 5-MeO-DMT or a pharmaceutically acceptable salt thereof. An improvement of the Schizotypal Personality Disorder in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

Compared to other psychedelics, like LSD, psylocibin or DMT, 5-MeO-DMT or a pharmaceutical acceptable salt thereof can be administered to patients suffering from Schizotypal Personality Disorder associated with negative thinking, preferably using dosing schemes as described herein, without a significant risk of inducing mania or hypomania.

Preferably, the patient suffering from Schizotypal Personality Disorder associated with negative thinking does not experience treatment-emergent mania or hypomania.

### Borderline Personality Disorder (BPD)

Persistent mood instability, impulsivity, identity disturbance and interpersonal dysfunction are characteristic traits for Borderline Personality Disorder (BPD).

A patient suffering from BPD may suffer from a treatment resistant form of the disorder.

There is a significant relationship between negative affect and BPD. Students with greater BPD features experience higher daily negative affect compared to healthy controls.

The Borderline Symptom List 95 (BSL-95) contains several items relevant for negative thinking, such as helplessness, hopelessness, worthlessness and guilt, indicating that negative thinking is an integral aspect of BPD.

Negative thinking or at least aspects thereof may be assessed, for instance, by using the State Shame and Guilt Scale (SSGS), the Positive and Negative Affect Schedule-Expanded Form (PANAS-X) or the State Hope Scale (SHS).

Patients with BPD show abnormal connectivity patterns in resting-state networks. Analysis of functional connectivity of brain resting state networks using functional magnetic resonance imaging reveals alterations within and/or between various networks, such as for example the default mode network and salience network.

Treating a patient suffering from BPD, including a treatment resistant form of the disorder, and associated negative thinking with 5-MeO-DMT or a pharmaceutically acceptable salt thereof reduces or eliminates the negative thinking and leads to an improvement of the BPD.

The reduction or elimination of negative thinking in a patient suffering from BPD is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from BPD occurs not later than about 2 hours after the last administration of 5-MeO-DMT or a pharmaceutically acceptable salt thereof. The reduction or elimination of negative thinking in a patient suffering from BPD preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from BPD, is reflected by at least an improvement in the score of the guilt subscale of the State Shame and Guilt Scale (SSGS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from BPD, as reflected by at least an improvement in the score of the guilt subscale of the State Shame and Guilt Scale (SSGS), occurs not later than about 2 hours after the last administration of 5-MeO-DMT or a pharmaceutically acceptable salt thereof. The reduction or elimination of negative thinking in a patient suffering from BPD, as reflected by at least an improvement in the score of the guilt subscale of the State Shame and Guilt Scale (SSGS), preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking in a patient suffering from BPD, is reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from BPD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking preferably in a patient suffering from BPD, as reflected by at least an improvement in the score of one or more of the basic negative emotion scales of the Positive and Negative Affect Schedule-Expanded Form (PANAS-X), in particular in the score of the guilt scale, wherein the recall period does not start earlier than the time point when acute psychedelic experiences have subsided after the last administration to the assessment time point persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

Alternatively or additionally, the reduction or elimination of negative thinking in a patient suffering from BPD, in particular of hopelessness, is reflected by at least an improvement in the score of the State Hope Scale (SHS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from BPD, in particular of hopelessness, as reflected by at least an improvement in the score of the State Hope Scale (SHS), occurs not later than about 2 hours after the last administration of 5-MeO-DMT or a pharmaceutically acceptable salt thereof. The reduction or elimination of negative thinking in a patient suffering from BPD, in particular of hopelessness, as reflected by at least an improvement in the score of the State Hope Scale (SHS), preferably persists until at least 6 days; in particular until at least 14 days; more preferably until at least 28 days after the last administration of 5-MeO-DMT or a pharmaceutically acceptable salt thereof.

As indicated above, negative thinking is an integral aspect in patients suffering from BPD. An improvement in negative thinking will therefore also lead to an improvement of the BPD. Since negative thinking furthermore also affects other aspects of BPD, the inventors conclude that the improvement in negative thinking will additionally contribute to an overall improvement of the BPD.

An improvement of the BPD in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the BPD in a patient also suffering from associated negative thinking, as reflected by a reduction in the CGI-S score, occurs not later than about 2 hours after the last administration of 5-MeO-DMT or a pharmaceutically acceptable salt thereof. An improvement of the BPD in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

### Medical Health Conditions Leading to an Associated Mental or Nervous System Condition

The negative thinking may occur in a patient suffering from medical health condition leading to an associated mental or nervous system condition.

### Negative Thinking Due to Traumatic Brain Injury (TBI)

A traumatic brain injury (TBI) is an injury to the brain caused by an external force.

Traumatic brain injuries are characterized as primary or secondary brain injuries. A primary brain injury refers to the structural damage created during the time of impact from contact, acceleration-deceleration, and/or rotational forces. A secondary brain injury refers to the damage sustained from the subsequent cellular processes that occur from the primary injury (ie, hypoxia and/or raised intracranial pressure).

TBI can have wide-ranging physical and psychological effects, which may appear immediately after the traumatic event, while others may appear days or weeks later. The cognitive, behavioural, and sensorimotor disabilities that result from TBI often dramatically decrease quality of life. TBI can cause irritability, anxiety, insomnia, and depression. Depression after a TBI is characterized by persistent sadness, feelings of worthlessness and hopelessness.

Negative thinking or at least aspects thereof may be assessed, for instance, by using the Positive and Negative Affect Schedule-Expanded Form (PANAS-X) or the State Hope Scale (SHS).

Resting state functional connectivity analysis reveals alterations in overall functional connectivity within and/or between resting state networks, such as the DMN, the frontoparietal network, the executive network, and the sensory motor network. For instance, highly connected regions that can be found within the DMN are particularly susceptible to alterations in functional connectivity following traumatic brain injury.

Treating a patient suffering from TBI and associated negative thinking with 5-MeO-DMT or a pharmaceutically acceptable salt thereof reduces or eliminates the negative thinking and leads to an improvement of the BPD.

The reduction or elimination of negative thinking in a patient suffering from TBI is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from TBI occurs not later than about 2 hours after the last administration of 5-MeO-DMT or a pharmaceutically acceptable salt thereof. The reduction or elimination of negative thinking in a patient suffering from TBI preferably persists until at least 6 days; in particular until at least 14 days; more preferably until at least 28 days after the last administration of 5-MeO-DMT or a pharmaceutically acceptable salt thereof.

The reduction or elimination of negative thinking in a patient suffering from TBI, is reflected by at least an improvement in the score of one or more of the basic negative emotion scales of the Positive and Negative Affect Schedule-Expanded Form (PANAS-X) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof, wherein the recall period does not start earlier than the time point when acute psychedelic experiences have subsided after the last administration to the assessment time point.

The reduction or elimination of negative thinking in a patient suffering from TBI, as reflected by at least an improvement in the score of one or more of the basic negative emotion scales of the Positive and Negative Affect Schedule-Expanded Form (PANAS-X), occurs not later than about 2 hours after the last administration of 5-MeO-DMT or a pharmaceutically acceptable salt thereof, wherein the recall period does not start earlier than the time point when acute psychedelic experiences have subsided after the last administration to the assessment time point. The reduction or elimination of negative thinking preferably in a patient suffering from TBI, as reflected by at least an improvement in the score of one or more of the basic negative emotion scales of the Positive and Negative Affect Schedule-Expanded Form (PANAS-X), persists until at least 6 days; in particular until at least 14 days; more preferably until at least 28 days after the last administration of 5-MeO-DMT or a pharmaceutically acceptable salt thereof, wherein the recall period does not start earlier than the time point when acute psychedelic experiences have subsided after the last administration to the assessment time point.

Alternatively or additionally, the reduction or elimination of negative thinking in a patient suffering from TBI, in particular of hopelessness, is reflected by at least an improvement in the score of the State Hope Scale (SHS) about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

The reduction or elimination of negative thinking in a patient suffering from TBI, in particular of hopelessness, as reflected by at least an improvement in the score of the State Hope Scale (SHS), occurs not later than about 2 hours after the last administration of 5-MeO-DMT or a pharmaceutically acceptable salt thereof. The reduction or elimination of negative thinking in a patient suffering from TBI, in particular of hopelessness, as reflected by at least an improvement in the score of the State Hope Scale (SHS), preferably persists until at least 6 days; in particular until at least 14 days; more preferably until at least 28 days after the last administration of 5-MeO-DMT or a pharmaceutically acceptable salt thereof.

Negative thinking due to TBI is related to other symptoms which develop as a consequence of TBI. An improvement in negative thinking will also lead to an improvement of such other symptoms.

An improvement of the BPD in a patient also suffering from associated negative thinking, as reflected by a reduction in the CGI-S score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

An improvement of the BPD in a patient also suffering from associated negative thinking, as reflected by a reduction in the CGI-S score, occurs not later than about 2 hours after the last administration of 5-MeO-DMT or a pharmaceutically acceptable salt thereof. An improvement of the BPD in a patient also suffering from associated negative thinking, as reflected by a reduction in the CGI-S score, preferably persists until at least 6 days; in particular until at least 14 days; more preferably until at least 28 days after the last administration of 5-MeO-DMT or a pharmaceutically acceptable salt thereof.

### EXAMPLES

The following Examples are listed to aid understanding of the invention and are not intended and should not be construed to limit in any way the invention set forth in the claims which follow thereafter.

### Example 1—5-MeO-DMT Aerosol Generation and Administration

Step 1: A stock solution of 5-MeO-DMT free base in 100% ethanol is prepared in a volumetric flask, so that the target dosage of 5-MeO-DMT free base to be administered via inhalation to the volunteer or patient is contained in a solution volume of 200 μl. Typical target dosages are from 1 mg to 25 mg 5-MeO-DMT. E.g. for a target dosage of 18 mg 5-MeO-DMT, 90 mg of 5-MeO-DMT will be dissolved in 100% ethanol for a final solution volume of 1 ml. Aliquots of the stock solution can then be stored in vials until further use.

Step 2: 200 μl of the solution is transferred to a dosing capsule containing the drip pad (Storz & Bickel, Germany), and then the dosing capsule is closed with its lid.

Step 3: The dosing capsule filled with the 5-MeO-DMT ethanol solution is transferred to the filling chamber of a first Volcano Medic Vaporizer, which has been pre-heated with the temperature set at 55° C. Then the airflow of the vaporizer is switched on for 60 seconds at the pre-set rate of about 12 l/min. The heated air will flow through the dosing capsule, allowing the ethanol to evaporate, with the target dosage of 5-MeO-DMT being left in the capsule, as a thin layer covering the stainless-steel wire mesh. Accurate preparation of the dosing capsule can be confirmed by demonstrating that the final weight increase of the capsule compared to the weight of the empty capsule is about equal to the target dosage of 5-MeO-DMT.

Step 4: The prepared dosing capsule is removed from the filling chamber. It is then transferred to the filling chamber of a second Volcano Medic Vaporizer, which has been pre-heated with the temperature set at 210° C. and the airflow on for at least 5 minutes and then turned off immediately prior to transfer. An inhalation balloon with a valve (Storz & Bickel, Germany) is mounted on the socket of the filling chamber, the filling chamber is closed tightly and immediately afterwards the airflow is switched on for exactly 15 seconds at the pre-set flow rate of about 12 l/min, and then turned off. This will allow the full dose of 5-MeO-DMT to aerosolize and be distributed in approximately 3 liters of air in the inhalation balloon. Accurate aerosolization of the 5-MeO-DMT can be confirmed by demonstrating that the capsule weight has returned to about its initial weight.

Step 5: The balloon is then disconnected from the filling chamber, which automatically closes the valve. After attachment of the mouthpiece to the balloon, the aerosol is ready for immediate administration to the volunteer or patient.

Step 6: To prepare for the administration, the patient is asked to initially perform 1-2 deep inhalations with full exhalations, ending this sequence with a deep exhalation. Then, with the mouthpiece firmly held against the lips, the full and complete volume of the inhalation balloon is inhaled in one inhalation, holding the breath for 10 (±2.5) seconds, followed by a normal exhalation. After completing the inhalation procedure, the patient will be instructed to lie down.

Further details regarding the administration of 5-MeO-DMT by inhalation are disclosed in Example 1 of WO 2020/169850 A1, the contents of which is incorporated herein by reference.

### Example 2—Preparation of 5-MeO-DMT in High Purity

5-MeO-DMT (2.0 g) was dissolved in MTBE (4 mL, 2.0 volumes) at 35 to 40° C. before being cooled to room temperature over 30 minutes. After stirring at room temperature for 50 minutes no crystallisation was observed, therefore, the batch temperature was decreased to 7 to 12° C. over 30 minutes. After stirring at 7 to 12° C. for 10 minutes crystallisation occurred. The batch was subsequently filtered following a 1 hour stir out at 7 to 12° C. After washing with MTBE (1 mL, 0.5 volumes), at 7 to 12° C., the batch was pulled dry under vacuum for 3.5 hours to yield a pale orange solid in 1.02 g (50% recovery). The isolated solid was analysed for purity by HPLC as described in WO 2020/169850 A1. The purity was found to be 99.74% area.

The results from the analysis further indicate that the level of individual impurities was below 0.10% area. Solvent analysis of sample indicated an MTBE level of 17 ppm.

### Example 3—Preparation of 5-MeO-DMT Hydrobromide Salt

5-MeO-DMT HBr was prepared on a 100 mg scale.

5-MeO-DMT free base was combined with isopropyl acetate (10 vols), and the resulting solution of 5-MeO-DMT was heated to 50° C. HBr was charged (1 M in ethanol, 1 eq) in one single aliquot. The mixture was held at temperature and equilibrated for 3 hours.

After 1 hour, a suspension had formed. The suspension was finally cooled to room temperature and equilibrated for 18 hours. Solids were isolated by filtration and dried in vacuo at 40° C. for 18 hours.

An off-white crystalline material was obtained.

The salt has a melting point of 174° C. and is characterized by an X-ray diffraction pattern comprising peaks at 14.5°2⋅±0.2°2⋅; 16.7°2⋅±0.2°2⋅; 17.0°2⋅±0.2°2⋅; 20.6°2⋅±0.2°2⋅; 20.7° 2±0.2° 2⋅; 21.4° 2±0.2° 2⋅; 24.2° 2±0.2° 2⋅; 24.8° 2 0.2° 2; 25.3° 2±0.2° 2⋅; 27.4°2⋅±0.2°2⋅; measured using Cu K⋅ radiation.

### Example 4—Determination of Inhibition Constants for Central 5-HT1A and 5-HT2A Receptors in Post-Mortem Human Brain Membrane Preparations

In this study, the affinity of three psychedelic test compounds (psilocin, DMT and 5-MeO-DMT) for 5-HT1A and 5-HT2A receptors in post-mortem human brain tissue from the hippocampus and frontal cortex, respectively, was determined using the technique of radioligand binding.

Human brain samples were obtained from the Edinburgh Sudden Death Brain Bank. All donors were sudden deaths with no prior history of coma, psychiatric or neurological disorders and under the age of 65 with a post-mortem interval of less than or equal to 72 hours.

Binding to 5-HT1A receptors in post-mortem human hippocampus

Hippocampus was homogenised in ice-cold 0.25 M sucrose (1:30 w/v) using a motor driven Teflon pestle (12 strokes at 120 rpm). Myelin and cell debris were removed by centrifugation at 1,000 g for 10 minutes. The supernatant was stored on ice and the pellet re-homogenised in 0.25 M sucrose (1:15 w/v) and centrifuged at 750 g for 10 minutes.

The supernatants were combined and diluted in ice-cold membrane preparation buffer, (1:100 w/v) using a tight-fitting glass/Teflon homogeniser (12 strokes, 800 rpm) and centrifuged at 20,500 g for 10 minutes. The pellet was resuspended in ice-cold membrane preparation buffer and incubated at 37° C. for 10 minutes before being centrifuged at 20,500 g for 10 minutes. The pellet was resuspended and centrifuged a final time to wash the tissue (20,500×g for 10 mins). The resulting pellet was then resuspended in ice-cold assay buffer at a tissue concentration equivalent to 3.125 mg wet weight of tissue/ml. All centrifugations were carried out at 4° C. The membrane preparation buffer consisted of 50 mM Tris-HCl, pH 7.7, 4 mM CaCl₂ and 0.1% ascorbic acid. The assay buffer consisted of 50 mM Tris, pH 7.7, 4 mM CaCl₂, 0.1% ascorbic acid and 10 μM Pargyline.

For saturation binding analysis, hippocampal membranes (400 μl; equivalent 1.25 mg wet weight tissue/tube) was incubated with 50 μl of 0.075-9.6 nM [³H]8-OH-DPAT and either 50 μl of assay buffer (total binding) or 50 μl of 1 μM WAY 100635 (non-specific binding) at 25° C. for 30 minutes. The wash buffer consisted of 50 mM Tris, pH 7.7.

In a displacement assay, hippocampal membranes (400 μl; equivalent 1.25 mg wet weight tissue/tube) were incubated with 50 μl of 0.6 nM [³H]8-OH-DPAT and either 50 μl of assay buffer (total binding) or 50 μl of 1·M WAY 100635 (non-specific binding) or 50 μl of one of the test compounds in one of ten concentrations between 1 and 10000 nM at 25° C. for 30 minutes.

Membrane bound radioactivity was recovered by filtration under vacuum through Skatron 11731 filters, pre-soaked in 0.5% polyethylenimine (PEI) using a Skatron cell harvester. Filters were rapidly washed with ice-cold wash buffer (wash setting 0,9,9) and radioactivity determined by liquid scintillation counting (1 ml Packard MV Gold scintillator).

The concentration of compound required to inhibit 50% of specific binding (IC₅₀) and the Hill Slope were calculated by using non-linear regression. The Kᵢ was calculated using the one-site binding model allowing for ligand depletion.

### Binding to 5-HT2A Receptors in Post-Mortem Human Frontal Cortex

Frontal cortex was homogenised in ice-cold 0.25 M sucrose (1:30 w/v) using a motor driven Teflon pestle (12 strokes at 120 rpm). Myelin and cell debris was removed by centrifugation at 1,000 g for 10 minutes. The supernatant was stored on ice and the pellet re-homogenised in 0.25 M sucrose (1:15 w/v) and centrifuged at 750 g for 10 minutes. The supernatants were combined and diluted in ice-cold 50 mM Tris-HCl assay buffer, pH 7.4 (1:100 w/v), homogenised using a tight-fitting glass/Teflon homogeniser (12 strokes, 800 rpm) and centrifuged at 20,500 g for 10 minutes. The pellet was centrifuged a further two times to wash the tissue (20,500×g for 10 mins). The resulting pellet was then resuspended in ice-cold 50 mM Tris-HCl assay buffer, pH 7.4 at a tissue concentration equivalent to 10 mg wet weight of tissue/ml. All centrifugations were carried out at 4° C.

For saturation binding analysis, frontal cortical membranes (400 μl; equivalent to 4 mg wet weight of tissue/tube) were incubated with 50 μl of 0.00625-0.8 nM [³H]MDL-100,907 and either 50 μl of assay buffer or 50 μl of 10·M ketanserin (non-specific binding) at 25° C. for 60 minutes. The assay and wash buffer consisted of 50 mM Tris-HCl buffer pH 7.4.

In a displacement assay, frontal cortical membranes (400 μl; equivalent 4 mg wet weight tissue/tube) was incubated with 50 μl of 0.1 nM [3H]MDL-100,907 and either 50 μl of assay buffer (total binding) or 50 μl of 10·M ketanserin (non-specific binding) or 50 μl of one of the test compounds in one of ten concentrations between 1 and 10000 nM at 25° C. for 60 minutes.

Membrane bound radioactivity was recovered and determined as above. Data analysis was also as above.

### Results

The dissociation constant (K_{d} value) of [³H]8-OH-DPAT for 5-HT1A receptors in hippocampal membranes from post-mortem human brain tissue was determined for each of the three donors. The dissociation constants (K_{d} values) obtained were 0.51, 0.28 and 0.52 nM, respectively.

Mean inhibition constants (Kᵢ values) for psilocin, DMT and 5-MeO-DMT were 48, 38 and 1.80 nM (mean n=3), respectively. All compounds gave Hill slopes approximating to unity, suggesting a one-site binding model.

The dissociation constant (K_{d} values) of [³H]MDL-100,907 for 5-HT2A receptors in frontal cortical membranes from post-mortem human brain tissue was determined for each of the three donors. The dissociation constants (K_{d} values) obtained were 0.11, 0.08 and 0.08 nM, respectively.

Mean inhibition constants (Kᵢ values) for psilocin, DMT and 5-MeO-DMT were 37, 117 and 122 nM (mean n=3), respectively. All compounds gave Hill slopes approximating to unity, suggesting a one-site binding model.

The selectivity ratio of psilocin, DMT and 5-MeO-DMT for 5-HT2A over 5-HT1A receptors was 0.78, 3.1 and 68, respectively.

### Example 5—Toxicological Testing of 5-MeO-DMT

5-MeO-DMT did not induce mutation in four histidine-requiring bacterial strains (TA98, TA100, TA1535 and TA1537) of Salmonella typhimurium, and one tryptophan-requiring strain (WP2 uvrA pKM101) of Escherichia coli. These conditions included treatments at concentrations up to 5000 μg/plate (the maximum recommended concentration according to current regulatory guidelines), in the absence and in the presence of a rat liver metabolic activation system (S-9).

### Example 6—Assessment of the Pharmacokinetics of 5-MeO-DMT and Bufotenine

In order to investigate the pharmacokinetic properties of 5-MeO-DMT, three groups of 8 subjects each were formed. Subjects were administered a single dose of 6 mg; 12 mg or 18 mg 5-MeO-DMT via inhalation. Blood samples were obtained at 1; 2; 4; 7; 10; 15; 20; 30; 45 min and 1; 1.5; 2; 3; 4 hours after administration.

5-MeO-DMT concentrations were determined using LC-MS/MS. PK parameters were generated by algebraic analysis of the concentration versus time plots for each individual. The analysis was carried out using the software Phoenix WinNonlin 6.3.

Median Cmax values obtained for the three groups were 11.85 ng/ml (6 mg group), 22.90 ng/ml (12 mg group) and 38.45 ng/ml (18 mg group).

Table 1 below shows median percentage plasma concentrations relative to Cmax as determined for the time points indicated.

| | Median percentage plasma concentration relative to Cmax | | | | | | | | | | | | | |
|---|---|---|---|---|---|---|---|---|---|---|---|---|---|---|
| time (min) | 1 | 2 | 4 | 7 | 10 | 15 | 20 | 30 | 45 | 60 | 90 | 120 | 180 | 240 |
| 6 mg | 96 | 98 | 83 | 56 | 40 | 22 | 16 | 10 | 8 | 4 | 2 | 1 | 0 | N.A. |
| 12 mg | 100 | 79 | 59 | 30 | 22 | 12 | 8 | 5 | 3 | 2 | 0 | 0 | 0 | 0 |
| 18 mg | 100 | 79 | 59 | 30 | 22 | 12 | 8 | 5 | 3 | 2 | 0 | 0 | 0 | 0 |

Pharmacokinetic measurements were also carried out for dosing schemes relying on uptitration. Substantially similar results were obtained.

Blood concentrations were also determined for the 5-MeO-DMT metabolite bufotenine.

Only in few samples, concentrations were above the lower level of quantification (LLOQ) (25 μg/ml). From 15 min onwards, the bufotenine concentration was always below the LLOQ.

Substantially similar observations were made when subjects receiving an uptitration scheme were included.

### Example 7—Clinical trial in patients suffering from TRD

A Phase 1/2 clinical trial of 5-MeO-DMT, administered via inhalation as described herein, in patients with treatment-resistant major depressive disorder (TRD) has been completed. This trial was designed in two parts. Part A was an open-label, single-arm, single-dose Phase 1 trial with two dose levels (12 mg (n=4) and 18 mg (n=4)). Part B was an open-label, single-arm Phase 2 trial applying an individualized dosing regimen with intra-patient dose escalation with 5-MeO-DMT. Patients (n=8) received at least one and up to three doses of 5-MeO-DMT (6 mg, 12 mg and 18 mg) in a single day, with higher doses only being administered if a peak experience was not achieved at the previously administered dose. The primary endpoint of Part A was to assess the safety and tolerability of single dosing of 5-MeO-DMT in patients with TRD. The primary endpoint of Part B was to assess the effects on the severity of depression, as assessed by the proportion of patients in remission on day seven after dosing, defined as a MADRS total score of less than or equal to 10.

In Part A, 3 of 4 patients in both groups (12 mg and 18 mg) experienced at least one ADR, all of which were mild and resolved spontaneously. No SAEs were reported.

Two of four patients (50%) in the 12 mg group and one of four patients (25%) in the 18 mg group had a MADRS remission on day seven after dosing, and one further patient (25%) in the 18 mg group had a MADRS clinical response on day seven after dosing. The mean MADRS change from baseline at day seven was −21.0 (−65%) in the 12 mg group and −12.8 (−41%) in the 18 mg group.

In Part B, 7 of 8 patients (87.5%) experienced at least one ADR. All ADRs resolved spontaneously. No SAEs were reported.

The primary endpoint was met with seven of eight patients (87.5%) achieving a MADRS remission on day seven (p<0.0001). The mean MADRS change from baseline at day seven was 24.4 (76%).

No clinically significant changes were observed in either Part A or Part B in any of the safety laboratory analyses, vital signs, psychiatric safety assessments or measures of cognitive function.

Results are summarized in the tables below.

TABLE 2
A Scores recorded against relevant MADRS and BPRS items for patients
as-signed to intra-day individualised dosing regimen (IDR). The
item scores represent the sum of the individual patient scores
for all patients (n = 8) in the IDR group. Assessment 2 hours
(MADRS) or 3 hours (BPRS) after administration of the last dose.

| | | | | Improve- | Percent- |
|---|---|---|---|---|---|
| | | Base- | | ment | age |
| | | line | 2/3 | 2/3 | improve- |
| Scale item | Scale | IDR | hours | hours | ment |
| Concentration | MADRS | 30 | 11 | 19 | 63.33% |
| Difficulties | | | | | |
| Lassitude | MADRS | 27 | 10 | 17 | 62.96% |
| Inability to Feel | MADRS | 36 | 12 | 24 | 66.67% |
| Inner Tension | MADRS | 26 | 11 | 15 | 57.69% |
| Pessimistic Thoughts | MADRS | 28 | 7 | 21 | 75.00% |
| Suicidal Thoughts | MADRS | 11 | 3 | 8 | 72.72% |
| Guilt Feelings | BPRS | 34 | 14 | 20 | 58.82% |
| Tension | BPRS | 16 | 11 | 5 | 31.25% |
| Anxiety | BPRS | 37 | 19 | 18 | 48.65% |
| Emotional Withdrawal | BPRS | 13 | 8 | 5 | 38.46% |
| Blunted Affect | BPRS | 15 | 11 | 4 | 26.67% |

TABLE 2
B Scores recorded against relevant MADRS and BPRS items for patients
as-signed to intra-day individualised dosing regimen (IDR). The
item scores represent the sum of the individual patient scores
for all patients (n = 8) in the IDR group. Assessment on day 1.

| | | | | | Percent- |
|---|---|---|---|---|---|
| | | Base- | | Improve- | age |
| | | line | | ment at | improve- |
| Scale item | Scale | IDR | Day 1 | Day 1 | ment |
| Reduced Sleep | MADRS | 25 | 12 | 13 | 52.00% |
| Concentration | MADRS | 30 | 1 | 29 | 96.67% |
| Difficulties | | | | | |
| Lassitude | MADRS | 27 | 5 | 22 | 81.48% |
| Inability to Feel | MADRS | 36 | 2 | 34 | 94.44% |
| Inner Tension | MADRS | 26 | 6 | 20 | 76.92% |
| Pessimistic | MADRS | 28 | 4 | 24 | 85.71% |
| Thoughts | | | | | |
| Suicidal Thoughts | MADRS | 11 | 1 | 10 | 90.91% |
| Guilt Feelings | BPRS | 34 | 11 | 23 | 67.65% |
| Tension | BPRS | 16 | 11 | 5 | 31.25% |
| Anxiety | BPRS | 37 | 16 | 21 | 56.76% |
| Emotional | BPRS | 13 | 8 | 5 | 38.46% |
| Withdrawal | | | | | |
| Blunted Affect | BPRS | 15 | 8 | 7 | 46.67% |

TABLE 2
C Scores recorded against relevant MADRS and BPRS items for patients
as-signed to intra-day individualised dosing regimen (IDR). The
item scores represent the sum of the individual patient scores
for all patients (n = 8) in the IDR group. Assessment on day 7.

| | | | | | Percent |
|---|---|---|---|---|---|
| | | Base- | | Improve- | age |
| | | line | Day | ment at | improve- |
| Scale item | Scale | IDR | 7 | Day 7 | ment |
| Reduced Sleep | MADRS | 25 | 9 | 16 | 64.00% |
| Concentration | MADRS | 30 | 9 | 21 | 70.00% |
| Difficulties | | | | | |
| Lassitude | MADRS | 27 | 3 | 24 | 88.89% |
| Inability to Feel | MADRS | 36 | 6 | 30 | 83.33% |
| Inner Tension | MADRS | 26 | 12 | 14 | 53.85% |
| Pessimistic Thoughts | MADRS | 28 | 3 | 25 | 89.29% |
| Suicidal Thoughts | MADRS | 11 | 3 | 8 | 72.73% |
| Guilt Feelings | BPRS | 34 | 10 | 24 | 70.59% |
| Tension | BPRS | 16 | 10 | 6 | 37.50% |
| Anxiety | BPRS | 37 | 17 | 20 | 54.05% |
| Emotional Withdrawal | BPRS | 13 | 8 | 5 | 38.46% |
| Blunted Affect | BPRS | 15 | 8 | 7 | 46.67% |

TABLE 3
A Scores recorded against relevant MADRS and BPRS items for
patients as-signed to 12 mg dosing regimen. The item scores
represent the sum of the individual patient scores for all
patients (n = 4) in the 12 mg group. Assessment 2 hours
(MADRS) or 3 hours (BPRS) after administration of the dose.

| | | | | Improve- | Percent- |
|---|---|---|---|---|---|
| | | Base- | | ment | age |
| | | line | 2/3 | after | improve- |
| Scale item | Scale | 12 mg | hours | 2/3 hours | ment |
| Concentration | MADRS | 16 | 7 | 9 | 56.25% |
| Difficulties | | | | | |
| Lassitude | MADRS | 16 | 10 | 6 | 37.50% |
| Inability to Feel | MADRS | 16 | 9 | 7 | 43.75% |
| Inner Tension | MADRS | 13 | 2 | 11 | 84.62% |
| Pessimistic | MADRS | 16 | 8 | 8 | 50.00% |
| Thoughts | | | | | |
| Suicidal Thoughts | MADRS | 8 | 3 | 5 | 62.50% |
| Guilt Feelings | BPRS | 18 | 9 | 9 | 50.00% |
| Tension | BPRS | 14 | 9 | 5 | 35.71% |
| Anxiety | BPRS | 25 | 11 | 14 | 56.00% |
| Emotional | BPRS | 13 | 11 | 2 | 15.38% |
| Withdrawal | | | | | |
| Blunted Affect | BPRS | 11 | 8 | 3 | 27.27% |

TABLE 3
B Scores recorded against relevant MADRS and BPRS items for
patients as-signed to 12 mg dosing regimen. The item scores
represent the sum of the individual patient scores for all
patients (n = 4) in the 12 mg group. Assessment on day 1.

| | | | | | Percent- |
|---|---|---|---|---|---|
| | | Base- | | Improve- | age |
| | | line | Day | ment at | improve- |
| Scale item | Scale | 12 mg | 1 | Day 1 | ment |
| Reduced Sleep | MADRS | 12 | 10 | 2 | 16.67% |
| Concentration | MADRS | 16 | 2 | 14 | 87.50% |
| Difficulties | | | | | |
| Lassitude | MADRS | 16 | 0 | 16 | 100.00% |
| Inability to Feel | MADRS | 16 | 1 | 15 | 93.75% |
| Inner Tension | MADRS | 13 | 3 | 10 | 76.92% |
| Pessimistic | MADRS | 16 | 7 | 9 | 56.25% |
| Thoughts | | | | | |
| Suicidal Thoughts | MADRS | 8 | 5 | 3 | 37.50% |
| Guilt Feelings | BPRS | 18 | 5 | 13 | 72.22% |
| Tension | BPRS | 14 | 6 | 8 | 57.14% |
| Anxiety | BPRS | 25 | 6 | 19 | 76.00% |
| Emotional | BPRS | 13 | 8 | 5 | 38.46% |
| Withdrawal | | | | | |
| Blunted Affect | BPRS | 11 | 6 | 5 | 45.45% |

TABLE 3
C Scores recorded against relevant MADRS and BPRS items for
patients as-signed to 12 mg dosing regimen. The item scores
represent the sum of the individual patient scores for all
patients (n = 4) in the 12 mg group. Assessment on day 7.

| | | | | | Percent- |
|---|---|---|---|---|---|
| | | Base- | | Improve- | age |
| | | line | Day | ment at | improve- |
| Scale item | Scale | 12 mg | 7 | Day 7 | ment |
| Reduced Sleep | MADRS | 12 | 6 | 6 | 50.00% |
| Concentration | MADRS | 16 | 3 | 13 | 81.25% |
| Difficulties | | | | | |
| Lassitude | MADRS | 16 | 3 | 13 | 81.25% |
| Inability to Feel | MADRS | 16 | 1 | 15 | 93.75% |
| Inner Tension | MADRS | 13 | 5 | 8 | 61.54% |
| Pessimistic | MADRS | 16 | 8 | 8 | 50.00% |
| Thoughts | | | | | |
| Suicidal Thoughts | MADRS | 8 | 7 | 1 | 12.50% |
| Guilt Feelings | BPRS | 18 | 5 | 13 | 72.22% |
| Tension | BPRS | 14 | 6 | 8 | 57.14% |
| Anxiety | BPRS | 25 | 6 | 19 | 76.00% |
| Emotional | BPRS | 13 | 6 | 7 | 53.85% |
| Withdrawal | | | | | |
| Blunted Affect | BPRS | 11 | 5 | 6 | 54.55% |

### Example 8—Clinical Trial of 5-MeO-DMT Administered Via Inhalation to Patients with Postpartum Depression

The single-arm, open-label clinical trial will involve 15 adult female patients with clinically diagnosed postpartum depression (PPD).

The patients will receive a single-day individualized 5-MeO-DMT dosing regimen via inhalation after vaporization.

More in particular, the patients will receive up to three doses of 5-MeO-DMT on Day 0: 6 mg, 12 mg, and 18 mg.
1. All patients will receive an initial dose of 6 mg 5-MeO-DMT.
2. The second dose (12 mg) will only be administered if:
   a. A peak experience (total score of ⋅ 75) has not been achieved following the 6 mg dose, and
   b. The 6 mg dose was safe and well-tolerated according to the investigator,
   c. Any psychoactive effects (PsE) of the prior dose have subsided, and
   d. Pre-dose vital parameters and forced expiratory volume in one second (FEV1) are in normal range, or if outside of the normal range, are not clinically significant according to the investigator.
3. Similarly, a third dose (18 mg) will only be administered if:
   a. A peak experience (total score of ⋅ 75) has not been achieved following the 12 mg dose, and
   b. The 12 mg dose was safe and well-tolerated according to the investigator, and
   c. Any PsE of the prior dose have subsided, and
   d. Pre-dose vital parameters and forced expiratory volume in one second (FEV1) are in normal range, or if outside of the normal range, are not clinically significant according to the investigator.

The patients will be assessed for a peak psychedelic experience (based on a patient-scored visual analogue scale, the PE scale), sedation, and other endpoints after dosing. Follow-up visits are planned for Day 1, and Day 7 after the dosing day.

The following criteria must be met by all patients considered for clinical trial participation:
1. Is female and in the age range between 18 and 45 years (inclusive) at screening.
2. Has a body mass index (BMI) in the range of 18.5 and 35 kg/m² (inclusive) at screening.
3. Meets the trial criteria for PPD as assessed by a trial psychiatrist or registered psychologist:
   a. Diagnosis of Major Depressive Disorder without psychotic features, confirmed by the Mini-International Neuropsychiatric Interview (MINI), with peri-partum onset that began no earlier than gestation and no later than the first 4 weeks postpartum.
   b. Has a Montgomery-Åsberg Depression Rating Scale (MADRS) total score of equal to or greater than 28 at screening and pre-dose on Day 0.
6. Must have either ceased lactating at screening; or, if still lactating or actively breast feeding at screening, must agree to temporarily cease breastfeeding from just prior to receiving study drug on Day 0 through 24 hours post last dose, and to pump and discard all breastmilk during those 24 hours as needed, but need to include a pump/discard at 2.5 hours post last dose and 24 hours post last dose prior to reinitiating breastfeeding.
4. Must agree to remain completely abstinent (complete avoidance of heterosexual intercourse) or use a highly effective (failure rate <1%), medically accepted contraceptive method for 30 days prior to dosing and for 90 days after 5-MeO-DMT dosing. Patients must have a negative pregnancy test at screening and on the pre-test day (Day −1).
5. Is willing to delay start of other antidepressant or anxiety medication until after the end of the trial at Day 7 and agrees to keep any psychotherapy unchanged during the trial.

A potential patient who meets any of the following key exclusion criteria will be excluded from participation in this trial:
1. Has, based on history, psychiatric assessment, and evaluation of the MINI, a current or prior diagnosis of bipolar disorder, a manic or hypomanic episode, a psychotic disorder, Major Depressive Disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed bipolar disorder, psychotic disorder or other mood disorder (including MDD) with psychotic features.
3. Is in the judgement of a trial psychiatrist or registered psychologist, at significant risk for suicide based on history, psychiatric assessment, and evaluation of suicidal ideation and suicidal behaviour based on the Columbia-Suicide Severity Rating Scale (C-SSRS).
4. Has taken anti-depressive medication within 14 days or 5 half-lives (whichever is longer) prior to dosing (exception: within the last 5 weeks in the case of fluoxetine).
5. Has taken any other medication with monoamine oxidase inhibitor (MAOI) activity within 14 days or 5 half-lives (whichever is longer) prior to dosing.
6. Has previously experienced a significant adverse reaction to a hallucinogenic or psychedelic drug (e.g., psilocybin, Psilocybe spp. mushrooms, 5-MeO-DMT, DMT, ayahuasca, LSD, mescaline) according to the investigator's judgment.
7. Has known allergies or hypersensitivity or any other contraindication to 5-MeO-DMT.
8. Has any current or past clinically significant condition (e.g., severe infection, pulmonary disease, ⋅uncontrolled⋅hypertension, new onset of hypertensive disorders of pregnancy during pregnancy or in the postnatal period (e.g., gestational hypertension, pre-eclampsia-eclampsia, superimposed pre-eclampsia), uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that renders the patient unsuitable for the trial according to the investigator's-judgment.
9. Takes any medication or other substance that renders the patient unsuitable for the trial according to the investigator's judgment.
10. Has a clinically significant abnormality in physical examination, vital signs, ECG, or clinical laboratory parameters which renders the patient unsuitable for the trial according to the investigator's judgment.
11. Patient who has a positive pregnancy test at screening or on the pre-test day (Day −1), is pregnant, or plans to become pregnant during the course of the trial and up to 90 days after 5-MeO-DMT dosing.
12. Patients with DSM-5 drug or alcohol use disorder within 6 months prior to screening.

The primary objective of the trial is to determine the onset and 7-day durability of anti-depressive effects of a single-day individualized dosing regimen of 6 mg, 12 mg and 18 mg of 5-MeO-DMT in adult, female patients with PPD.

Secondary objectives are to determine the anti-depressive effects; the anti-anxiety effects; the effects on maternal behavior; the safety and tolerability; the intensity and duration of psychoactive effects (PsE); the impact on cognitive outcome of a single-day individualized dosing regimen of 6 mg, 12 mg and 18 mg of 5-MeO-DMT in adult, female patients with PPD.

An exploratory objective is to determine in breastmilk, blood and urine the amount of 5-MeO-DMT and metabolites, bufotenine and 5-methoxyindole-3-acetic acid (5-MIAA), measured by LC/MS/MS (metabolite identification screening may be performed, as required), following dose administration of a single-day DR of 6 mg, 12 mg and 18 mg of 5-MeO-DMT in adult, female patients with PPD.

The primary endpoint of the study is the evaluation of the anti-depressive effects of 5-MeO-DMT by the change from baseline in MADRS assessed at Day 7.

Secondary endpoints include the anti-depressive effects of 5-MeO-DMT evaluated by
The anti-depressive effects of 5-MeO-DMT evaluated by:
   The proportion of patients in remission (MADRS·10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (⋅ 50% reduction from baseline in MADRS total score) at 2 hours after the final study drug dosing on Day 0, and at Day 1 and Day 7;
   Change from baseline in Clinical Global Impression-Severity scale (CGI-S) 2 hours after final study drug dosing on Day 0, and at Day 1 and Day 7;
Effects on maternal behaviour as assessed by the change from baseline in the Barkin Index of Maternal Functioning (BIMF) total and sub-scale scores to Day 7;
Exposure of 5-MeO-DMT and bufotenine in breastmilk obtained at the pre-test day (Day −1), 1 hour after last study drug dosing, at discharge, in the evening of Day 0, and on Day 1 and on Day 7;
Exposure of 5-MeO-DMT and bufotenine in blood obtained at the pre-test day (Day −1), 1 hour after last study drug dosing, at discharge, on Day 1 and on Day 7;
The safety and tolerability of 5-MeO-DMT evaluated by:
   Reporting of treatment-emergent adverse events (TEAEs);
   Clinically significant changes from baseline in ECG, vital signs, safety laboratory assessments, peak flow respirometry;
   Assessment of sedation (Modified Observer's Assessment of Alertness and Sedation scale [MOAA/S]) following each dose (when the PsE has subsided and 60 minutes after each study drug dosing) and as part of the discharge evaluation on Day 0;
   Change from baseline in Clinician Administered Dissociative States Scale (CADSS) assessed as part of the discharge evaluation on Day 0 and at Day 1 and Day 7;
   Change from baseline in Brief Psychiatric Rating Scale (BPRS) assessed as part of the discharge evaluation on Day 0, and at Day 1 and Day 7;
   Change from baseline in C-SSRS assessed as part of the discharge evaluation on Day 0, and at Day 1 and Day 7;
   Change from baseline in YMRS assessed as part of the discharge evaluation on Day 0, and at Day 1 and Day 7;
The PsE experienced by the patients as reported 30 to 60 minutes after each dosing, when the PsE has subsided:
   PsE assessment using the peak experience (PE) scale to assess the achievement of a PE (PE scale total score ⋅ 75);
   Challenging Experience Questionnaire (CEQ);
   Mystical Experience Questionnaire (MEQ-30);
Duration of the PsE defined as the time from study drug dosing to the time when the PsE have subsided (investigator- and patient-scored), completed 30 to 60 minutes after each dosing;

One patient with postpartum depression diagnosed by a psychiatrist has, so far, been recruited into the clinical trial. Diagnosis was Major Depressive Disorder without psychotic features, confirmed by the Mini-International Neuropsychiatric Interview (MINI) (v7.0.2), with peri-partum onset that began no earlier than gestation and no later than the first 4 weeks postpartum. The patient was diagnosed with postpartum depression after giving birth to her third child. The patient completed all planned visit days. The inhalation procedure was adequately performed by the patient and was well tolerated with no inhalation-related adverse events.

### Results

Except for a temporary, clinically non-relevant increase in heart rate and blood pressure shortly after administration of 5-MeO-DMT, no other noteworthy changes in vital parameters occurred. Assessments of ECG (at 3 hours after administration) and safety laboratory analyses (at 7 days), CADSS (at 3 hours, 1 day and 7 days) were unremarkable. The few reported adverse events (cramping left abdominal pain and headache, both on Day 0) were mild, short-lasting and resolved spontaneously by the end of the study.

With regard to the intensity of the psychedelic experience, the recorded PES score achieved upon exposure to a nominal dose of 6 mg was 17.3. This score indicated the need to proceed to the administration of a subsequent, higher dose of 12 mg, per the design of the individualised dosing regimen. The PES score achieved for this dose was 85.7 and, being ⋅ 75, indicated the occurrence of a peak psychedelic experience and the completion of the IDR for this patient.

Significantly, the patient reported a major improvement in her depressive symptoms as assessed by MADRS at the earliest assessment timepoint of 2 hours after drug administration, with the effect being maintained over time (Table 4). The patient also fulfilled standard criteria for MADRS response (at least 50% improvement from baseline) and MADRS remission (MADRS total score equal or less than 10).

TABLE 4
MADRS/BPRS scores table

| | | 2 hr | | | | | |
|---|---|---|---|---|---|---|---|
| | | (MADRS); | | | Improvement | | |
| | | Discharge | | | 2 hr; | | |
| Scale item | Baseline | (BPRS) | Day 1 | Day 7 | Discharge | Day 1 | Day 7 |
| MADRS | | | | | | | |
| Reported sadness | 4 | 0 | 0 | 0 | 4 | 4 | 4 |
| Apparent sadness | 5 | 0 | 0 | 0 | 5 | 5 | 5 |
| Inner Tension | 5 | 0 | 0 | 1 | 5 | 5 | 4 |
| Reduced Sleep | 5 | 5 | 0 | 0 | 0 | 5 | 5 |
| Reduced appetite | 0 | 0 | 0 | 0 | 0 | 0 | 0 |
| Concentration Difficulties | 0 | 0 | 0 | 0 | 0 | 0 | 0 |
| Lassitude | 3 | 0 | 0 | 0 | 3 | 3 | 3 |
| Inability to Feel | 3 | 0 | 0 | 0 | 3 | 3 | 3 |
| Pessimistic Thoughts | 4 | 0 | 0 | 0 | 4 | 4 | 4 |
| Suicidal Thoughts | 0 | 0 | 0 | 0 | 0 | 0 | 0 |
| Total MADRS | 29 | 5 | 0 | 1 | 24 | 29 | 28 |
| BPRS | | | | | | | |
| Somatic Concern | 6 | 1 | 1 | 1 | 5 | 5 | 5 |
| Anxiety | 7 | 1 | 1 | 1 | 6 | 6 | 6 |
| Emotional withdrawal | 5 | 1 | 1 | 1 | 4 | 4 | 4 |
| Conceptual | 1 | 1 | 1 | 1 | 0 | 0 | 0 |
| disorganization | | | | | | | |
| Guilt feelings | 6 | 1 | 1 | 1 | 5 | 5 | 5 |
| Tension | 6 | 1 | 1 | 1 | 5 | 5 | 5 |
| Mannerisms and | 1 | 1 | 1 | 1 | 0 | 0 | 0 |
| Posturing | | | | | | | |
| Grandiosity | 1 | 1 | 1 | 1 | 0 | 0 | 0 |
| Depressive Mood | 6 | 1 | 1 | 1 | 5 | 5 | 5 |
| Hostility | 1 | 1 | 1 | 1 | 0 | 0 | 0 |
| Suspiciousness | 1 | 1 | 1 | 1 | 0 | 0 | 0 |
| Hallucinatory behaviour | 1 | 1 | 1 | 1 | 0 | 0 | 0 |
| Motor retardation | 1 | 1 | 1 | 1 | 0 | 0 | 0 |
| Uncooperativeness | 1 | 1 | 1 | 1 | 0 | 0 | 0 |
| Unusual Thought Content | 1 | 1 | 1 | 1 | 0 | 0 | 0 |
| Blunted affect | 1 | 1 | 1 | 1 | 0 | 0 | 0 |
| Excitement | 2 | 1 | 1 | 1 | 1 | 1 | 1 |
| Disorientation | 1 | 1 | 1 | 1 | 0 | 0 | 0 |

Significant improvements were noted for several MADRS items in particular. The items are outlined in Table 4. While the patient's baseline scores for some items reflected absence of the symptom (reduced appetite, concentration difficulties, suicidal thoughts), items with scores reflecting severe symptoms (e.g., reduced sleep, inner tension) saw remarkable improvement.

Similarly, improvements were seen in several BPRS items, including Somatic Concerns, Anxiety, Emotional withdrawal, Guilt feelings and Tension.

Additionally, improvements in maternal functioning were evidenced by improvements in the BIMF score recorded at Day 7, as outlined in Table 5, with the total score improving by 14% from 92 to 105 (out of a possible total of 120).

Several functional domains of maternal function were also assessed, as defined by Barkin et al. The improvements in each functional domain are outlined in more detail in Table 6.

Here, noteworthy improvements in self-care, psychological well-being and management were achieved, with percentage improvements ranging from 18% (management) to (44%),% (self-care). These improvements reinforce the relationship between improvement in depressive items, as assessed by the MADRS, and improvements in maternal functioning.

It is noted that the patient scored comparatively high already before treatment. In some functional domains, the score was at the maximum value, or close to it (see Table 6), so that the scope for improvement by therapy was limited.

TABLE 5
BIMF scores table

| | | Day | Day | Δ |
|---|---|---|---|---|
| No. | BIM F item | 0 | 7 | (Day 7) |
| 1 | I am a good mother | 6 | 6 | 0 |
| 2 | I feel rested | 0 | 2 | 2 |
| 3 | I am comfortable with the way I've | 6 | 6 | 0 |
| | chosen to feed my baby (either bottle | | | |
| | or breast, or both) | | | |
| 4 | My baby and I understand each other | 5 | 5 | 0 |
| 5 | I am able to relax and enjoy time | 5 | 6 | 1 |
| | with my baby | | | |
| 6 | There are people in my life that I | 6 | 6 | 0 |
| | can trust to care for my baby when | | | |
| | I need a break | | | |
| 7 | I am comfortable allowing a trusted | 6 | 6 | 0 |
| | friend or relative to care for my | | | |
| | baby (can include baby's father | | | |
| | or partner) | | | |
| 8 | I am getting enough adult interaction | 6 | 6 | 0 |
| 9 | I am getting enough encouragement | 5 | 6 | 1 |
| | from other people | | | |
| 10 | I trust my own feelings (instincts) | 5 | 6 | 1 |
| | when it comes to taking care of my | | | |
| | baby | | | |
| 11 | I take a little time each week to do | 5 | 6 | 1 |
| | something for myself | | | |
| 12 | I am taking good care of my baby's | 6 | 6 | 0 |
| | physical needs (feedings, changing | | | |
| | diapers, doctor's appointments) | | | |
| 13 | I am taking good care of my physical | 4 | 5 | 1 |
| | needs (eating, showering, etc) | | | |
| 14 | I make good decisions about my baby's | 6 | 6 | 0 |
| | health and well being | | | |
| 15 | My baby and I are getting into a | 5 | 6 | 1 |
| | routine | | | |
| 16 | I worry about how other people judge | 3 | 5 | 2 |
| | me (as a mother) | | | |
| 17 | I am able to take care of my baby and | 5 | 5 | 0 |
| | my other responsibilities | | | |
| 18 | Anxiety or worry often interferes with | 2 | 5 | 3 |
| | my mothering ability | | | |
| 19 | As time goes on, I am getting better | 0 | 1 | 1 |
| | at taking care of my baby | | | |
| 20 | I am satisfied with the job I am doing | 6 | 5 | −1 |
| | as a new mother | | | |
| | TOTAL | 92 | 105 | 13 |

TABLE 6
BIMF functional domain scores table

| | | Score | | | | |
|---|---|---|---|---|---|---|
| Functional | Related BIMF | Maximum | Day | Day | Δ | % |
| Domain | Items | Possible | 0 | 7 | (Day 7) | Improvement |
| Self Care | 2, 11, 13 | 18 | 9 | 13 | 4 | 44.44% |
| Infant Care | 12, 14 | 12 | 12 | 12 | 0 | 0.00% |
| Mother-Child | 4, 5, 15 | 18 | 15 | 17 | 2 | 13.33% |
| Interaction | | | | | | |
| Psychological | 1, 2, 3, 5, 7, 10, 11, | 60 | 44 | 53 | 9 | 20.45% |
| Well-being | 16, 18, 20 | | | | | |
| Social Support | 6, 8, 9 | 18 | 17 | 18 | 1 | 5.88% |
| Management | 7, 11, 13, 14, 17, 18 | 36 | 28 | 33 | 5 | 17.86% |
| Adjustment | 17, 19 | 12 | 5 | 6 | 1 | 20.00% |

### Summary and Conclusions

A. An individualised dosing regimen of 6 mg 5-MeO-DMT, followed by 12 mg 5-MeO-DMT administered via inhalation was well tolerated and induced an astonishing and very significant clinical response in a patient formally diagnosed with postpartum depression.

B. The clinical response occurs rapidly within 2 hours after 5-MeO-DMT administration. Such rapid onset is unusual and has not been seen with conventional classes of antidepressants, including tricyclic antidepressants, monoamine oxidase inhibitors, selective serotonin-reuptake inhibitors (SSRls), serotonin-norepinephrine reuptake inhibitors (SNRls), and others, which generally take 4 to 6 weeks to show their effect.

C. The patient experienced a clinical remission within 2 hours after 5-MeO-DMT administration according to the IDR. This is highly superior to any approved therapy for postpartum depression, and also to all previously tested psychedelic agents.

D. A significant clinical response was sustained over the 7-day follow-up period, although 5-MeO-DMT was only given once and is no longer efficaciously present in the body during this time frame (see pharmacokinetic data in Example 6 above).

This observation supports the superior clinical profile of 5-MeO-DMT and allows for convenient administration intervals.

E. In addition to anti-depressive effects, endpoints assessing other symptoms (such as somatic concerns, emotional withdrawal, anxiety, guilt and tension) were positively impacted, supporting the use of 5-MeO-DMT in patients with other mental diseases.

F. In addition to anti-depressive effects, endpoints assessing maternal functioning, as assessed using the BIMF, such as self-care, psychological well-being and management, were positively impacted. This supports additional benefits of 5-MeO-DMT to patients suffering from PPD beyond improvement in their core depressive symptoms.

The highlighted aspects show that 5-MeO-DMT has a significantly improved efficacy profile compared to approved pharmacological therapies for postpartum depression and to all previously tested psychedelic agents, when used according to the present invention.

Together with the short duration of the acute psychedelic effects and the favourable safety profile, these data show that the technical problem to provide an improved psychoactive therapy in a patient with a postpartum depression is solved by the present invention.

### Example 9—Clinical Trial of 5-MeO-DMT Administered Via Inhalation to Patients with Bipolar II Disorder

The single-arm, open-label clinical trial will involve 15 adult patients with bipolar II disorder and a current major depressive episode.

Patients who are currently taking anti-depressive medication need to discontinue or taper over time such medication.

The patients will receive a single-day individualized 5-MeO-DMT dosing regimen via inhalation after vaporization.

More in particular, the patients will receive up to three doses of 5-MeO-DMT on the administration day (Day 0): 6 mg, 12 mg, and 18 mg.
1. All patients will receive an initial dose of 6 mg 5-MeO-DMT.
2. The second dose (12 mg) will only be administered if:
   a. A peak experience (PES total score of ⋅ 75) has not been achieved following the 6 mg dose, and
   b. The 6 mg dose was safe and well-tolerated.
3. Similarly, a third dose (18 mg) will only be administered if:
   a. A peak experience (PES total score of ⋅ 75) has not been achieved following the 12 mg dose, and
   b. The 12 mg dose was safe and well-tolerated.

The patients will be assessed for a peak psychedelic experience based on the patient-scored PES described above, sedation and other endpoints after dosing. Follow-up visits are planned for Day 1, and Day 7 after the dosing day.

Selection of patients is based on the following key inclusion criteria:
1. Understands the nature of the clinical trial and has provided signed and dated written informed consent in accordance with local regulations before the conduct of any trial-related procedures.
2. Is male or female and in the age range between 18 and 64 years (inclusive) at screening.
3. Meets the trial criteria for bipolar II disorder and is experiencing a major depressive episode, as assessed by a trial psychiatrist or registered clinical psychologist:
   a. Meets the Diagnostic and Statistical Manual of Mental Disorders (DSM-5) diagnostic criteria for bipolar II disorder with a current major depressive disorder episode confirmed by the Mini-International Neuropsychiatric Interview (MINI);
   b. Has a Montgomery-Åsberg Depression Rating Scale (MADRS) total score of equal to or greater than 24 at screening and prior to first dose on Day 0;
4. Has a Young Mania Rating Scale (YMRS) total score less than or equal to 8 at screening and prior to first dose on Day 0;
5. Agrees to keep any psychotherapy unchanged, and not initiate any new psychoactive medications during the course of the trial.
6. Female patients must be either surgically sterile (hysterectomy, tubal ligation, or bilateral oophorectomy (6 months prior to screening)) or postmenopausal with amenorrhea for the last 2 years or remain completely abstinent (complete avoidance of heterosexual intercourse) or use a highly effective (failure rate <1%) medically accepted contraceptive method, including, but not limited to, bilateral tubal ligation/occlusion, hormone contraceptives that inhibit ovulation, intrauterine device (including hormone-releasing intrauterine device/systems) for 30 days before and 90 days after 5-MeO-DMT dosing and must have a negative serum pregnancy test at screening and a negative urine pregnancy test on the pre-test day (Day −1).
7. Male patients must use prophylactic contraception (i.e., condom with spermicide or abstinence) and must not donate sperm for 30 days after 5-MeO-DMT dosing.

A potential patient who meets any of the following key exclusion criteria will be excluded from participation in this trial:
1. Has, based on history, psychiatric assessment, and evaluation of the MINI, a current or prior diagnosis of bipolar I disorder, a manic episode, a psychotic disorder, major depressive disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed psychotic disorder, bipolar I disorder, or MDD with psychotic features.
3. Has significant suicide risk as defined by: (a) suicidal ideation as endorsed on items 4 or 5 on the C-SSRS within the past year, during the screening period, or at Baseline; or (b) suicidal behaviours within the past year; or (c)-clinical assessment of significant suicidal risk during clinical interview; or (d) non-suicidal self-injury within the past year.
4. Has taken anti-depressive medication within 7 days or 5 half-lives (whichever is longer) prior to dosing (exception: within the last 5 weeks in the case of fluoxetine).
5. Has taken a medication with monoamine oxidase inhibitor (MAOI) activity within 14 days or 5 half-lives (whichever is longer) prior to dosing.
6. Has taken mood stabilizer therapy (e.g., lamotrigine, valproate, atypical antipsychotics) within 14 days (28 days for lithium) or 5 half-lives (whichever is longer) prior to dosing or takes mood stabilizer therapy at screening or is expected to require mood stabilizer therapy during the study (as per the investigator's judgment).
7. Has previously experienced a significant adverse reaction to a hallucinogenic or psychedelic drug according to the investigator's judgment.
8. Has known allergies or hypersensitivity or any other contraindication to 5-MeO-DMT.
9. Has any current or past clinically significant condition (e.g., severe infection, severe pulmonary disease, uncontrolled-hypertension, uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that may interfere with the interpretation of the trial results, constitutes a health risk for the patient, or that otherwise renders the patient unsuitable for the trial according to the investigator's⋅judgment.
10. Takes any medication or other substance that renders the patient unsuitable for the trial according to the investigator's judgment.
11. Has a clinically significant abnormality in physical examination, vital signs, electrocardiogram (ECG), or clinical laboratory parameters which renders the patient unsuitable for the trial according to the investigator's judgment.
12. Female patient who has a positive pregnancy test at screening or on the pre-test day (Day −1), is pregnant or lactating, or plans to become pregnant during the course of the trial and up to 30 days after 5-MeO-DMT dosing.
13. Patients with DSM-5, alcohol or substance use disorder (excluding tobacco or caffeine use disorders) within 6 months prior to screening.

The primary objective of the trial is to determine the onset and durability of anti-depressive effects of a single-day individualized dosing regimen of 6 mg, 12 mg and 18 mg of 5-MeO-DMT in patients with bipolar II disorder and a current major episode of depression. Secondary objectives are to determine the effect on depressive symptoms and global clinical status; the safety and tolerability; the intensity and duration of psychoactive effects (PsE); the impact on sleep quality; the impact on cognitive outcomes of a single-day individualized dosing regimen of 6 mg, 12 mg and 18 mg of 5-MeO-DMT in patients with bipolar II disorder and a current major episode of depression.

The primary endpoint of the study is the evaluation of the anti-depressive effects of 5-MeO-DMT by the change from baseline in MADRS assessed at Day 7.

Secondary endpoints include:
The anti-depressive effects of 5-MeO-DMT administered via inhalation evaluated by:
   The proportion of patients in remission (MADRS ⋅ 10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (⋅ 50% reduction from baseline in MADRS total score) at 2 hours after the final study drug dosing on Day 0, and at Day 1 and Day 7;
   Change from baseline in CGI-S at 2 hours after the final study drug dosing on Day 0, and at Day 1 and Day 7.
   from baseline in BDRS at Day 1 and Day 7
The safety and tolerability of 5-MeO-DMT administered via inhalation evaluated by:
   Reporting of treatment-emergent adverse events (TEAEs);
   Clinically significant changes from baseline in ECG, vital signs, safety laboratory assessments and spirometry assessments;
   Assessment of sedation (Modified Observer's Assessment of Alertness and Sedation scale [MOAA/S]) following each dose (when the PsE have subsided and 60 minutes after each study drug dosing) and as part of the discharge evaluation on Day 0;
   The incidence of adverse events (AEs) of mania or hypomania (as assessed using the DSM-5 criteria for mania/hypomania);
   Change from baseline in YMRS assessed as part of the discharge evaluation on Day 0 and at Day 1 and Day 7;
   Change from baseline in Clinician Administered Dissociative States Scale (CADSS) assessed as part of the discharge evaluation on Day 0 and at Day 1 and Day 7;
   Assessment of patient discharge readiness at discharge on Day 0 using the Clinical Assessment of Discharge Readiness (CADR);
   Change from baseline in Brief Psychiatric Rating Scale (BPRS) assessed as part of the discharge evaluation on Day 0, and at Day 1 and Day 7;
   C-SSRS categorization based on the Columbia Classification Algorithm of Suicide Assessment (C-CASA).
The PsE experienced by the patients as reported 30 to 60 minutes after each dosing, when the PsE has subsided:
   PsE assessment using the peak experience (PE) Scale (PES) to assess the achievement of a PE (PES total score ⋅ 75);
   Challenging Experience Questionnaire (CEQ);
   Mystical Experience Questionnaire (MEQ-30).
Duration of the PsE, defined as the time from study drug dosing to the time when the PsE have subsided, completed 30 to 60 minutes after each dosing.
The impact on sleep quality as evaluated by change from pre-test day (Day −1) to Day 1 and to Day 7 in the Pittsburgh Sleep Quality Index (PSQI).
The impact on cognitive outcomes as evaluated by change from the pre-test day (Day −1) to discharge on Day 0, to Day 1 and to Day 7 in:
   Rapid visual information processing (RVP) test;
   Verbal recognition memory (VRM) test;
   Spatial working memory (SWM) test;
   Digit symbol substitution test (DSST).

1. 5-Methoxy-N,N-dimethyltryptamine (5-MeO-DMT) or a pharmaceutically acceptable salt thereof for use in treating a patient suffering from negative thinking.

2. 5-MeO-DMT or a pharmaceutically acceptable salt thereof for use as in claim 1, wherein the treatment reduces or eliminates negative thinking.

3. 5-MeO-DMT or a pharmaceutically acceptable salt thereof for use as in claim 2, wherein the treatment reduces or eliminates feelings of worthlessness, helplessness and hopelessness, and/or guilt.

4. 5-MeO-DMT or a pharmaceutically acceptable salt thereof for use as in claim 2 or 3, wherein the reduction or elimination of negative thinking is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

5. 5-MeO-DMT or a pharmaceutically acceptable salt thereof for use as in claim 2 or 3, wherein the reduction or elimination of negative thinking occurs not later than about 2 hours after the last administration of 5-MeO-DMT or a pharmaceutically acceptable salt thereof and wherein the reduction or elimination of negative thinking persists until at least 6 days after the last administration of 5-MeO-DMT or a pharmaceutically acceptable salt thereof; in particular until at least 14 days; more preferably until at least 28 days after the last administration of 5-MeO-DMT or a pharmaceutically acceptable salt thereof.

6. 5-MeO-DMT or a pharmaceutically acceptable salt thereof for use as in claims 1 to 5, wherein the patient is suffering from a mental or nervous system disorder associated with the negative thinking.

7. 5-MeO-DMT or a pharmaceutically acceptable salt thereof for use as in claim 6, wherein the patient suffering from a mental or nervous system disorder suffers from a treatment resistant form of the disorder.

8. 5-MeO-DMT or a pharmaceutically acceptable salt thereof for use as in claims 1 to 5, wherein the patient is suffering from a disorder characterized by depressive episodes associated with the negative thinking.

9. 5-MeO-DMT or a pharmaceutically acceptable salt thereof for use as in claim 8, wherein the patient suffering from a disorder characterized by depressive episodes suffers from a treatment resistant form of the disorder.

10. 5-MeO-DMT or a pharmaceutically acceptable salt thereof for use as in claims 1 to 5, wherein the patient is suffering from major depressive disorder (MDD) associated with the negative thinking.

11. 5-MeO-DMT or a pharmaceutically acceptable salt thereof for use as in claim 10, wherein the patient suffering from MDD suffers from a treatment resistant form of the disorder.

12. 5-MeO-DMT or a pharmaceutically acceptable salt thereof for use as in claims 1 to 5, wherein the patient is suffering from postpartum depression (PPD) associated with the negative thinking.

13. 5-MeO-DMT or a pharmaceutically acceptable salt thereof for use as in claim 12, wherein the patient suffering from PPD suffers from a treatment resistant form of the disorder.

14. 5-MeO-DMT or a pharmaceutically acceptable salt thereof for use as in claim 12 or 13, wherein the patient suffers in addition from compromised maternal functioning.

15. 5-MeO-DMT or a pharmaceutically acceptable salt thereof for use as in claim 14, wherein the patient has a Barkin Index of Maternal Functioning (BIMF) score of 80 or below, such as 65 or below.

16. 5-MeO-DMT or a pharmaceutically acceptable salt thereof for use as in claim 14 or 15, wherein the treatment improves maternal functioning.

17. 5-MeO-DMT or a pharmaceutically acceptable salt thereof for use as in claim 16, wherein the improvement in maternal functioning is reflected by an improvement of the BIMF total score by 10% or more, preferably by 20% or more.

18. 5-MeO-DMT or a pharmaceutically acceptable salt thereof for use as in claim 16 or 17, wherein the improvement in maternal functioning, is reflected by at least an improvement in the BIMF total score on day 7; on day 14; and/or on day 28 after the last administration of 5-MeO-DMT or a pharmaceutically acceptable salt thereof.

19. 5-MeO-DMT or a pharmaceutically acceptable salt thereof for use as in claim 16 or 17, wherein the improvement in maternal functioning, as reflected by at least an improvement in the BIMF total score, occurs not later than about 6 days after the last administration of 5-MeO-DMT or a pharmaceutically acceptable salt thereof and wherein the improvement in maternal functioning, as reflected by at least an improvement in the BIMF total score, persists until at least 14 days after the last administration of 5-MeO-DMT or a pharmaceutically acceptable salt thereof; more preferably until at least 28 days after the last administration of 5-MeO-DMT or a pharmaceutically acceptable salt thereof.

20. 5-MeO-DMT or a pharmaceutically acceptable salt thereof for use as in claims 1 to 5, wherein the patient is suffering from bipolar disorder associated with the negative thinking.

21. 5-MeO-DMT or a pharmaceutically acceptable salt thereof for use as in claim 20, wherein the patient is suffering from bipolar II disorder associated with the negative thinking.

22. 5-MeO-DMT or a pharmaceutically acceptable salt thereof for use as in claim 20, wherein the patient is suffering from bipolar I disorder associated with the negative thinking.

23. 5-MeO-DMT or a pharmaceutically acceptable salt thereof for use as in claims 20 to 22, wherein the patient suffers from a current major depressive episode.

24. 5-MeO-DMT or a pharmaceutically acceptable salt thereof for use as in claims 20 to 23, wherein the patient suffering from bipolar disorder suffers from a treatment resistant form of the disorder.

25. 5-MeO-DMT or a pharmaceutically acceptable salt thereof for use as in claims 1 to 5, wherein the patient is suffering from seasonal affective disorder associated with the negative thinking.

26. 5-MeO-DMT or a pharmaceutically acceptable salt thereof for use as in claim 25, wherein the patient suffering from seasonal affective disorder suffers from a treatment resistant form of the disorder.

27. 5-MeO-DMT or a pharmaceutically acceptable salt thereof for use as in claims 1 to 5, wherein the patient is suffering from persistent depressive disorder associated with the negative thinking.

28. 5-MeO-DMT or a pharmaceutically acceptable salt thereof for use as in claim 27, wherein the patient suffering from persistent depressive disorder suffers from a treatment resistant form of the disorder.

29. 5-MeO-DMT or a pharmaceutically acceptable salt thereof for use as in claims 1 to 5, wherein the patient is suffering from an anxiety disorder associated with the negative thinking.

30. 5-MeO-DMT or a pharmaceutically acceptable salt thereof for use as in claim 29, wherein the patient suffering from an anxiety disorder suffers from a treatment resistant form of the disorder.

31. 5-MeO-DMT or a pharmaceutically acceptable salt thereof for use as in claims 1 to 5, wherein the patient is suffering from generalised anxiety disorder (GAD) associated with the negative thinking.

32. 5-MeO-DMT or a pharmaceutically acceptable salt thereof for use as in claim 31, wherein the patient suffering from GAD suffers from a treatment resistant form of the disorder.

33. 5-MeO-DMT or a pharmaceutically acceptable salt thereof for use as in claims 1 to 5, wherein the patient is suffering from social anxiety disorder (SAD) associated with the negative thinking.

34. 5-MeO-DMT or a pharmaceutically acceptable salt thereof for use as in claim 33, wherein the patient suffering from SAD suffers from a treatment resistant form of the disorder.

35. 5-MeO-DMT or a pharmaceutically acceptable salt thereof for use as in claims 1 to 5, wherein the patient is suffering from an obsessive compulsive or related disorder associated with the negative thinking.

36. 5-MeO-DMT or a pharmaceutically acceptable salt thereof for use as in claim 35, wherein the obsessive compulsive or related disorder is obsessive compulsive disorder (OCD).

37. 5-MeO-DMT or a pharmaceutically acceptable salt thereof for use as in claim 35 or 36, wherein the patient suffers from a treatment resistant form of the disorder.

38. 5-MeO-DMT or a pharmaceutically acceptable salt thereof for use as in claims 1 to 5, wherein the patient is suffering from body dysmorphic disorder (BDD) associated with the negative thinking.

39. 5-MeO-DMT or a pharmaceutically acceptable salt thereof for use as in claim 38, wherein the patient suffering from BDD suffers from a treatment resistant form of the disorder.

40. 5-MeO-DMT or a pharmaceutically acceptable salt thereof for use as in claims 1 to 5, wherein the patient is suffering from post-traumatic stress disorder (PTSD) associated with the negative thinking.

41. 5-MeO-DMT or a pharmaceutically acceptable salt thereof for use as in claim 40, wherein the patient suffering from PTSD suffers from a treatment resistant form of the disorder.

42. 5-MeO-DMT or a pharmaceutically acceptable salt thereof for use as in claims 1 to 5, wherein the patient is suffering from a pain disorder associated with the negative thinking.

43. 5-MeO-DMT or a pharmaceutically acceptable salt thereof for use as in claim 42, wherein the patient is suffering from chronic pain associated with the negative thinking.

44. 5-MeO-DMT or a pharmaceutically acceptable salt thereof for use as in claim 42 or 43, wherein the patient suffers from a treatment resistant form of the disorder.

45. 5-MeO-DMT or a pharmaceutically acceptable salt thereof for use as in claims 1 to 5, wherein the patient is suffering from a mental and behavioural disorder due to psychoactive substance use associated with the negative thinking.

46. 5-MeO-DMT or a pharmaceutically acceptable salt thereof for use as in claim 45, wherein the patient is suffering from substance use disorder (SUD) associated with the negative thinking.

47. 5-MeO-DMT or a pharmaceutically acceptable salt thereof for use as in claim 45 or 46, wherein the patient suffers from a treatment resistant form of the disorder.

48. 5-MeO-DMT or a pharmaceutically acceptable salt thereof for use as in claims 1 to 5, wherein the patient is suffering from a psychotic disorder associated with the negative thinking.

49. 5-MeO-DMT or a pharmaceutically acceptable salt thereof for use as in claim 48, wherein the patient suffering from a psychotic disorder suffers from a treatment resistant form of the disorder.

50. 5-MeO-DMT or a pharmaceutically acceptable salt thereof for use as in claims 1 to 5, wherein the patient is suffering from schizophrenia associated with the negative thinking.

51. 5-MeO-DMT or a pharmaceutically acceptable salt thereof for use as in claim 50, wherein the patient suffering from schizophrenia suffers from a treatment resistant form of the disorder.

52. 5-MeO-DMT or a pharmaceutically acceptable salt thereof for use as in claims 1 to 5, wherein the patient is suffering from dementia associated with the negative thinking.

53. 5-MeO-DMT or a pharmaceutically acceptable salt thereof for use as in claims 1 to 5, wherein the patient is suffering from Alzheimer's dementia associated with the negative thinking.

54. 5-MeO-DMT or a pharmaceutically acceptable salt thereof for use as in claims 1 to 5, wherein the patient is suffering from an eating disorder associated with the negative thinking.

55. 5-MeO-DMT or a pharmaceutically acceptable salt thereof for use as in claim 54, wherein the patient suffering from an eating disorder suffers from a treatment resistant form of the disorder.

56. 5-MeO-DMT or a pharmaceutically acceptable salt thereof for use as in claims 1 to 5, wherein the patient is suffering from attention deficit hyperactivity disorder (ADHD) associated with the negative thinking.

57. 5-MeO-DMT or a pharmaceutically acceptable salt thereof for use as in claim 56, wherein the patient suffering from ADHD suffers from a treatment resistant form of the disorder.

58. 5-MeO-DMT or a pharmaceutically acceptable salt thereof for use as in claims 1 to 5, wherein the patient is suffering from a personality disorder associated with the negative thinking.

59. 5-MeO-DMT or a pharmaceutically acceptable salt thereof for use as in claim 58, wherein the patient is suffering from schizotypal personality disorder associated with the negative thinking.

60. 5-MeO-DMT or a pharmaceutically acceptable salt thereof for use as in claim 58 or 59, wherein the patient suffers from a treatment resistant form of the disorder.

61. 5-MeO-DMT or a pharmaceutically acceptable salt thereof for use as in claims 1 to 5, wherein the patient is suffering from a borderline personality disorder (BPD) associated with the negative thinking.

62. 5-MeO-DMT or a pharmaceutically acceptable salt thereof for use as in claim 61, wherein the patient suffering from an BPD suffers from a treatment resistant form of the disorder.

63. 5-MeO-DMT or a pharmaceutically acceptable salt thereof for use as in claims 1 to 5, wherein the negative thinking is due to traumatic brain injury.

64. 5-MeO-DMT or a pharmaceutically acceptable salt thereof for use as in claims 6 to 63, wherein the treatment leads to an improvement in the diagnosed disorder in a patient also suffering from associated negative thinking.

65. 5-MeO-DMT or a pharmaceutically acceptable salt thereof for use as in claim 64, wherein the improvement in the diagnosed disorder in a patient also suffering from associated negative thinking, as reflected by a reduction in the Clinical Global Impression-Severity (CGI-S) score, is observed about 2 hours after the last administration of 5-MeO-DMT or a pharmaceutically acceptable salt thereof; on day 1, for instance after about 24 hours; on day 7; on day 14; and/or on day 28 after the last administration of 5-MeO-DMT or a pharmaceutically acceptable salt thereof.

66. 5-MeO-DMT or a pharmaceutically acceptable salt thereof for use as in claim 64, wherein the improvement in the diagnosed disorder in a patient also suffering from associated negative thinking, as reflected by a reduction in the Clinical Global Impression-Severity (CGI-S) score, occurs not later than about 2 hours after the last administration of 5-MeO-DMT or a pharmaceutically acceptable salt thereof and wherein the improvement in the diagnosed disorder in a patient also suffering from associated negative thinking, as reflected by a reduction in the Clinical Global Impression-Severity (CGI-S) score, persists until at least 6 days after the last administration of 5-MeO-DMT or a pharmaceutically acceptable salt thereof; in particular until at least 14 days; more preferably until at least 28 days after the last administration of 5-MeO-DMT or a pharmaceutically acceptable salt thereof.

67. 5-MeO-DMT or a pharmaceutically acceptable salt thereof for use as in claims 1 to 5, wherein the patient is suffering from a sleep disturbance associated with the negative thinking.

68. 5-MeO-DMT or a pharmaceutically acceptable salt thereof for use as in claim 67, wherein the sleep disturbance is insomnia associated with the negative thinking.

69. 5-MeO-DMT or a pharmaceutically acceptable salt thereof for use as in claim 67 or 68, wherein the patient suffers from an idiopathic sleep disturbance associated with the negative thinking.

70. 5-MeO-DMT or a pharmaceutically acceptable salt thereof for use as in claims 67 to 69, wherein the patient suffers from a treatment resistant form of the disorder.

71. 5-MeO-DMT or a pharmaceutically acceptable salt thereof for use as in claim 67, 68 or 70, wherein the sleep disturbance occurs in a patient suffering from associated negative thinking and also from a mental or nervous system disorders, such as a disorder characterized by depressive episodes, for example, Major Depressive Disorder (MDD), Bipolar Disorder (BD), such as Bipolar I Disorder and Bipolar II Disorder, Postpartum Depression (PPD), Seasonal Affective Disorder and Persistent Depressive Disorder; Anxiety Disorder, for example Generalised Anxiety Disorder (GAD) and Social Anxiety Disorder (SAD); Obsessive Compulsive and Related Disorders, for example, Obsessive Compulsive Disorder (OCD) and Body Dysmorphic Disorder (BDD); Post-Traumatic Stress Disorder (PTSD); Pain Disorders, for example, Chronic Pain; Mental and Behavioural Disorder due to Psychoactive Substance Use, for example Substance Use Disorder (SUD); Psychotic Disorder, for example Schizophrenia; Dementia, for example Alzheimer's Dementia (AD); Eating Disorder; Attention Deficit Hyperactivity Disorder (ADHD); Personality Disorder, for example Schizotypal Personality Disorder and Borderline Personality Disorder (BPD) or from a medical health conditions leading to an associated mental or nervous system condition, such as Traumatic Brain Injury (TBI).

72. 5-MeO-DMT or a pharmaceutically acceptable salt thereof for use as in claim 71, wherein the treatment leads to an improvement in negative thinking and sleep disturbance and furthermore to an improvement in the associated mental or the nervous system disorder or the medical health condition leading to an associated mental or nervous system condition.

73. 5-MeO-DMT or a pharmaceutically acceptable salt thereof for use as in claims 67 to 72, wherein the treatment leads to an improvement in the sleep disturbance.

74. 5-MeO-DMT or a pharmaceutically acceptable salt thereof for use as in claim 73, wherein the improvement in the sleep disturbance, as reflected by a reduction in the Clinical Global Impression-Severity (CGI-S) score, is observed on day 1, for instance after about 24 hours; on day 7; on day 14; and/or on day 28 after the last administration of 5-MeO-DMT or a pharmaceutically acceptable salt thereof.

75. 5-MeO-DMT or a pharmaceutically acceptable salt thereof for use as in claim 73, wherein the improvement in the sleep disturbance, as reflected by a reduction in the Clinical Global Impression-Severity (CGI-S) score, occurs not later than about 24 hours after the last administration of 5-MeO-DMT or a pharmaceutically acceptable salt thereof and wherein the improvement in the sleep disturbance, as reflected by a reduction in the Clinical Global Impression-Severity (CGI-S) score, persists until at least 6 days after the last administration of 5-MeO-DMT or a pharmaceutically acceptable salt thereof; in particular until at least 14 days; more preferably until at least 28 days after the last administration of 5-MeO-DMT or a pharmaceutically acceptable salt thereof.

76. 5-MeO-DMT or a pharmaceutically acceptable salt thereof for use as in claim 73 wherein the improvement in the sleep disturbance, as reflected by a reduction in the Clinical Global Impression-Severity (CGI-S) score, is observed on day 7 after the last administration of 5-MeO-DMT or a pharmaceutically acceptable salt thereof and preferably persists until at least 14 days, more preferably until at least 28 days.

77. 5-MeO-DMT or a pharmaceutically acceptable salt thereof for use as in claims 1 to 76, wherein the 5-MeO-DMT or salt thereof is administered at a dose or in a dosage regimen that causes the patient to experience a peak psychedelic experience.

78. 5-MeO-DMT or a pharmaceutically acceptable salt thereof for use as in claims 1 to 77, wherein a dosage of about 4 mg to about 20 mg 5-MeO-DMT is administered, or wherein equimolar amounts of the pharmaceutically acceptable salt are administered instead of 5-MeO-DMT.

79. 5-MeO-DMT or a pharmaceutically acceptable salt thereof for use as in claims 1 to 77, wherein a dosage of about 6 mg; or of about 12 mg; or of about 18 mg is administered, or wherein equimolar amounts of the pharmaceutically acceptable salt are administered instead of 5-MeO-DMT.

80. 5-MeO-DMT or a pharmaceutically acceptable salt thereof for use as in claims 1 to 78, wherein the 5-MeO-DMT or salt thereof is administered in a first dosage amount for a first administration; and the 5-MeO-DMT or salt thereof is administered in zero to six subsequent administrations; wherein each subsequent administration uses a dosage amount higher than the previous administration unless the patient experiences a peak psychedelic experience.

81. 5-MeO-DMT or a pharmaceutically acceptable salt thereof for use as in claims 1 to 80, wherein the 5-MeO-DMT is administered in a dosage from about 2 mg to about 8 mg for a first administration, and then increased, unless the patient has already experienced a peak psychedelic experience, to a dosage from about 8 mg to about 14 mg for a second administration, and then increased, unless the patient has already experienced a peak psychedelic experience, to a dosage from about 14 mg to about 20 mg for a third administration, or wherein equimolar amounts of the pharmaceutically acceptable salt are administered instead of 5-MeO-DMT.

82. 5-MeO-DMT or a pharmaceutically acceptable salt thereof for use as in claim 81, wherein the first dosage of 5-MeO-DMT is about 6 mg, the second dosage of 5-MeO-DMT is about 12 mg, and the third dosage of 5-MeO-DMT is about 18 mg; or wherein equimolar amounts of the pharmaceutically acceptable salt are administered instead of 5-MeO-DMT.

83. 5-MeO-DMT or a pharmaceutically acceptable salt thereof for use as in claims 80 to 82, wherein the interval between two administrations is not less than 1 hour and not more than 24 hours, such as about 1 to 4 hours, preferably 1 to 2 hours.

84. 5-MeO-DMT or a pharmaceutically acceptable salt thereof for use as in claims 77 to 83, wherein the occurrence of a peak psychedelic experience is identified through achievement of at least 60% of the maximum possible score in each of the four subscales (mystical, positive mood, transcendence of time and space, and ineffability) of the 30-item revised Mystical Experience Questionnaire (MEQ30) or is identified through achievement of at least 60% of the maximum possible score of the Oceanic Boundlessness (OBN) dimension of the Altered States of Consciousness (ASC) questionnaire or is identified through achievement of a Peak Experience Scale (PES) Total Score of at least 75.

85. 5-MeO-DMT or a pharmaceutically acceptable salt thereof for use as in claim 84, wherein the occurrence of a peak psychedelic experience is identified through achievement of a Peak Experience Scale (PES) Total Score of at least 75.

86. 5-MeO-DMT or a pharmaceutically acceptable salt thereof for use as in claims 1 to 85, wherein the 5-MeO-DMT or a pharmaceutically acceptable salt thereof is administered via inhalation or by nasal, buccal or sublingual administration.

87. 5-MeO-DMT or a pharmaceutically acceptable salt thereof for use as in claim 86, wherein 5-MeO-DMT or a pharmaceutically acceptable salt thereof is administered in the form of an aerosol comprising (a) a pharmaceutically acceptable gas; (b) aerosol particles of 5-methoxy-N,N-dimethyltryptamine (5-MeO-DMT) or a pharmaceutically acceptable salt thereof, wherein the aerosol has an aerosol particle mass density of about 0.5 mg/l to about 18 mg/l, such as to about 12.5 mg/l.

88. 5-MeO-DMT or a pharmaceutically acceptable salt thereof for use as in claim 87, wherein the aerosol is generated by a) exposing a thin layer of 5-MeO-DMT or a pharmaceutically acceptable salt thereof, configured on a solid support, to thermal energy, and b) passing air over the thin layer to produce aerosol particles.

89. 5-MeO-DMT or a pharmaceutically acceptable salt thereof for use as in claims 86 to 88, wherein the dosage amount of 5-MeO-DMT or a pharmaceutically acceptable salt to be administered to the patient is inhaled with a single breath.

90. 5-MeO-DMT for use as in claims 86 to 89, wherein the 5-MeO-DMT is used in the form of the free base.
